SEQUENCE LISTING (78 sequences)

SEQ ID NO: 1 - protein, 203 aa, Artificial Sequence
  synthesized, a fusion protein comprising: a fragment of TRAIL protein, a fragment of human fetoprotein and a cleavage site recognized by urokinase uPA.
LSEDKLLACGEGAADIIIGHLCIRHEMTPVNPGRVVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 2 - protein, 178 aa, Artificial Sequence
  synthesized, a fusion protein comprising: a fragment of TRAIL protein, a fragment of human fetoprotein and a cleavage site recognized by urokinase uPA.
EMTPVNPGRVVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 179 aa, Artificial Sequence
  synthesized, a fusion protein comprising: a fragment of TRAIL protein, a fragment of human fetoprotein and cleavage sites recognized by uPA and MMP.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGPLGLAGRVVREMTPVNPG

SEQ ID NO: 4 - protein, 204 aa, Artificial Sequence
  synthesized, a fusion protein comprising: a fragment of TRAIL protein, a fragment of human fetoprotein and cleavage sites recognized by uPA and MMP.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGPLGLAGRVVRLSEDKLLACGEGAADIIIGHLCIRHEMTPVNPG

SEQ ID NO: 5 - protein, 230 aa, Artificial Sequence
  synthesized, a fusion protein comprising: a fragment of TRAIL protein, a fragment of human fetoprotein and cleavage sites recognized by uPA and MMP.
LSEDKLLACGEGAADIIIGHLCIRHEMTPVNPGRVVRPLGLAGTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 6 - protein, 238 aa, Artificial Sequence
  synthesized, a fusion protein comprising: a fragment of TRAIL protein, a fragment of human fetoprotein, cleavage sites recognized by uPA and MMP and a flexible cysteine - alanine linker.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGCAACAAACPLGLAGRVVRLSEDKLLACGEGAADIIIGHLCIRHEMTPVNPG

SEQ ID NO: 7 - protein, 213 aa, Artificial Sequence
  synthesized, a fusion protein comprising: a fragment of TRAIL protein, a fragment of human fetoprotein, cleavage sites recognized by uPA and MMP and a flexible cysteine - alanine linker.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGCAACAAACPLGLAGRVVREMTPVNPG

SEQ ID NO: 8 - protein, 197 aa, Artificial Sequence
  synthesized, a fusion protein comprising: a fragment of TRAIL protein, a peptide derived from p21WAF protein, cleavage sites recognized by uPA and MMP and an antennapedia transporting domain.
KRRQTSATDFYHSKRRLIFSKPRRPYRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - protein, 231 aa, Artificial Sequence [sequence omitted]

SEQ ID NO: 10 - protein, 200 aa, Artificial Sequence
  synthesized, a fusion protein comprising: a fragment of TRAIL protein, a peptide analogue of a domain binding to FGF-2 receptor, cleavage sites recognized by uPA and MMP and cysteine-alanine and glycine steric linkers.
MWYRPDLDERKQQKRERVVRPLGLAGGGCAAACAACGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - protein, 233 aa, Artificial Sequence [sequence omitted]

SEQ ID NO: 12 - protein, 590 aa, Artificial Sequence
  synthesized, a fusion protein comprising: a fragment of TRAIL protein, an arginine deiminase from Mycoplasma arginini, cleavage sites recognized by urokinase uPA and metalloprotease MMP and glycine-serine steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGSGPLGLAGRVVRGGGSGMSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW

SEQ ID NO: 13 - protein, 187 aa, Artificial Sequence
  synthesized, a fusion protein comprising: a fragment of TRAIL protein, a peptide from p16 protein, cleavage sites recognized by urokinase uPA and metalloprotease MMP and an antennapedia transporting domain.
FLDTLVVLHRKPRRPYRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 14 - protein, 203 aa, Artificial Sequence
  synthesized, a fusion protein comprising: a fragment of TRAIL protein, a fragment of MEK-1 protein - an inhibitor of ERK activation, cleavage sites recognized by  uPA and MMP, an antennapedia transporting domain and a glycine- cysteine linker.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGSPLGLAGRVVRDRQIKIWFQNRRMKWKKGMPKKKPTPIQLN

SEQ ID NO: 15 - protein, 205 aa, Artificial Sequence [sequence omitted]

SEQ ID NO: 16 - protein, 183 aa, Artificial Sequence
  synthesized, a fusion protein comprising: a fragment of TRAIL protein, a peptide inhibitor of E2F, cleavage sites recognized by urokinase uPA and metalloprotease MMP and an antennapedia transporting domain.
FWLRFTKPRRPYRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 17 - protein, 190 aa, Artificial Sequence
  synthesized, a fusion protein comprising: a fragment of TRAIL protein, a fragment of tubulin peptide, cleavage sites recognized by urokinase uPA and metalloprotease MMP and an arginine transporting domain.
FRRKAFLHWYTGRRRRRRRRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 18 - protein, 187 aa, Artificial Sequence
  synthesized, a fusion protein comprising: a fragment of TRAIL protein, a fragment of tubulin peptide, cleavage sites recognized by urokinase uPA and metalloprotease MMP and an arginine transporting domain.
WCPTGFKVGRRRRRRRRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 19 - protein, 196 aa, Artificial Sequence
  synthesized, a fusion protein comprising: a fragment of TRAIL protein, a melittin peptide and cleavage sites recognized by urokinase uPA and metalloprotease MMP.
GIGARLKVLTTGLPRISWIKRKRQQRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 20 - protein, 184 aa, Artificial Sequence
  synthesized, a fusion protein comprising: a fragment of TRAIL protein, a peptide C2 derived from bee defensin, cleavage sites recognized by urokinase uPA and metalloprotease MMP and an arginine transporting domain.
ALYLAIRRRRRRRRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 21 - protein, 189 aa, Artificial Sequence
  synthesized, a fusion protein comprising: a fragment of TRAIL protein, a peptide binding to FGF-2 ligand, cleavage sites recognized by urokinase uPA and metalloprotease MMP and a steric glycine linker.
APSGHYKGRVVRPLGLAGAPSGHYKGGGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 22 - protein, 188 aa, Artificial Sequence
  synthesized, a fusion protein comprising: a fragment of TRAIL protein, a lasioglossin LL2 peptide and cleavage sites recognized by urokinase uPA and metalloprotease MMP.
VNWKKVLGKIIKVAKRVVRPLGLAGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 23 - protein, 193 aa, Artificial Sequence
  synthesized, a fusion protein comprising: a fragment of TRAIL protein, a peptide acting as an inhibitor of interactions RasGAP - Aurora B, cleavage sites recognized by uPA and MMP, an arginine transporting domain and a cysteine stabilizing residue.
KWVVSHARLMYSFRRRRRRRRRVVRPLGLAGCRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 24 - protein, 243 aa, Artificial Sequence
  synthesized, a fusion protein comprising: a fragment of TRAIL protein, a fragment of p16 peptide fused with a 17-amino-acid transporting domain of antennapedia, cleavage sites recognized by uPA and MMP and cysteine-alanine linker.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGCAACAAACPLGLAGRVVRDAAREGFLDTLVVLHRAGARVVRQIKIWFQNRRMKWKK

SEQ ID NO: 25 - protein, 199 aa, Artificial Sequence [sequence omitted]

SEQ ID NO: 26 - protein, 16 aa, Artificial Sequence
  synthesized, an analogue of FGF-R binding peptide
  ref <301>: AUTHORS: Kono K, Ueba T, Takahashi JA, Murai N, Hashimoto N, Myoumoto A, Itoh N,Fukumoto M
  ref <302>: TITLE: In vitro growth suppression of human glioma cells by a 16-mer oligopeptide: a potential new treatment modality for malignant glioma.
  ref <303>: JOURNAL: . J Neurooncol
  ref <304>: VOLUME: 63
  ref <305>: ISSUE: 2
  ref <306>: PAGES: 163-71
  ref <307>: DATE: 2003-07-30
MWYRPDLDERKQQKRE

SEQ ID NO: 27 - protein, 34 aa, Homo sapiens
  ref <308>: DATABASE ACCESSION NUMBER: GenBank/AAB58754.1
  ref <309>: DATABASE ENTRY DATE: 2008-10-16
  ref <313>: RELEVANT RESIDUES IN SEQ ID NO: (464)..(496)
LSEDKLLACGEGAADIIIGHLCIRHEMTPVNPGR

SEQ ID NO: 28 - protein, 8 aa, Homo sapiens
  ref <308>: DATABASE ACCESSION NUMBER: GenBank/ AAB58754.1
  ref <309>: DATABASE ENTRY DATE: 2008-10-16
  ref <313>: RELEVANT RESIDUES IN SEQ ID NO: (489)..(496)
EMTPVNPG

SEQ ID NO: 29 - protein, 20 aa, Homo sapiens
  ref <308>: DATABASE ACCESSION NUMBER: GenBank/ BAG70236.1
  ref <309>: DATABASE ENTRY DATE: 2008-08-02
  ref <313>: RELEVANT RESIDUES IN SEQ ID NO: (141)..(160)
KRRQTSATDFYHSKRRLIFS

SEQ ID NO: 30 - protein, 18 aa, Homo sapiens
  ref <308>: DATABASE ACCESSION NUMBER: NCBI/
  ref <309>: DATABASE ENTRY DATE: 2010-03-04
  ref <313>: RELEVANT RESIDUES IN SEQ ID NO: (417)..(431)
GGGFQLRQPPLVPSRKGE

SEQ ID NO: 31 - protein, 410 aa, Mycoplasma arginini
  ref <308>: DATABASE ACCESSION NUMBER: GenBank/ CAA38080.1
  ref <309>: DATABASE ENTRY DATE: 2004-09-09
  ref <313>: RELEVANT RESIDUES IN SEQ ID NO: (1)..(410)
MSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW

SEQ ID NO: 32 - protein, 10 aa, Homo sapiens
  ref <308>: DATABASE ACCESSION NUMBER: GenBank/ ABS32187.1
  ref <309>: DATABASE ENTRY DATE: 2007-07-25
  ref <313>: RELEVANT RESIDUES IN SEQ ID NO: (40)..(49)
FLDTLVVLHR

SEQ ID NO: 33 - protein, 38 aa, Homo sapiens
  ref <308>: DATABASE ACCESSION NUMBER: NCBI NP_001182061.1
  ref <309>: DATABASE ENTRY DATE: 2011-02-19
  ref <313>: RELEVANT RESIDUES IN SEQ ID NO: (84)..(103)
DAAREGFLDTLVVLHRAGARVVRQIKIWFQNRRMKWKK

SEQ ID NO: 34 - protein, 13 aa, Homo sapiens
  ref <308>: DATABASE ACCESSION NUMBER: NCBI NP_002746.1
  ref <309>: DATABASE ENTRY DATE: 2010-04-19
  ref <313>: RELEVANT RESIDUES IN SEQ ID NO: (1)..(12)
GMPKKKPTPIQLN

SEQ ID NO: 35 - protein, 15 aa, Homo sapiens
  ref <308>: DATABASE ACCESSION NUMBER: GenBank/ AAH03574.1
  ref <309>: DATABASE ENTRY DATE: 2006-07-15
  ref <313>: RELEVANT RESIDUES IN SEQ ID NO: (10)..(24)
AVTDHPDRLWAWEKF

SEQ ID NO: 36 - protein, 6 aa, Homo sapiens
  ref <302>: TITLE: Transcription factor E2F DNA-binding domain inhibitor peptides and their use
  ref <308>: DATABASE ACCESSION NUMBER: USPTO
  ref <309>: DATABASE ENTRY DATE: 2003-01-16
  ref <310>: PATENT DOCUMENT NUMBER: US6841385
  ref <311>: PATENT FILING DATE: 2001-07-26
  ref <312>: PUBLICATION DATE: 2003-01-16
FWLRFT

SEQ ID NO: 37 - protein, 13 aa, Homo sapiens
  ref <301>: AUTHORS: Pieraccini, S., Salamino, G., Cappelletti, G., Cartelli, D., Francescato, P., Speranza, G., Manisto, P., Sironi, M
  ref <302>: TITLE: In silico design of tubulin - targeted antimitotic peptides
  ref <303>: JOURNAL: Nature Chemistry
  ref <304>: VOLUME: 23
  ref <306>: PAGES: 642 - 648
  ref <307>: DATE: 2009-09-23
FRRKAFLHWYTGR

SEQ ID NO: 38 - protein, 10 aa, Homo sapiens
  ref <301>: AUTHORS: Pieraccini, S., Salamino, G., Cappelletti, G., Cartelli, D., Francescato, P., Speranza, G., Manisto, P., Sironi, M.,
  ref <302>: TITLE: In silico design of tubulin - targeted antimitotic peptides,
  ref <303>: JOURNAL: Nature Chemistry
  ref <304>: VOLUME: 1
  ref <306>: PAGES: 642 - 648
  ref <307>: DATE: 2009-10-23
WCPTGFKVGR

SEQ ID NO: 39 - protein, 25 aa, Apis mellifera
  ref <308>: DATABASE ACCESSION NUMBER: GenBank/ AAU87881.1
  ref <309>: DATABASE ENTRY DATE: 2004-10-04
  ref <313>: RELEVANT RESIDUES IN SEQ ID NO: (1)..(25)
GIGARLKVLTTGLPRISWIKRKRQQ

SEQ ID NO: 40 - protein, 6 aa, Artificial Sequence
  synthetic, a 6-amino acid peptide C2 derived from bee defensin
  ref <301>: AUTHORS: Iwasaki T, Ishibashi J, Kubo M, Taylor D, Yamakawa M.
  ref <302>: TITLE: Multiple functions of short synthetic enantiomeric peptides based on beetle defensins
  ref <303>: JOURNAL: Biosci Biotechnol Biochem
  ref <304>: VOLUME: 73
  ref <305>: ISSUE: 3
  ref <306>: PAGES: 683-687
  ref <307>: DATE: 2009-03-23
ALYLAI

SEQ ID NO: 41 - protein, 8 aa, Homo sapiens
  ref <301>: AUTHORS: Kono K, Ueba T, Takahashi JA, Murai N, Hashimoto N, Myoumoto A, Itoh N,Fukumoto M
  ref <302>: TITLE: In vitro growth suppression of human glioma cells by a 16-mer oligopeptide: a potential new treatment modality for malignant glioma.
  ref <303>: JOURNAL: J Neurooncol
  ref <304>: VOLUME: 63
  ref <305>: ISSUE: 2
  ref <306>: PAGES: 163-171
  ref <307>: DATE: 2003-07-30
APSGHYKG

SEQ ID NO: 42 - protein, 15 aa, Lasioglossum laticeps
  ref <301>: AUTHORS: Cerovsky V, Budesinsky M, Hovorka O, Cvacka J, Voburka Z, Slaninova J, Borovickova L, Fucik V, Bednarova L, Votruba I, Straka
  ref <302>: TITLE: Lasioglossins/ three novel antimicrobial peptides from the venom of the eusocial bee Lasioglossum laticeps (Hymenoptera/ Halictidae).
  ref <303>: JOURNAL: JChembiochem.
  ref <304>: VOLUME: 10
  ref <305>: ISSUE: 12
  ref <306>: PAGES: 2089-2099
  ref <307>: DATE: 2009-08-17
VNWKKVLGKIIKVAK

SEQ ID NO: 43 - protein, 13 aa, Artificial Sequence
  synthetic, a 13-amino acid peptide acting as an inhibitor of interactions RasGAP - Aurora B
  ref <301>: AUTHORS: Pamonsinlapatham P, Hadj-Slimane R, Raynaud F, Bickle M, Corneloup C, Barthelaix A, Lepelletier Y, Mercier P, Schapira M, Samson J, Mathieu AL, Hugo N, Moncorge O, Mikaelian I, Dufour S, Garbay C, Colas P.
  ref <302>: TITLE: A RasGAP SH3 peptide aptamer inhibits RasGAP-Aurora interaction and induces caspase-independent tumor cell death
  ref <303>: JOURNAL: PLoS One
  ref <304>: VOLUME: 3
  ref <305>: ISSUE: 8)/e2902
  ref <306>: PAGES: doi:10.1371/journal.pone.0002902
  ref <307>: DATE: 2008-08-06
KWVVSHARLMYSF

SEQ ID NO: 44 - protein, 27 aa, Streptococcus pneumoniae
  ref <301>: AUTHORS: Dong Gun Lee1 , Kyung-Soo Hahm2 , Yoonkyung Park2 , Hai-Young Kim3 , Weontae Lee3 , Sung-Chul Lim4 , Youn-Kyung Seo5 and Cheol-Hee Choi5
  ref <302>: TITLE: Functional and structural characteristics of anticancer peptide Pep27 analogues
  ref <303>: JOURNAL: Cancer Cell International
  ref <304>: VOLUME: 5
  ref <305>: ISSUE: 21
  ref <306>: PAGES: doi:10.1186/1475-2867-5-21
  ref <307>: DATE: 2005-07-11
MWKWFHNVLSWWWLLADKRPARDYNRK

SEQ ID NO: 45 - protein, 6 aa, Artificial Sequence
  synthesized, a protease cutting sequence
PLGLAG

SEQ ID NO: 46 - protein, 4 aa, Artificial Sequence
  synthesized, a protease cutting sequence
RVVR

SEQ ID NO: 47 - protein, 8 aa, Artificial Sequence
  synthesized, an artificial sequence of a steric linker
CAACAAAC

SEQ ID NO: 48 - protein, 17 aa, Drosophila melanogaster
  ref <301>: AUTHORS: Derossi D, Calvet S, Trembleau A, Brunissen A, Chassaing G, Prochiantz A
  ref <302>: TITLE: Cell
  ref <303>: JOURNAL: J Biol Chem
  ref <304>: VOLUME: 271
  ref <305>: ISSUE: 30
  ref <306>: PAGES: 18188-93
  ref <307>: DATE: 1996-07-26
DRQIKIWFQNRRMKWKK

SEQ ID NO: 49 - protein, 6 aa, Drosophila melanogaster
  ref <301>: AUTHORS: Derossi D, Calvet S, Trembleau A, Brunissen A, Chassaing G, Prochiantz A.
  ref <302>: TITLE: Cell internalization of the third helix of the Antennapedia homeodomain is
  ref <303>: JOURNAL: J Biol Chem
  ref <304>: VOLUME: 271
  ref <305>: ISSUE: 30
  ref <306>: PAGES: 18188-93
  ref <307>: DATE: 1996-07-26
KPRRPY

SEQ ID NO: 50 - DNA, 609 bp, Artificial Sequence
  synthesized, encoding a fusion protein comprising: a fragment of TRAIL protein, a fragment of human fetoprotein and a cleavage site recognized by urokinase uPA.
ctgtctgaagacaaactgctggcatgcggcgaaggcgcggcagacattatcatcggccacctgtgcattcgtcatgaaatgacgccggttaacccgggtcgtgttgttcgtgaacgtggtccgcaacgcgttgcggcacatattacgggtacccgtggccgcagcaacacgctgagctctccgaattcgaaaaatgaaaaagcactgggccgcaaaattaactcgtgggaaagcagtcgttctggtcacagctttctgtcgaatctgcacctgcgcaatggtgaactggtgattcatgaaaaaggcttttactatatctattctcagacgtattttcgttttcaggaagaaattaaagaaaacaccaaaaatgacaaacagatggtgcagtacatttacaaatacaccagttacccggacccgattctgctgatgaaaagcgcccgtaactcatgctggagcaaagacgctgaatatggcctgtattctatttatcagggtggcatcttcgaactgaaagaaaacgatcgtatttttgtttcggtgaccaacgaacacctgattgatatggatcatgaagcatcgtttttcggcgcgtttctggtcggc

SEQ ID NO: 51 - DNA, 534 bp, Artificial Sequence
  synthesized, encoding a fusion protein comprising: a fragment of TRAIL protein, a fragment of human fetoprotein and a cleavage site recognized by urokinase uPA.
gaaatgacgccggtgaatccgggtcgtgttgttcgtgaacgtggtccgcaacgcgttgcggcacatattacgggtacccgtggccgcagcaacacgctgagctctccgaattcgaaaaatgaaaaagcactgggccgcaaaattaactcgtgggaaagcagtcgttctggtcacagctttctgtcgaatctgcacctgcgcaatggtgaactggtgattcatgaaaaaggcttttactatatctattctcagacgtattttcgttttcaggaagaaattaaagaaaacaccaaaaatgacaaacagatggtgcagtacatttacaaatacaccagttacccggacccgattctgctgatgaaaagcgcccgtaactcatgctggagcaaagacgctgaatatggcctgtattctatttatcagggtggcatcttcgaactgaaagaaaacgatcgtatttttgtttcggtgaccaacgaacacctgattgatatggatcatgaagcatcgtttttcggcgcgtttctggtcggc

SEQ ID NO: 52 - DNA, 537 bp, Artificial Sequence
  synthesized, encoding a fusion protein comprising: a fragment of TRAIL protein, a fragment of human fetoprotein and cleavage sites recognized by uPA and MMP.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtccgctgggtctggcaggtcgtgttgttcgtgaaatgacaccggttaatccgggt

SEQ ID NO: 53 - DNA, 612 bp, Artificial Sequence
  synthesized, encoding a fusion protein comprising: a fragment of TRAIL protein, a fragment of human fetoprotein and cleavage sites recognized by uPA and MMP.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtccgctgggtctggcaggtcgtgttgttcgtctgagcgaagataaactgctggcatgtggtgaaggtgcagccgatattattattggccatctgtgcattcgtcatgaaatgacaccggttaatccgggt

SEQ ID NO: 54 - DNA, 690 bp, Artificial Sequence
  synthesized, encoding a fusion protein comprising: a fragment of TRAIL protein, a fragment of human fetoprotein and cleavage sites recognized by uPA and MMP.
ctgagcgaagataaactgctggcatgtggtgaaggtgcagcagatattattattggtcatctgtgcattcgccatgaaatgacaccggttaatccgggtcgtgttgttcgtcctctgggtctggcaggcaccagcgaagaaaccattagcaccgttcaggaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 55 - DNA, 714 bp, Artificial Sequence
  synthesized, encoding a fusion protein comprising: a fragment of TRAIL protein, a fragment of human fetoprotein, cleavage sites recognized by uPA and MMP and a flexible cysteine - alanine linker.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggttgtgcagcatgtgcagccgcatgtccgctgggtctggcaggtcgtgttgttcgtctgagcgaagataaactgctggcatgtggtgaaggtgcagcagatattatcattggtcatctgtgcattcgccatgaaatgacaccggttaatccgggt

SEQ ID NO: 56 - DNA, 639 bp, Artificial Sequence
  synthesized, encoding a fusion protein comprising: a fragment of TRAIL protein, a fragment of human fetoprotein, cleavage sites recognized by uPA and MMP and a flexible cysteine - alanine linker.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggttgtgcagcatgtgcagccgcatgtccgctgggtctggcaggtcgtgttgttcgtgaaatgacaccggttaatccgggt

SEQ ID NO: 57 - DNA, 591 bp, Artificial Sequence
  synthesized, encoding a fusion protein comprising: a fragment of TRAIL protein, a peptide derived from p21WAF protein, cleavage sites recognized by uPA and MMP and an antennapedia transporting domain.
aaacgtcgtcagaccagcgcaaccgatttttatcatagcaaacgtcgcctgatttttagcaaaccgcgtcgtccgtatcgtgttgttcgtccgctgggtctggcaggtcgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatttataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 58 - DNA, 693 bp, Artificial Sequence
  synthesized, encoding a fusion protein comprising: a fragment of TRAIL protein, a peptide derived from p21WAF protein, cleavage sites recognized by uPA and MMP, a cysteine-alanine steric linker and an antennapedia transporting domain.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatataccagctatccggatccgatcctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggttgtgcagcatgtgcagccgcatgtccgctgggtctggcaggtcgtgttgttcgtaaacgtcgtcagaccagcgcaaccgatttttatcatagcaaacgtcgcctgatctttagcaaaccgcgtcgtccgtat

SEQ ID NO: 59 - DNA, 600 bp, Artificial Sequence
  synthesized, encoding a fusion protein comprising: a fragment of TRAIL protein, a peptide analogue of a domain binding to FGF-2 receptor, cleavage sites recognized by uPA and MMP and cysteine-alanine and glycine steric linkers.
atgtggtaccgtccggacctggacgaacgtaaacagcagaaacgtgaacgtgttgttcgtccgctgggtctggcgggtggtggttgcgcggcggcgtgcgcggcgtgcggtggtcagcgtgttgcggcgcacatcaccggtacccgtggtcgttctaacaccctgtcttctccgaactctaaaaacgaaaaagcgctgggtcgtaaaatcaactcttgggaatcttctcgttctggtcactctttcctgtctaacctgcacctgcgtaacggtgaactggttatccacgaaaaaggtttctactacatctactctcagacctacttccgtttccaggaagaaatcaaagaaaacaccaaaaacgacaaacagatggttcagtacatctacaaatacacctcttacccggacccgatcctgctgatgaaatctgcgcgtaactcttgctggtctaaagacgcggaatacggtctgtactctatctaccagggtggtatcttcgaactgaaagaaaacgaccgtatcttcgtttctgttaccaacgaacacctgatcgacatggaccacgaagcgtctttcttcggtgcgttcctggttggt

SEQ ID NO: 60 - DNA, 699 bp, Artificial Sequence
  synthesized, encoding a fusion protein comprising: a fragment of TRAIL protein, a fragment of peptide DD2 derived from DOC-2/DAB2, cleavage sites recognized by uPA and MMP, an arginine transporting domain and a cysteine-alanine- glycine steric linker.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggttgtgcagcatgtgcagccgcatgtggtggtggtccgctgggtctggcaggtcgtgttgttcgtcgtcgtcgccgtcgtcggcgtggtggtggttttcagctgcgtcagcctccgctggttccgagccgtaaaggtgaa

SEQ ID NO: 61 - DNA, 1770 bp, Artificial Sequence
  synthesized, encoding a fusion protein comprising: a fragment of TRAIL protein, an arginine deiminase from Mycoplasma arginini, cleavage sites recognized by urokinase uPA and metalloprotease MMP and glycine-serine steric linkers.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtagcggtccgctgggtctggcaggtcgtgttgttcgtggtggtggttcaggtatgagcgtttttgatagcaaattcaaaggcatccacgtgtatagcgaaattggtgagctggaaagcgttctggttcatgaaccgggtcgtgaaattgattatattacaccggcacgtctggatgaactgctgtttagcgcaattctggaaagccatgatgcacgtaaagaacataaacagtttgtggcagaactgaaagccaacgatattaatgtggtggaactgattgatctggttgcagaaacctatgatctggcaagccaagaagcaaaagataaactgatcgaagaatttctggaagatagcgaaccggttctgagcgaagaacataaagttgttgtgcgcaattttctgaaagccaaaaaaaccagccgtgaactggtggaaattatgatggcaggcatcaccaaatatgatctgggtattgaagccgatcatgaactgattgttgatccgatgccgaatctgtattttacccgtgatccgtttgcaagcgttggtaatggtgttaccattcattacatgcgttataaagtgcgtcagcgtgaaaccctgtttagccgttttgtttttagcaatcacccgaaactgattaataccccgtggtattatgatccgagcctgaaactgagcattgaaggtggtgatgtgttcatctataacaatgataccctggttgttggtgttagcgaacgtaccgatctgcagaccgttaccctgctggcaaaaaatattgtggccaataaagaatgcgaatttaaacgcattgtggccattaatgttccgaaatggaccaatctgatgcatctggatacctggctgaccatgctggataaagacaaatttctgtatagcccgattgccaacgacgtgtttaaattttgggattatgacctggttaatggtggtgcagaaccgcagccggttgaaaatggtctgccgctggaaggtctgctgcagagcattattaataaaaaaccggtgctgattccgattgccggtgaaggtgcaagccagatggaaattgaacgtgaaacccattttgatggcaccaattatctggcaattcgtccgggtgttgttattggttatagccgtaatgagaaaaccaatgcagcactggaagcagcaggtattaaagttctgccgtttcatggtaatcagctgagcctgggtatgggtaatgcacgttgtatgagcatgccgctgagccgtaaagatgttaaatgg

SEQ ID NO: 62 - DNA, 561 bp, Artificial Sequence
  synthesized, encoding a fusion protein comprising: a fragment of TRAIL protein, a peptide from p16 protein, cleavage sites recognized by urokinase uPA and metalloprotease MMP and an antennapedia transporting domain.
tttctggataccctggttgttctgcatcgtaaaccgcgtcgtccgtatcgtgttgttcgtccgctgggtctggcaggtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 63 - DNA, 609 bp, Artificial Sequence
  synthesized, encoding a fusion protein comprising: a fragment of TRAIL protein, a fragment of MEK-1 protein - an inhibitor of ERK activation, cleavage sites recognized by  uPA and MMP, an antennapedia transporting domain and a glycine-cysteine linker.
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtagtccgctgggtctggcaggtcgtgttgttcgtgatcgtcagattaaaatttggtttcagaatcgtcgcatgaaatggaaaaaaggcatgccgaaaaaaaaaccgaccccgattcagctgaat

SEQ ID NO: 64 - DNA, 615 bp, Artificial Sequence
  synthesized, encoding a fusion protein comprising: a fragment of TRAIL protein, a fragment of PH domain of TCL1 protein - acting as Akt coactivator, cleavage sites recognized by uPA and MMP, an antennapedia transporting domain and a glycine-cysteine linker.
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtagtccgctgggtctggcaggtcgtgttgttcgtgatcgtcagattaaaatttggtttcagaatcgtcgcatgaaatggaaaaaagcagttaccgatcatccggatcgtctgtgggcatgggaaaaattt

SEQ ID NO: 65 - DNA, 549 bp, Artificial Sequence
  synthesized, encoding a fusion protein comprising: a fragment of TRAIL protein, a peptide inhibitor of E2F, cleavage sites recognized by urokinase uPA and metalloprotease MMP and an antennapedia transporting domain.
ttttggctgcgttttaccaaaccgcgtcgtccgtatcgtgttgttcgtccgctgggtctggcaggtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 66 - DNA, 570 bp, Artificial Sequence
  synthesized, encoding a fusion protein comprising: a fragment of TRAIL protein, a fragment of tubulin peptide, cleavage sites recognized by urokinase uPA and metalloprotease MMP and an arginine transporting domain.
tttcgtcgtaaagcatttctgcattggtataccggtcgtcgtcgccgtcgccgtcgtcgtgttgttcgtcctctgggtctggcaggtcgtgttgcagcacatattaccggcacccgtggtcgtagcaataccctgagcagcccgaatagcaaaaatgagaaagcactgggtcgcaaaatcaatagctgggaaagcagccgtagcggtcatagctttctgagcaatctgcatctgcgtaatggtgaactggtgattcacgagaaaggcttctattacatctatagccagacctatttccgcttccaagaggaaattaaagagaacaccaaaaacgacaaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 67 - DNA, 561 bp, Artificial Sequence
  synthesized, encoding a fusion protein comprising: a fragment of TRAIL protein, a fragment of tubulin peptide, cleavage sites recognized by urokinase uPA and metalloprotease MMP and an arginine transporting domain.
tggtgtccgaccggttttaaagttggacgccgtcgtcgtcgccgtcgtcgtgttgttcgtcctctgggtctggcaggtcgtgttgcagcacatattaccggcacccgtggtcgtagcaataccctgagcagcccgaatagcaaaaatgagaaagcactgggtcgcaaaattaacagctgggaaagcagccgtagcggtcatagctttctgagcaatctgcatctgcgtaatggtgaactggtgattcatgagaaaggcttctattatatctatagccagacctattttcgctttcaagaggaaattaaagagaataccaaaaacgataaacaaatggtgcagtacatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 68 - DNA, 588 bp, Artificial Sequence
  synthesized, encoding a fusion protein comprising: a fragment of TRAIL protein, a melittin peptide and cleavage sites recognized by urokinase uPA and metalloprotease MMP.
ggcattggtgcacgtctgaaagttctgaccaccggtctgcctcgtattagctggattaaacgtaaacgtcagcagcgtgttgttcgtccgctgggtctggcaggtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 69 - DNA, 552 bp, Artificial Sequence
  synthesized, encoding a fusion protein comprising: a fragment of TRAIL protein, a peptide C2 derived from bee defensin, cleavage sites recognized by urokinase uPA and metalloprotease MMP and an arginine transporting domain.
gcactgtatctggcaattcgtcgtcgtcgtcgccgtcgtcgtgttgttcgtccgctgggtctggcaggtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagagaataccaaaaacgataagcagatggtgcagtacatttataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 70 - DNA, 567 bp, Artificial Sequence
  synthesized, encoding a fusion protein comprising: a fragment of TRAIL protein, a peptide binding to FGF-2 ligand, cleavage sites recognized by urokinase uPA and metalloprotease MMP and a steric glycine linker.
gcccccagtggacattataagggaagggtcgtcaggcccctaggactagccggagcccccagtggacattataagggaggaggaagggtcgccgcccatataacaggaacaaggggaaggagtaatacactaagtagtcccaatagtaagaatgagaaggccctaggaaggaagataaatagttgggagagtagtaggagtggacatagttttctaagtaatctacatctaaggaatggagagctagtcatacatgagaagggattttattatatatatagtcaaacatattttaggtttcaagaggagataaaggagaatacaaagaatgataagcaaatggtccaatatatatataagtatacaagttatcccgatcccatactactaatgaagagtgccaggaatagttgttggagtaaggatgccgagtatggactatatagtatatatcaaggaggaatatttgagctaaaggagaatgataggatatttgtcagtgtcacaaatgagcatctaatagatatggatcatgaggccagtttttttggagcctttctagtcgga

SEQ ID NO: 71 - DNA, 564 bp, Artificial Sequence
  synthesized, encoding a fusion protein comprising: a fragment of TRAIL protein, a lasioglossin LL2 peptide and cleavage sites recognized by urokinase uPA and metalloprotease MMP.
gtgaattggaaaaaagtgctgggcaaaattattaaagtggccaaacgtgttgttcgtccgctgggtctggcaggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgacaaacaaatggtgcaatatatctacaaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 72 - DNA, 579 bp, Artificial Sequence
  synthesized, a fusion protein comprising: a fragment of TRAIL protein, a peptide acting as an inhibitor of interactions RasGAP - Aurora B, cleavage sites recognized by uPA and MMP, an arginine transporting domain and a cysteine stabilizing residue.
aaatgggttgttagccatgcacgtctgatgtatagctttcgtcgtcgtcgccgtcggcgtcgtcgtgttgttcgtccgctgggtctggcaggttgtcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgacaaacaaatggtgcaatatatctacaaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 73 - DNA, 729 bp, Artificial Sequence
  synthesized, encoding a fusion protein comprising: a fragment of TRAIL protein, a fragment of p16 peptide fused with a 17-amino-acid transporting domain of antennapedia, cleavage sites recognized by uPA and MMP and cysteine-alanine linker.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggttgtgcagcatgtgcagccgcatgtccgctgggtctggcaggtcgtgttgttcgtgatgcagcacgtgaaggttttctggataccctggttgttctgcatcgtgccggtgcacgtgttgtgcgtcagattaaaatctggtttcagaatcgtcgcatgaaatggaaaaag

SEQ ID NO: 74 - DNA, 597 bp, Artificial Sequence
  synthesized, encoding a fusion protein comprising: a fragment of TRAIL protein, an analogue of Pep27 peptide and cleavage sites recognized by urokinaze uPA and metalloprotease MMP.
atgtggaaatggtttcataatgttctgagctggtggtggctgctggcagataaacgtccggcacgtgattataatcgtaaacgtgttgttcgtccgctgggtctggcaggtcagcgtgttgcagcacatattaccggtacccgtggtcgtagcaataccctgagcagcccgaatagcaaaaatgaaaaagcactgggtcgtaaaattaatagctgggaaagcagccgtagcggtcatagctttctgagcaatctgcatctgcgtaatggtgaactggttattcatgaaaaaggtttttattatatttatagccagacctattttcgttttcaggaagaaattaaagaaaataccaaaaatgataaacagatggttcagtatatttataaatataccagctatccggatccgattctgctgatgaaaagcgcacgtaatagctgttggagcaaagatgcagaatatggtctgtatagcatttatcagggtggtatttttgaactgaaagaaaatgatcgtatttttgttagcgttaccaatgaacatctgattgatatggatcatgaagcaagcttttttggtgcatttctggttggt

SEQ ID NO: 75 - protein, 212 aa, Artificial Sequence
  synthesized, a fusion protein comprising: a fragment of TRAIL protein, a peptide derived from p21WAF protein, an antennapedia transporting domain, cleavage sites recognized by urokinase uPA and metalloprotease MMP and a flexible linker.
KRRQTSATDFYHSKRRLIFSKPRRPYRVVRPLGLAGGGGCAAACAACGSGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 76 - DNA, 636 bp, Artificial Sequence
  synthesized, encoding a fusion protein comprising: a fragment of TRAIL protein, a peptide derived from p21WAF protein, an antennapedia transporting domain, cleavage sites recognized by urokinase uPA and metalloprotease MMP and a flexible linker.
aaacgtcgtcagaccagcgcaaccgatttttatcatagcaaacgtcgcctgatttttagcaaaccgcgtcgtccgtatcgtgttgttcgtccgctgggtattgccggtggtggtggttgtgcagcagcatgtgcagcctgtggtagcggtcagcgtgttgcagcacatattaccggcacccgtggtcgtagcaataccctgagcagcccgaatagcaaaaatgaaaaagcactgggtcgcaaaattaacagctgggaaagcagccgtagtggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 77 - protein, 14 aa, Artificial Sequence
  synthesized, an artificial sequence of a steric linker
GGGCAAACAACGSG

SEQ ID NO: 78 - protein, 281 aa, Homo sapiens [sequence omitted]

(19) United States
(12) Patent Application Publication
(10) Pub. No.: US 2014/0031283 A1
(43) Pub. Date: Jan. 30, 2014

(54) ANTICANCER FUSION PROTEIN

(71) Applicants: Jerzy Szczepan Pieczykolan, Radecznica (PL); Sebastian Dominik Pawlak, Warszawa (PL); Barttomie Maciej Aerek, Dabrowa (PL); Piotr Kamil Rózga, Skierniewice (PL); Urszula Marta Szawlowska, Warszawa (PL)

(72) Inventors: Jerzy Szczepan Pieczykolan, Radecznica (PL); Sebastian Dominik Pawlak, Warszawa (PL); Barttomie Maciej Aerek, Dabrowa (PL); Piotr Kamil Rózga, Skierniewice (PL); Urszula Marta Szawlowska, Warszawa (PL)

(73) Assignee: ADAMED SP. Z O.O., Czosnów k/Warszawy (PL)

(21) Appl. No.: 14/008,234

(22) PCT Filed: Apr. 19, 2012
(86) PCT No.: PCT/EP12/57219

§ 371 Date: Sep. 27, 2013

(30) Foreign Application Priority Data
Apr. 19, 2011 (PL) PL394618

(51) Int. Cl. C07K 14/52; C07K 14/435; C07K 14/47

(52) U.S. Cl. CPC C07K 14/52; C07K 14/4715; C07K 14/435
USPC 514/7.6; 530/399

(57) ABSTRACT

A fusion protein comprising domain (a) which is the functional fragment of a hTRAIL protein sequence, which fragment begins with an amino acid at a position not lower than hTRAIL95, or a homolog of said functional fragment having at least 70% sequence identity; and at least one domain (b) which is the sequence of an effector peptide having anti-proliferative activity against tumour cells, wherein the sequence of domain (b) is attached at the C-terminus or at the N-terminus of domain (a). The fusion protein can be used for the treatment of cancer diseases.

The invention relates to the field of therapeutic fusion proteins, especially recombinant fusion proteins. More particularly, the invention relates to fusion proteins comprising the fragment of a sequence of the soluble human TRAIL protein and a sequence of an antiproliferative peptide, pharmaceutical compositions containing them, their use in therapy, especially as anticancer agents, and to polynucleotide sequences encoding the fusion proteins, expression vectors containing the polynucleotide sequences, and host cells containing these expression vectors.

TRAIL protein, a member of the cytokines family (Tumor Necrosis Factor-Related Apoptosis Inducing Ligand), also known as Apo2L (Apo2-ligand), is a potent activator of apoptosis in tumor cells and in cells infected by viruses. TRAIL is a ligand naturally occurring in the body. TRAIL protein, its amino acid sequence, coding DNA sequences and protein expression systems were disclosed for the first time in EP0835305A1.

TRAIL protein exerts its anticancer activity by binding to pro-apoptotic surface TRAIL receptors 1 and 2 (TRAIL-R1/R2) and subsequent activation of these receptors. These receptors, also known as DR4 and DR5 (death receptor 4 and death receptor 5), are members of the TNF receptor family and are overexpressed by different types of cancer cells. Activation of these receptors can induce external signaling pathway of suppressor gene p53-independent apoptosis, which by activated caspase-8 leads to the activation of executive caspases and thereby degradation of nucleic acids. Caspase-8 released upon TRAIL activation may also cause the release of Bid protein and thereby indirect activation of mitochondrial pathway, Bid protein being translocated to mitochondria, where it stimulates the release of cytochrome c, thus indirectly amplifying the apoptotic signal from death receptors.

TRAIL acts selectively on tumor cells essentially without inducing apoptosis in healthy cells which are resistant to this protein. Therefore, the enormous potential of TRAIL was recognized as an anticancer agent which acts on a wide range of different types of tumor cells, including hematologic malignancies and solid tumors, while sparing normal cells and exerting potentially relatively little side effects.

TRAIL protein is a type II membrane protein having the length of 281 amino acids, and its extracellular region comprising amino acid residues 114-281 upon cleavage by proteases forms soluble sTRAIL molecule of 20 kDa size, which is also biologically active. Both TRAIL and sTRAIL forms are capable of triggering apoptosis via interaction with TRAIL receptors present on target cells. Strong antitumor activity and very low systemic toxicity of soluble part of TRAIL molecule was demonstrated using cell lines tests. Also, human clinical studies with recombinant human soluble TRAIL (rhTRAIL) having amino acid sequence corresponding to amino acids 114-281 of hTRAIL, known under the INN dulanermin, showed its good tolerance and absence of dose limiting toxicity.

Fragment of TRAIL shorter than 114-281 is also able to bind with membrane death receptors and induce apoptosis via these receptors, as recently reported for recombinant circularly permuted mutant of 122-281hTRAIL for example in EP 1 688 498.

Toxic effects of recombinant TRAIL protein on liver cells reported up to now appear to be associated with the presence of modification, i.e. polyhistidine tags, while untagged TRAIL showed no systemic toxicity.

However, in the course of further research and development it appeared that many cancer cells showed primary or acquired resistance to TRAIL (see for example WO2007/022214). Although the mechanism of resistance to TRAIL has not been fully understood, it is believed that it may manifest itself at different levels of TRAIL-induced apoptosis pathway, ranging from the level of cell surface receptors to the executive caspases within the signaling pathway. This resistance limits the usefulness of TRAIL as an anticancer agent.

Furthermore, in clinical trials on patients the actual effectiveness of TRAIL as a monotherapy proved to be low. To overcome this low efficiency and the resistance of tumors to TRAIL, various combination therapies with radio- and chemotherapeutic agents were designed, which resulted in synergistic apoptotic effect (WO2009/002947; A. Almasan and A. Ashkenazi, Cytokine Growth Factor Reviews 14 (2003) 337-348; R K Srivastava, Neoplasis, Vol 3, No. 6, 2001, 535-546, Soria J C et al., J. Clin. Oncology, Vol 28, No. 9 (2010), p. 1527-1533). The use of rhTRAIL for cancer treatment in combination with selected conventional chemotherapeutic agents (paclitaxel, carboplatin) and monoclonal anti-VEGF antibodies are described in WO2009/140469. However, such a combination necessarily implies well-known deficiencies of conventional chemotherapy or radiotherapy.

Moreover, the problem connected with TRAIL therapy has proved to be its low stability and rapid elimination from the body after administration.

Constructed fusion protein containing sequences of angiogenesis inhibitor vasostatin and TRAIL114-281 linked with a metalloprotease cleavage site linker was described as exhibiting apoptosis-inducing effect in tumor cells by A. I. Guo et al in Chinese Journal of Biochemistry and Molecular Biology 2008, vol. 24(10), 925-930.

Constructed fusion protein containing sequences of angiogenesis inhibitor calreticulin and TRAIL114-281 was described as exhibiting apoptosis-inducing effect in tumor cells in CN1609124A.

CN 1256347C discloses fusion protein composed of kininogen D5 60-148 and TRAIL 114-281.

Constructed fusion protein containing sequences of angiogenesis inhibitor kininostatin, vasostatin and canstatin attached to N- or C-terminus of TRAIL114-281 linked with linker encoding GGGSGGSG are mentioned in Feng Feng-Yi “Phase I and Clinical Trial of Rh-Apo2L and Apo2L-Related Experimental Study”, Ph.D. degree thesis, Chinese Peking Union Medical, 2006-10-01; http: //www.lw23.com/lunwen_{—}957708432.

Constructed fusion protein containing sequences Tumstatin 183-230 of an angiogenesis inhibitor tumstatin and TRAIL114-281 was described as exhibiting induction of apoptosis of pancreatic cancer cells by N. Ren et al in Academic Journal of Second Military Medical University 2008, vol. 28(5), 676-478.

US2005/244370 and corresponding WO2004/035794 disclose the construct of TRAIL95-281 as an effector domain linked by a peptide linker with extracellular part of another member of TNF family ligands CD40 as a cell surface binding domain. It is stated that activation of the construct is via binding of its CD40 part.

Shin J. N. et al., Experimental Cell Research, vol. 312, no. 19, 2006, p. 3892-3898), disclosed constructed fusions proteins of sTRAIL and IL-18 with a matrix metalloproteinase cleavage site introduced at the connecting site as a proform of TRAIL that can be activated and released in the areas where metalloproteinases are pathologically produced, such as tumor environment. Constructs of sTRAIL with IFN-gamma and endostatin were also produced but neither characterized nor tested.

One of the objectives in cancer therapy is the inhibition of tumor cells proliferation (growth). Cells with acquired malignant phenotype (due to mutation, activities of carcinogens or disorders of DNA repair) lose their ability to proper differentiation and acquire the ability to infiltrate. The clones of tumor cells transcribe mainly genes that are associated with rapid growth and invasiveness, and tumor cells are characterized, among others, by disturbances in the control of proliferation.

Beneficial effect of inhibition of tumor cells proliferation in cancer therapy is known. Attempts are made of the clinical use of substances that inhibit or regulate the process of proliferation, both as a cancer therapy and an adjunct cancer therapy.

Inhibition of tumor cell proliferation can be achieved in various ways, such as for example described in the review article “Hallmarks of Cancer: The Next Generation” (Cell, 2011, 646-674). There are known antiproliferative proteins used in anticancer therapies—such as trastuzumab—a monoclonal antibody blocking HER2 used in breast cancer patients with HER2 overexpression. There is also known an antiproliferative activity of many proteins that have not yet been found to be clinically useful in the treatment of human cancers.

For example, antiproliferative activity of human fetoprotein and its fragments is well known. Detailed studies of the properties of individual protein domains revealed the presence of structures located within the 34-amino acid region that is responsible for the growth inhibition of estradiol dependent cells (Mizejewski et al, Mol. Cell. Endocrinol., 18:15-23, 1996). Carboxylic terminus of this region, comprised of eight consecutive amino acids, is the most important fragment, and is able alone to inhibit the growth of cancer cells (Mizejewski G., Cancer Biotherapy [symbol] Radiopharmaceuticals, 22: 73-98, 2007).

Antiproliferative properties of p21WAF1 protein are also known. Short peptides based on the amino acid sequence of p21WAF1 exerting comparable potential to bind and inhibit D1-CDK4 complex and thus stop the cell cycle in G1 phase were synthesized (Ball et al, Current Biology, 7:71-80, 1996).

It is also known that protein DOC-2/DAB2 (Differentially expressed in Ovarian Cancer-2/Disabled 2) is a powerful inhibitor of proliferation of prostate cancer cells. It acts by suppressing MAPK kinase transmission pathways by binding to a number of their respective sub elements (c-Src, Grb2) (Zhou et al, J Biol Chem 276: 27793-27798, 2001, Zhou et al, J Biol Chem, 278: 6936-6941, 2003). Its essential component is a proline-rich domain present at the carboxy-terminal DOC-2/DAB2 (Zhou et al, Cancer Res, 66: 8954-8958, 2006).

Inhibition of CDK4-cyclin binding by the p16 protein or a fragment thereof is commonly regarded as a suppressor of neoplasia (Fahraeus et al, Oncogene, 16: 587-596, 1998).

There is also known influence of kinase ERK on the degree of tumor cell proliferation (Handra-Luca A., et al, American Journal of Pathology. 2003; 163: 957-967). It is known that a peptide fragment of MEK-1 protein is a selective ERK kinase substrate, and thus it can serve as its selective inhibitor (Bradley R. et al, The Journal of Biological Chemistry, 2002, 277, 8741-8748).

It is also known that selective inhibition of Akt kinase activity leads to inhibition of cell proliferation and tumor cell death (Hennessy B. T, et al, Nature Reviews Drug Discovery 2005, 4, 988-1004).

There are also known antiproliferative properties of Phe-Trp-Leu-Arg-Phe-Thr hexapeptide, consisting in inhibition of the association of E2F and DP and direct inhibition of E2F binding to DNA (Janin Y. L., Amino Acids, 25: 1-40, 2003).

Inhibition of tubulin fibers depolimerisation, preventing sister chromatid separation in mitosis and causing disorders in the migration of chromosomes also results in disorders of the proliferation process (Xiao et al., J. Cell Mol. Med., 2010).

Synergistic effect of melittin protein with the activity of TRAIL protein was shown (Wang et al., JBC Journal of Biological Chemistry, 284, 3804-3813).

Inhibition of telomerase activity and accumulation in the mitochondrial membrane by proteins which are fragments of bee defensin and their analogs is also known (Iwasaki et al., Biosci. Biotechnol. Biochem., 73:683-687, 2009).

It is also known that lasioglossins, positively charged peptides isolated from the venom of bee Lasioglossum laticeps, exert cytotoxic activity against tumor cells (Cerovskŷ et al., Chembiochem, 2009, 10: 2089-2099).

It is also known that inhibition of RasGAP-Aurora B interactions by e.g. protein aptamers from the SH3 domain, exert inhibitory influence on the proliferation of cancer cells (Pamonsinlapatham P. et al., PLoS ONE 3 (8): e2902, 2008).

The impact of inhibition of cell cycle -dependent kinases e.g. kinase CDK 4, for example with p16 peptide, which is the fragment of p16INK4A gene product, is known as well (Derossi D, et al., J Biol Chem. 269:10444-10450, 1994).

There are also known antiproliferative properties of Pep27 protein, the binding of which by cellular receptors results in phosphorylation of a histidine kinase, which causes dephosphorylation of the effector factor VncR and consequently leads to inhibition of autocatalytic pathways and cell death (Dong Gun Lee et al., Cancer Cell International 2005, 5:21).

Many of the antiproliferative substances are currently at different stages of investigations, including clinical trials. However, known therapies aimed at inhibiting proliferation have many well-known disadvantages. For example, there are adverse effects such as thromboembolic complications, haemoptysis and lungs bleeding. Many antiproliferative drugs show also poor bioavailability and toxic side effects.

Safety of anti-antiproliferative drugs is of special importance because of prolonged use and lack of selectivity of therapy. Strong need for an effective therapeutic agent and the nature of oncological diseases necessitate a simplified registration procedure for such group of drugs, therefore it is impossible to know all the side effects and disadvantages of the drug. Although, contrary to the chemotherapeutics, which are directed to all fast proliferating cells, peptide antiproliferative drugs are directed at protein kinases and phosphatases responsible for triggering cascades of phosphorylation and dephosphorylation of proteins or at their substrates or other proteins engaged in proper course of the cell cycle, which results in some reduction of the toxicity of therapy. However, still anticancer therapy directed at inhibiting proliferation while ensuring selectivity against tumor cells is not known. There is therefore a need for new anti-proliferative anticancer therapies with improved toxicological characteristics.

The present invention provides a solution of this problem by providing novel fusion proteins that comprise a domain derived from TRAIL and a short effector peptide domain having antiproliferative activity and not including TRAIL fragments, wherein the effector peptide potentiates or complements the action of TRAIL.

Proteins according to the invention are directed selectively to cancer cells, where the elements of the protein exert their effects, in particular the effector peptide inhibits tumor cells proliferation. Delivery of the proteins of the invention into the tumor environment allows to minimize toxicity against healthy cells in the body as well as side effects and to reduce the frequency of administration. In addition, targeted therapy with the use of proteins according to the invention allows to avoid the problem of low efficiency of previously known nonspecific antiproliferative therapies caused by high toxicity and by necessity of administering high doses.

It turned out that in many cases fusion proteins of the invention are more potent than soluble TRAIL and its variants including a fragment of the sequence. Until now, known effector peptides used in the fusion protein of the invention have not been used in medicine as such because of unfavorable kinetics, rapid degradation by nonspecific proteases or accumulation in the body caused by lack of proper sequence of activation of pathways, which is necessary to enable the proper action of the effector peptide at target site. Incorporation of the effector peptides into the fusion protein allows their selective delivery to the site where their action is desirable. Furthermore, the attachment of the effector peptide increases the mass of protein, resulting in prolonged half-life and increased retention of protein in the tumor and its enhanced efficiency. Additionally, in many cases, novel fusion proteins also overcome natural or induced resistance to TRAIL.

### DESCRIPTION OF FIGURES

The invention will now be described in detail with reference to the Figures of the drawing.

FIG. 1 presents a schematic structure of fusion proteins of the invention according to Ex. 1, Ex. 2, Ex. 3, Ex. 4 and Ex. 5.

FIG. 2 presents a schematic structure of fusion proteins of the invention according to Ex. 6, Ex. 7, Ex. 8, Ex. 8A, Ex. 9 and Ex. 10.

FIG. 3 presents a schematic structure of fusion proteins of the invention according to Ex. 11, Ex. 12, Ex. 13, Ex. 14, and Ex. 15.

FIG. 4 presents a schematic structure of fusion proteins of the invention according to Ex. 16, Ex. 17, Ex. 18, Ex. 19, and Ex. 20.

FIG. 5 presents a schematic structure of fusion proteins of the invention according to Ex. 21, Ex. 22, Ex. 23, Ex. 24, and Ex. 25.

FIGS. 6A and 6B show circular dichroism spectra for rhTRAIL95-281 and fusion proteins of Ex. 1^{a} and Ex. 2^{a} (FIG. 6A), and Ex. 8^{a} and rhTRAIL114-281 (FIG. 6B) expressed in specific ellipticity.

FIG. 7 presents tumor volume changes (% of initial stage) in Crl:CD1-Foxn1nu mice burdened with colon cancer HCT116 treated with fusion protein of the invention of Ex. 2^{a} compared to rhTRAIL114-281

FIG. 8 presents the tumor growth inhibition values (% TGI) in Crl:CD1-Foxn1^{nu} 1 mice burdened with colon cancer HCT116 treated with fusion protein of the invention of Ex. 2^{a} compared to rhTRAIL114-281.

FIG. 9 presents tumor volume changes (% of initial stage) in Crl:CD1-Foxn1nu mice burdened with lung cancer NCI-H460-Luc2 treated with fusion protein of the invention of Ex. 2^{a} compared to rhTRAIL114-281.

FIG. 10 presents the tumor growth inhibition values (% TGI) in Crl:CD1-Foxn1^{nu} 1 mice burdened with lung cancer NCI-H460-Luc2 treated with fusion protein of the invention of Ex. 2^{a} compared to rhTRAIL114-281.

FIG. 11 presents tumor volume changes (% of initial stage) in Crl:SHO-Prkdc^{scid}Hr^{hr} mice burdened with colon cancer HCT116 treated with fusion protein of the invention of Ex. 8^{a} compared to rhTRAIL114-281.

FIG. 12 presents the tumor growth inhibition values (% TGI) in Crl:SHO-Prkdc^{scid}Hr^{hr} mice burdened with colon cancer HCT116 treated with fusion protein of the invention of Ex. 8^{a} compared to rhTRAIL114-281.

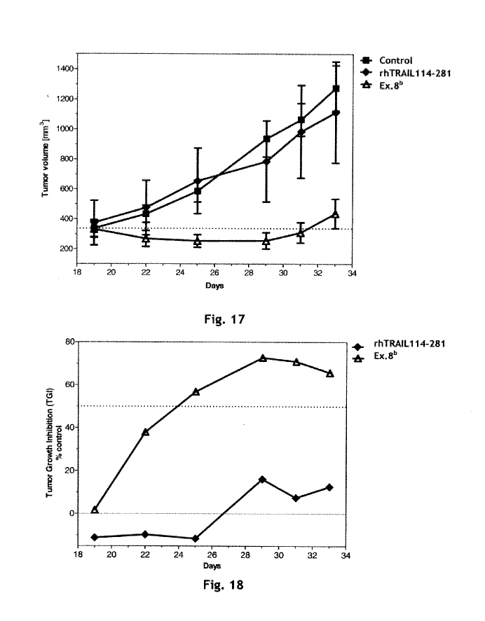

FIG. 11a presents tumor volume changes (% of initial stage) in Crl:SHO-Prkdc^{scid}Hr^{hr} mice burdened with colon cancer HCT116 treated with fusion protein of the invention of Ex. 8^{b} compared to rhTRAIL114-281.

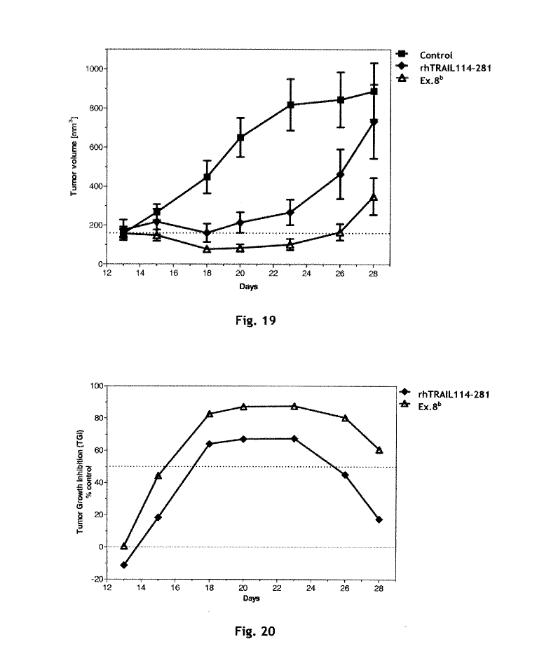

FIG. 12a presents the tumor growth inhibition values (% TGI) in Crl:SHO-Prkdc^{scid}Hr^{hr} mice burdened with colon cancer HCT116 treated with fusion is protein of the invention of Ex. 8^{b} compared to rhTRAIL114-281.

FIG. 13 presents tumor volume changes (% of initial stage) in Crl:SHO-Prkdc^{scid}Hr^{hr} mice burdened with colon cancer SW620 treated with fusion protein of the invention of Ex. 8^{b} compared to rhTRAIL114-281.

FIG. 14 presents the tumor growth inhibition values (% TGI) in Crl:SHO-Prkdc^{scid}Hr^{hr} mice burdened with colon cancer SW620 treated with fusion protein of the invention of Ex. 8^{b} compared to rhTRAIL114-281.

FIG. 15 presents tumor volume changes (% of initial stage) in Crl:SHO-Prkdc^{scid}Hr^{hr} mice burdened with colon cancer Colo205 treated with fusion protein of the invention of Ex. 8^{b} compared to rhTRAIL114-281.

FIG. 16 presents the tumor growth inhibition values (% TGI) in Crl:SHO-Prkdc^{scid}Hr^{hr} mice burdened with colon cancer Colo205 treated with fusion protein of the invention of Ex. 8^{b} compared to rhTRAIL114-281.

FIG. 17 presents tumor volume changes (% of initial stage) in Crl:SHO-Prkdc^{scid}Hr^{hr} mice burdened with liver cancer HepG2 treated with fusion protein of the invention of Ex. 8^{b} compared to rhTRAIL114-281.

FIG. 18 presents the tumor growth inhibition values (% TGI) in Crl:SHO-Prkdc^{scid}Hr^{hr} mice burdened with liver cancer HepG2 treated with fusion protein of the invention of Ex. 8^{b} compared to rhTRAIL114-281.

FIG. 19 presents tumor volume changes (% of initial stage) in Crl:SHO-Prkdc^{scid}Hr^{hr} mice burdened with lung cancer NCI-H460 treated with fusion protein of the invention of Ex. 8^{b} compared to rhTRAIL114-281.

FIG. 20 presents the tumor growth inhibition values (% TGI) in Crl:SHO-Prkdc^{scid}Hr^{hr} mice burdened with lung cancer NCI-H460 treated with fusion protein of the invention of Ex. 8^{b} compared to rhTRAIL114-281.

### DETAILED DESCRIPTION OF THE INVENTION

The invention relates to a fusion protein comprising:
domain (a) which is the functional fragment of a sequence of soluble hTRAIL protein, which fragment begins with an amino acid at a position not lower than hTRAIL95 or a homolog of said functional fragment having at least 70% sequence identity, and
at least one domain (b) which is the sequence of an effector peptide having anti-proliferative activity against tumor cells,

wherein the sequence of the domain (b) is attached at the C-terminus and/or N-terminus of domain (a).

The term “the functional soluble fragment of a sequence of soluble hTRAIL” should be understood as denoting any such fragment of soluble hTRAIL that is capable of inducing apoptotic signal in mammalian cells upon binding to its receptors on the surface of the cells.

It will be also appreciated by a skilled person that the existence of at least 70% homology of the TRAIL sequence is known in the art.

It should be understood that domain (b) of the effector peptide in the fusion protein of the invention is neither hTRAIL protein nor a part or fragment of hTRAIL protein.

The term “peptide” in accordance with the invention should be understood as a molecule built from plurality of amino acids linked together by means of a peptide bond. Thus, the term “peptide” according to the invention includes oligopeptides, polypeptides and proteins.

In the present invention the amino acid sequences of peptides will be presented in a conventional manner adopted in the art in the direction from N-terminus (N-end) of the peptide towards its C-terminus (C-end). Any sequence will thus have its N-terminus on the left side and C-terminus on the right side of its linear presentation.

The fusion protein of the invention incorporates at least one domain (b) of the effector peptide, attached at the C-terminus and/or or at the N-terminus of domain (a).

In a particular embodiment, domain (a) is the fragment of hTRAIL sequence, beginning with an amino acid from the range of hTRAIL95 to hTRAIL121, inclusive, and ending with the amino acid hTRAIL 281.

In particular, domain (a) may be selected from the group consisting of sequences corresponding to hTRAIL95-281, hTRAIL114-281, hTRAIL119-281, hTRAIL120-281 and hTRAIL121-281. It will be evident to those skilled in the art that hTRAIL95-281, hTRAIL114-281, hTRAIL119-281, hTRAIL120-281 and hTRAIL121-281 represent a fragment of human TRAIL protein starting with amino acid marked with the number 95, 114, 119, 120 and 121, respectively, and ending with the last amino acid 281, in the known sequence of hTRAIL published in GenBank under Accession No. P50591.

In another particular embodiment, domain (a) is a homolog of the functional fragment of soluble hTRAIL protein sequence beginning at amino acid position not lower than hTRAIL95 and ending at amino acid hTRAIL281, the sequence of which is at least in 70%, preferably in 85%, identical to original sequence.

In specific variants of this embodiment domain (a) is a homolog of the fragment selected from the group consisting of sequences corresponding to hTRAIL95-281, hTRAIL114-281, hTRAIL116-281, hTRAIL120-281 and hTRAIL121-281.

It should be understood that a homolog of the hTRAIL fragment is a variation/modification of the amino acid sequence of this fragment, wherein at least one amino acid is changed, including 1 amino acid, 2 amino acids, 3 amino acids, 4 amino acids, 5 amino acids, 6 amino acids, and not more than 15% of amino acids, and wherein a fragment of the modified sequence has preserved functionality of the hTRAIL sequence, i.e. the ability of binding to cell surface death receptors and inducing apoptosis in mammalian cells. Modification of the amino acid sequence may include, for example, substitution, deletion and/or addition of amino acids.

Preferably, the homolog of hTRAIL fragment having modified sequence shows a modified affinity to the death receptors DR4 (TRAIL-R1) or DR5 (TRAIL-R2) in comparison with the native fragment of hTRAIL.

The term “modified affinity” refers to an increased affinity and/or affinity with altered receptor selectivity.

Preferably, the homolog of the fragment of hTRAIL having modified sequence shows increased affinity to the death receptors DR4 and DR5 compared to native fragment of hTRAIL.

Particularly preferably, the homolog of fragment of hTRAIL having modified sequence shows increased affinity to the death receptor DR5 in comparison with the death receptor DR4, i.e. an increased selectivity DR5/DR4.

Also preferably, the homolog of fragment of hTRAIL having modified sequence shows an increased selectivity towards the death receptors DR4 and/or DR5 in relation to the affinity towards the receptors DR1 (TRAIL-R3) and/or DR2 (TRAIL-R4).

Modifications of hTRAIL resulting in increased affinity and/or selectivity towards the death receptors DR4 and DR5 are known to those skilled in the art, for example from the publication Tur V, van der Sloot A M, Reis C R, Szegezdi E, Cool R H, Samali A, Serrano L, Quax W J. DR4-selective tumor necrosis factor-related apoptosis-inducing ligand (TRAIL) variants obtained by structure-based design. J. Biol. Chem. 2008 Jul. 18; 283(29):20560-8, which describes the D218H mutation having increased selectivity towards DR4, or Gasparian M E, Chernyak B V, Dolgikh D A, Yagolovich A V, Popova E N, Sycheva A M, Moshkovskii S A, Kirpichnikov M P. Generation of new TRAIL mutants DR5-A and DR5-B with improved selectivity to death receptor 5, Apoptosis. 2009 June; 14(6):778-87, which describes the D269H mutation having a reduced affinity towards DR4. hTRAIL mutants resulting in increased affinity towards one receptor selected from the DR4 and DR5 comparing with DR1 and DR2 receptors and increased affinity towards the receptor DR5 comparing with DR4 are also described in WO2009077857 and WO2009066174.

Suitable mutations are one or more mutations in the positions of native hTRAL selected from the group consisting of amino acid 131, 149, 159, 193, 199, 201, 204, 204, 212, 215, 218 and 251, in particular, mutations involving the substitution of an amino acid with a basic amino acid such as lysine, histidine or arginine, or amino acid such as glutamic acid or aspargic acid. Particularly one or more mutations selected from the group consisting of G131R, G131K, R149I, R149M, R149N, R149K, S159R, Q193H, Q193K, N199H, N199R, K201H, K201R, K204E, K204D, K204L, K204Y, K212R, S215E, S215H, S215K, S215D, D218Y, D218H, K251D, K251E and K251Q, as described in WO2009066174, may be specified.

Suitable mutations are also one or more mutations in the positions of native hTRAIL selected from the group consisting of amino acid 195, 269 and 214, particularly mutations involving the substitution of an amino acid with a basic amino acid such as lysine, histidine or arginine. Particularly one or more mutations selected from the group consisting of D269H, E195R, and T214R, as described in WO2009077857, may be specified.

In a particular embodiment, the domain (a) which is a homolog of the fragment of hTRAIL is selected from D218H mutant of the native TRAIL sequence, as described in WO2009066174, or the Y189N-R191K-Q193R-H264R-I266R-D269H mutant of the native TRAIL sequence, as described in Gasparian M E et at. Generation of new TRAIL mutants DR5-A and DR5-B with improved selectivity to death receptor 5, Apoptosis. 2009 June; 14(6): 778-87.

According to the invention, the fusion protein comprises as the effector peptide an anti-proliferative peptide, which has anti-proliferative activity against tumor cells, i.e. inhibiting effect on tumor cells proliferation.

It should be understood that “tumor cells proliferation” relates to the step of cell division and growth in a tumor cell cycle and the effector peptide has the anti-proliferative activity with respect to the growth of tumor cells as such.

Therefore, “tumor cells proliferation” inhibiting effect does not encompass inhibiting proliferation of endothelial cells as a step of angiogenesis. Effector peptides having anti-angiogenic activity, i.e. activity of inhibiting growth of endothelial cells are therefore excluded from the scope of the effector peptides according to the invention.

Specifically, effector peptides selected from the group consisting of calreticulin, tumstatin 183-230, kininogen D5, vasostatin, kininostatin, endostatin and canstatin are not encompassed by the invention.

According to the invention, the effector peptide can exert its antiproliferative effect against tumor cells in different ways, such as for example selected from the following group:
suppression of MAPK kinases (mitogen-activated protein kinases) transmission pathways, for example by blocking FGF-2 receptor (basic fibroblast growth factor 2 receptor, also known as bFGF-, FGF2- or FGF-B receptor) or DD2 peptide derived from DAB2 protein;
inhibition of growth of estradiol dependent cells, for example by human fetoprotein or its fragment;
stopping cell-cycle in G1 phase, such as by inhibition of cyclin D1-CDK4 (cyclin-dependent kinase 4) complex;
enzymatic breakdown of arginine, such as by arginine deiminase from Mycoplasma arginini;
inhibition of cell-cycle kinases, such as inhibition of CDK4/5/6 kinase (cyclin-dependent kinases), or inhibition of ERK kinases (extracellular-signal-regulated kinases) activation, or inhibition of Akt kinase (also known as Protein Kinase B (PKB), a serine/threonine-specific protein kinase) coactivation;
inhibition of transcription factor E2F (transcription factors (TF) in higher eukaryotes) association with DP proteins (also known as transcription factor DP, E2F dimerisation partner);
inhibition of tubulin fibres association/polymerization;
inhibition of telomerase activity;
inhibition of RasGAP (GTPase-activator protein for Ras-like GTPases)-Aurora B kinase interactions or histidine kinase activation; and
disturbing ionic balance across the cell membrane.

In one embodiment of the invention the effector peptide of domain (b) may be a peptide capable of suppressing MAPK kinases transmission pathways. An example is an analogue of binding domain of FGF-2 receptor which is responsible for the blockade of FGF-2 receptor and in consequence inhibition of tumor growth. In particular, such an effector peptide can be a 16-amino acid peptide presented by SEQ. No. 26 in the attached Sequence Listing.

Another effector peptide of this embodiment of the invention can be a fragment of DOC-2/DAB2 protein. In particular, such an effector peptide can be an 18-amino acid peptide DD2—a proline-rich domain present on the carboxy terminus of DOC-2/DAB2, presented by SEQ. No. 30 in the attached Sequence Listing, which participates in suppression of transmission pathways of MAPK kinases by binding to a number of their respective sub elements (c-Src, Grb2).

In another embodiment of the invention the effector peptide of domain (b) may be a peptide capable of inhibition of growth of estradiol dependent cells, for example human fetoprotein or its fragment. In particular, such an effector peptide can be a 34-amino acid fragment of human alpha-fetoprotein presented by SEQ. No. 27 in the attached Sequence Listing. Another effector peptide of this embodiment can be an 8-amino acid fragment of human alpha-fetoprotein, localized on C-terminal fragment of SEQ. No. 27, and presented by SEQ. No. 28 in the attached Sequence Listing.

In another embodiment of the invention the effector peptide of domain (b) may be a peptide capable of stopping cell-cycle in G1 phase, such as by inhibition of cyclin D1-CDK4 complex. In particular, such an effector peptide can be a trojan p16 peptide, or its fragment, inhibiting the activity of kinases CDK4 and CDK6. In particular, such an effector peptide—a fragment of p16INK4A gene product—is presented by SEQ. No. 32 in the attached Sequence Listing. Such an effector peptide can be also another fragment of trojan p16 peptide—a fragment of p16INK4A gene product fused with a 17-amino-acid transporting domain of antennapedia (Derossi D, A H Joliot, G Chassaings, A Prochiantz, J Biol Chem. 269:10444-10450,1994), presented as SEQ. No. 33 in the attached Sequence Listing.

In another embodiment of the invention the effector peptide of domain (b) may be a peptide capable of enzymatic breakdown of arginine, such as by arginine deiminase from Mycoplasma arginini. In particular, such an effector peptide is presented by SEQ. No. 31 in the attached Sequence Listing.

In another embodiment of the invention the effector peptide of domain (b) may be a peptide capable of inhibition of cell-cycle kinases, such as a CDK4/5 inhibitor. In particular, such an effector peptide can be a fragment of p21WAF1 protein, such as a 20-amino acid fragment of p21WAF1 protein presented by SEQ. No. 29 in the attached Sequence Listing.

Another effector peptide of this embodiment can be a peptide—inhibitor of ERK activation. In particular, such an effector peptide can be a fragment of MEK-1 protein, such as presented by SEQ. No. 34 in the attached Sequence Listing.

Another effector peptide of this embodiment can be a peptide—coactivator of Akt kinase. In particular, such an effector peptide—an N-terminal fragment of PH domain of TCL1 protein—is presented by SEQ. No. 35 in the attached Sequence Listing.

In another embodiment of the invention the effector peptide of domain (b) may be a peptide capable of inhibition of transcription factor E2F association with DP protein. In particular, such an effector peptide—a hexapeptide Phe-Trp-Leu-Arg-Phe-Thr—is presented by SEQ. No. 36 in the attached Sequence Listing. Another effector peptide of domain (b) can be a peptide being an analogue of FGF-2 binding domain. In particular, such an effector peptide—a 8 amino acid peptide blocking FGF-2 receptor—is presented by SEQ. No. 41 in the attached Sequence Listing.

In another embodiment of the invention the effector peptide of domain (b) may be a peptide capable of inhibition of tubulin fibres association/polymerization. Such an effector peptide can be a fragment of tubulin responsible for forming of heterodimers structures, contributing to inhibition of tubulin fibers polymerisation. In particular, such an effector peptide—a 13-amino acid fragment of tubulin—is presented by SEQ. No. 37 in the attached Sequence Listing, and another effector peptide—a 10-amino acid fragment of tubulin—is presented by SEQ. No. 38 in the attached Sequence Listing.

In another embodiment of the invention the effector peptide of domain (b) may be a peptide capable of inhibition of telomerase activity. Such an effector peptide can be a peptide based on the sequence of a bee defensin responsible for telomerase activity inhibition. In particular, such an effector peptide—a 6 amino acid C2 peptide based on the sequence of a bee defensin—is presented by SEQ. No. 40 in the attached Sequence Listing. Another effector peptide of this embodiment can be a peptide lasioglossin present in the bee venom. In particular, such an effector peptide—lasioglossin LL-2—is presented by SEQ. No. 42 in the attached Sequence Listing.

In another embodiment of the invention the effector peptide of domain (b) may be a peptide capable of inhibition of RasGAP-Aurora B interactions or histidine kinase activation. In particular, such an effector peptide—a 13-amino acid peptid binding 5H3 domain of RasGAP—is presented by SEQ. No. 43 in the attached Sequence Listing. Another effector peptide of this embodiment can be a peptide which after binding by cell receptors causes histidine kinase phosphorylation, which in turn leads to effector factor VncR dephosphorylation. In particular, such an effector peptide—an analogue of Pep27 peptide—is presented by SEQ. No. 44 in the attached Sequence Listing.

In another embodiment of the invention the effector peptide of domain (b) may be a peptide capable of disturbing ionic balance across the cell membrane. In particular, such an effector peptide melittin—is presented by SEQ. No. 39 in the attached Sequence Listing.

In the specific embodiments of the fusion protein of the present invention, the effector peptide is selected from the group consisting of:
SEQ. No.26 (16-amino acids peptide blocking FGF-2 receptor),
SEQ. No.27 (a fragment of alpha-fetoprotein),
SEQ. No.28 (a C-terminal fragment of alpha-fetoprotein),
SEQ. No.29 (a fragment of p21^{WAF1} protein),
SEQ. No.30 (a DD2 peptide from DAC-2/DAB-2 protein),
SEQ. No.31 (an arginine deiminase),
SEQ. No.32 (a fragment of p16 peptide),
SEQ. No.33 (a fragment of p16 peptide fused with a 17-amino-acid transporting domain of antennapedia),
SEQ. No.34 (a fragment of MEK-1),
SEQ. No.35 (a fragment of PH domain of TCL1 protein),
SEQ. No.36 (a hexapeptide inhibitor of E2F),
SEQ. No.37 (an inhibitor of tubulin polymerisation),
SEQ. No.38 (an inhibitor of tubulin polymerisation),
SEQ_{’} No.39 (melittin),
SEQ. No.40 (synthetic C2 telomerase inhibitor),
SEQ. No.41 (an 8-amino acids inhibitor of interactions with FGF-2R),
SEQ. No.42 (lassioglossin LL-2),
SEQ. No.43 (an inhibitor of Aurora RG27 kinase), and
SEQ. No.44 (an analog of Pep27).

Upon binding to TRAIL receptors present on the surface of cancer cells, the fusion protein will exert a double effect. Domain (a), that is a functional fragment of TRAIL or its homolog with preserved functionality, will exert its known agonistic activity, i.e. binding to death receptors on the cell surface and activation of extrinsic pathway of apoptosis. The effector peptide of the domain (b) of the fusion protein will be able to potentially exert its action intracellularly in parallel to the activity of TRAIL domain by inhibition if proliferation of tumor cells.

If the fusion protein comprises a cleavage sequence recognized by a protease, the effector peptide could previously be cleaved from the fragment of TRAIL by metalloproteinases or urokinases overexpressed in the tumor environment.

In the fusion protein of the invention, antitumor effect of TRAIL could potentially be enhanced by activation of other elements that affect proliferation of cells, such as for example inhibition of growth of estradiol dependent cells, the inhibition of cyclin D1-CDK4 complex, suppression of MAPK kinases transmission pathways, enzymatic breakdown of arginine, CDK4/5/6 kinase inhibition, inhibition of ERK kinase activation, inhibition of Akt kinase coactivation, inhibition of transcription factor E2F association with DP proteins, inhibition of tubulin fibres association, inhibition of telomerase activity, inhibition of RasGAP-Aurora B interactions or histidine kinase activation.

In one of the embodiments of the invention, domain (a) and domain (b) are linked by at least one domain (c) comprising the sequence of a cleavage site recognized by proteases present in the cell environment, especially in the tumor cell environment. The linkage of the domain (a) with the domain (b) by at least one domain (c) means that between domains (a) and (b) more than one domain (c) may be present, in particular one or two domains (c).

The protease cleavage site can be selected from:
a sequence recognized by metalloprotease MMP, in particular (Pro Leu Gly Leu Ala Gly Glu Pro/PLGLAGEP) designated as SEQ. No.45, or (Pro Leu Gly Ile Ala Gly Glu /PLGIAGE) or (Pro Leu Gly Leu Ala Gly GluPro /PLGLAGEP);
a sequence recognized by urokinase uPA, in particular Arg Val Vat Arg (RVVR) designated as SEQ. No. 46 or a fragment thereof, which with the last amino acid of the sequence to which is attached forms SEQ. No.46,

and their combinations.

In one of the embodiments of the invention, the protease cleavage site is a combination of the sequence recognized by metalloprotease MMP and a sequence recognized by urokinase uPA, located next to each other in any order.

In one embodiment, domain (c) is a combination of MMP/uPA, such as SEQ. No. 45/SEQ. No. 46, or a combination of uPA/MMP, such as SEQ. No. 46/SEQ. No. 45.

Proteases metalloprotease MMP and urokinase uPA are overexpressed in the tumor environment. The presence of the sequence recognized by the protease enables the cleavage of domain (a) from domain (b), i.e. the release of the effector domain (b) and thus its activation.

The presence of the protease cleavage site, by allowing quick release of the effector peptide, increases the chances of transporting the peptide to the place of its action before random degradation of the fusion protein by proteases present in the cell occurs.

Additionally, a transporting domain (d) may be attached to domain (b) of the effector peptide of the fusion protein of the invention.

Domain (d) may be for example selected from the group consisting of:

(d1) a polyarginine sequence transporting through the cell membrane, consisting of 6, 7, 8, 9, 10 or 11 Arg residues,

(d2) a fragment of antennapedia protein domain (SEQ. No. 48) as a domain transporting through the cell membrane,

(d3) another fragment of antennapedia protein domain (SEQ. No. 49) as a domain transporting through the cell membrane,

and combinations thereof.

The combination of domains (d1) (d2) and (d3) may comprise, in particular, the combination of (d1)/(d2), (d1)/(d3) or (d1)/(d2)/(d3).

Furthermore, the combination of domains (d1), (d2) and (d3) may include domains located next to each other and connected to one end of domain (b) and/or domains linked to different ends of domain (b).

It should be understood that in the case when the fusion protein has both the transporting domain (d) attached to domain (b) and domain (c) of the cleavage site between domains (a) and (b), then domain (c) is located in such a manner that after cleavage of the construct transporting domain (d) remains attached to domain (b). In other words, if the fusion protein contains both the transporting domain (d) and the cleavage site domain (c), then domain (d) is located between domain (b) and domain (c), or is located at the end of domain (b) opposite to the place of attachment of domain (d).

The invention does not comprise such a variant in which domain (d) is located between domain (c) and domain (a), that is the case when after cleavage of the construct transporting domain remains attached to the TRAIL domain.

Translocation domain constituting a fragment of antennapedia protein domain (SEQ. No. 48) as well as another fragment of antennapedia protein domain(SEQ. No. 49) is capable of translocation through the cell membranes (Derossi D, A H Joliot, G Chassaings, A Prochiantz, J Biol Chem. 269:10444-10450 (1994) and can be used to introduce the effector peptide to the tumor cell compartments.

The sequence (d1) transporting trough the cell membranes may be any sequence known in the art consisting of several arginine residues, translocating the effector peptide trough the cell membrane to the cytoplasm of target cell (D., Hea, H., Yangb, Q., Lina, H., Huang, Arg9-peptide facilitates the internalization of an anti-CEA immunotoxin and potentiates its specific cytotoxicity to target cells, The international Journal of Biochemistry [symbol] Cell Biology 37 (2005) 19Z-205; Shiroh Futaki et al JBC, Vol. 276, No. 8, Issue of Feb. 23, pp. 5836-5840, 2001).

Other useful cell penetrating peptides are described in F. 5aid Hassane et al Cell. Mol. Life Sci. DOI 10.1007/s00018-009-0186-0.

Apart from the main functional elements of the fusion protein and the cleavage site domain(s), the fusion proteins of the invention may contain a neutral sequence/sequences of a flexible steric glycine-cysteine-alanine linker (spacer). Such linkers/spacers are well known and described in the literature. Their incorporation into the sequence of the fusion protein is intended to provide the correct folding of proteins produced by the process of its overexpression in the host cells.

In particular, the flexible steric linker may be SEQ. No.47, which is a combination of cysteine and alanine residues. In another embodiment the flexible steric linker may be a combination of glycine and serine residues such as for example a fragment Gly Gly Gly Ser Gly/GGGSG or any fragment thereof acting as steric linker, for example Gly Gly Gly/GGG.

In other embodiment, the flexible steric linker may be any combination of linkers consisting of SEQ. No.47 and glycine and serine residues, such as for example a fragment Gly Gly Gly Ser Gly/GGGSG or any fragment thereof acting as a steric linker, for example a fragment Gly Gly Gly/GGG. In such case the steric linker may be a combination of glycine, cysteine and alanine residues, such as for example Cys Ala Ala Cys Ala Ala Ala Cys Gly Gly Gly/CAACAAACGGG.

In other embodiment, the flexible steric linker may be a sequence Gly Gly Gly Cys Ala Ala Ala Cys Ala Ala Cys Gly Ser Gly/GGGCAAACAACGSG (SEQ. No.77) or any combination thereof.

In one embodiment, the flexible steric linker may be also selected from single amino acid residues, such as single cysteine residue.

Particular embodiment of the invention are fusion proteins selected from the group consisting of the proteins represented by SEQ. No. 1, SEQ. No. 4, SEQ. No. 5, and SEQ. No. 6 which comprise as the antiproliferative effector peptide the 34-amino acid fragment of human fetoprotein represented by SEQ. No. 27.

Other specific embodiment of the invention are fusion proteins selected from the group consisting of the proteins represented by SEQ. No. 2, SEQ. No. 3 and SEQ. No. 7 which comprise as the antiproliferative effector peptide the 8-amino acid fragment of human fetoprotein represented by SEQ. No. 28.

Other specific embodiment of the invention are fusion proteins selected from the group consisting of the proteins represented by SEQ. No. 8 and SEQ. No. 9, which comprise as the effector peptide the peptide derived from p21WAF represented by SEQ. No. 29.

Other specific embodiment of the invention is the fusion protein represented by SEQ. No. 10, which comprises as the effector peptide a 16-amino acid analogue of domain binding FGF-2 receptor represented by SEQ. No. 26.

Other specific embodiment of the invention is the fusion represented by SEQ. No. 11, which comprises as the effector peptide DD2 from DOC-2/DAB2 protein represented by SEQ. No. 30.

Other specific embodiment of the invention is the fusion protein represented by SEQ. No. 12, which comprises as the effector peptide an arginine deiminase from Mycoplasma arginini represented by SEQ. No. 31.

Other specific embodiment of the invention is the fusion protein represented by SEQ. No. 13, which comprises as the effector peptide a fragment of p16 peptide represented by SEQ. No. 32.

Other specific embodiment of the invention is the fusion protein represented by SEQ. No. 13, which comprises as the effector peptide a fragment of p16 peptide fused with a 17-amino-acid transporting domain of antennapedia represented by SEQ. No. 33.

Other specific embodiment of the invention is the fusion represented by SEQ. No. 14, which comprises as the effector peptide a fragment of MEK-1 protein represented by SEQ. No. 34.

Other specific embodiment of the invention is the fusion protein represented by SEQ. No. 15, which comprises as the effector peptide an N-terminal fragment of PH domain of TCL1 protein represented by SEQ. No. 35.

Other specific embodiment of the he invention is the fusion protein represented by SEQ. No. 16, which comprises as the effector peptide a hexapeptide Phe-Trp-Leu-Arg-Phe-Thr represented by SEQ. No. 36.

Other specific embodiment of the invention is the fusion protein represented by SEQ. No. 17, which comprises as the effector peptide a 13-amino acid fragment of tubulin represented by SEQ. No. 37.

Other specific embodiment of the invention is the fusion protein represented by SEQ. No. 18, which comprises as the effector peptide a 10-amino acid fragment of tubulin represented by SEQ. No. 39.

Other specific embodiment of the invention is the fusion protein represented by SEQ. No. 19, which comprises as the effector peptide melittin represented by SEQ. No. 39.

Other specific embodiment of the invention is the fusion protein represented by SEQ. No. 20, which comprises as the effector peptide a 6-amino acid peptide C2 based on sequence of bee defensin represented by SEQ. No. 40.

Other specific embodiment of the invention is the fusion protein represented by SEQ. No. 21, which comprises as the effector peptide the 8-amino acid peptide binding to FGF-2 ligand represented by SEQ. No. 41.

Other specific embodiment of the invention is the fusion protein represented by SEQ. No. 22, which comprises as the effector peptide the 15-amino acid peptide lasioglossin LL2 represented by SEQ. No. 42.

Other specific embodiment of the invention is the fusion protein represented by SEQ. No. 23, which comprises as the effector peptide the 13-amino acid peptide binding to SH3 domain of RasGAP represented by SEQ. No. 43.

Other specific embodiment of the invention is the fusion protein represented by SEQ. No. 25, which comprises as the effector peptide the analogue of Pep27 peptide represented by SEQ. No. 44.

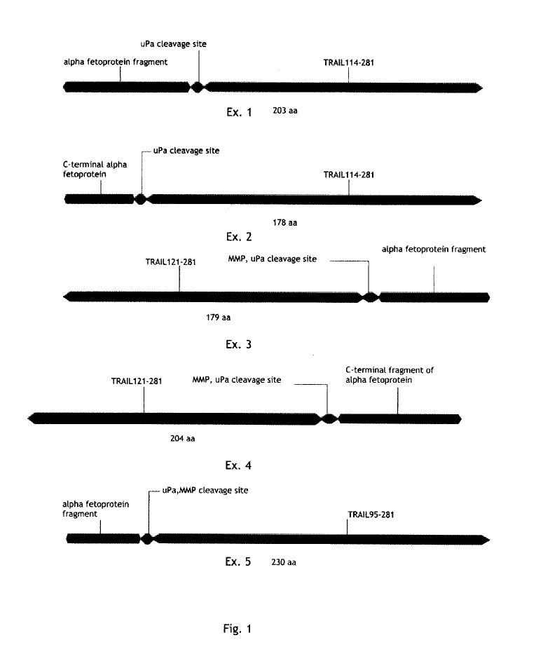

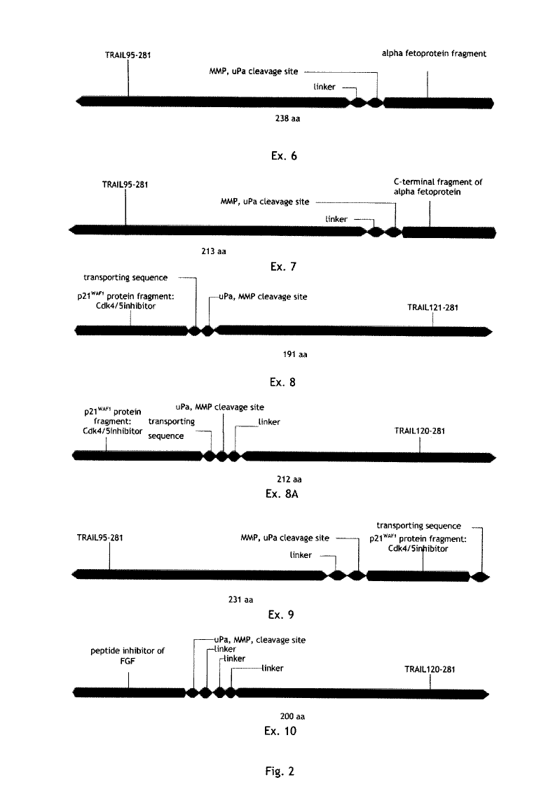

A detailed description of the structure of representative fusion proteins mentioned above are shown in FIGS. 1 to 5, and in the Examples presented below.

In accordance with the present invention, by the fusion protein it is meant a single protein molecule containing two or more proteins or fragments thereof, covalently linked via peptide bond within their respective peptide chains, without additional chemical linkers.

The fusion protein can also be alternatively described as a protein construct or a chimeric protein. According to the present invention, the terms “construct” or “chimeric protein”, if used, should be understood as referring to the fusion protein as defined above.

For a person skilled in the art it will be apparent that the fusion protein thus defined can be synthesized by known methods of chemical synthesis of peptides and proteins.

The fusion protein can be synthesized by methods of chemical peptide synthesis, especially using the techniques of peptide synthesis in solid phase using suitable resins as carriers. Such techniques are conventional and known in the art, and described inter alia in the monographs, such as for example Bodanszky and Bodanszky, The Practice of Peptide Synthesis, 1984, Springer-Verlag, New York, Stewart et al., Solid Phase Peptide Synthesis, 2nd Edition, 1984, Pierce Chemical Company.

The fusion protein can be synthesized by the methods of chemical synthesis of peptides as a continuous protein. Alternatively, the individual fragments (domains) of protein may be synthesized separately and then combined together in one continuous peptide via a peptide bond, by condensation of the amino terminus of one peptide fragment from the carboxyl terminus of the second peptide. Such techniques are conventional and well known.

For verification of the structure of the resulting peptide known methods of the analysis of amino acid composition of peptides may be used, such as high resolution mass spectrometry technique to determine the molecular weight of the peptide. To confirm the peptide sequence protein sequencers can also be used, which sequentially degrade the peptide and identify the sequence of amino acids.

Preferably, however, the fusion protein of the invention is a recombinant protein, generated by methods of gene expression of a polynucleotide sequence encoding the fusion protein in host cells.

A further aspect of the invention is the polynucleotide sequence, particularly DNA sequence encoding a fusion protein as defined above.

Preferably, the polynucleotide sequence, particularly DNA, according to the invention, encoding the fusion protein as defined above, is a sequence optimized for expression in E. coli.

Another aspect of the invention is also an expression vector containing the polynucleotide sequence, particularly DNA sequence of the invention as defined above.

Another aspect of the invention is also a host cell comprising an expression vector as defined above.

A preferred host cell for expression of fusion proteins of the invention is an E. coli cell.

Methods for generation of recombinant proteins, including fusion proteins, are well known. In brief, this technique consists in generation of polynucleotide molecule, for example DNA molecule encoding the amino acid sequence of the target protein and directing the expression of the target protein in the host. Then, the target protein encoding polynucleotide molecule is incorporated into an appropriate expression vector, which ensures an efficient expression of the polypeptide. Recombinant expression vector is then introduced into host cells for transfection/transformation, and as a result a transformed host cell is produced. This is followed by a culture of transformed cells to overexpress the target protein, purification of obtained proteins, and optionally cutting off by cleavage the tag sequences used for expression or purification of the protein.

Suitable techniques of expression and purification are described, for example in the monograph Goeddel, Gene Expression Technology, Methods in Enzymology 185, Academic Press, San Diego, Calif. (1990), and A. Staron et al., Advances Mikrobiol., 2008, 47, 2, 1983-1995.

Cosmids, plasmids or modified viruses can be used as expression vectors for the introduction and replication of DNA sequences in host cells. Typically plasmids are used as expression vectors. Suitable plasmids are well known and commercially available.

Expression vector of the invention comprises a polynucleotide molecule encoding the fusion protein of the invention and the necessary regulatory sequences for transcription and translation of the coding sequence incorporated into a suitable host cell. Selection of regulatory sequences is dependent on the type of host cells and can be easily carried out by a person skilled in the art. Examples of such regulatory sequences are transcriptional promoter and enhancer or RNA polymerase binding sequence, ribosome binding sequence, containing the transcription initiation signal, inserted before the coding sequence, and transcription terminator sequence, inserted after the coding sequence. Moreover, depending on the host cell and the vector used, other sequences may be introduced into the expression vector, such as the origin of replication, additional DNA restriction sites, enhancers, and sequences allowing induction of transcription.

The expression vector will also comprise a marker gene sequence, which confers defined phenotype to the transformed cell and enables specific selection of transformed cells. Furthermore, the vector may also contain a second marker sequence which allows to distinguish cells transformed with recombinant plasmid containing inserted coding sequence of the target protein from those which have taken up the plasmid without insert. Most often, typical antibiotic resistance markers are used, however, any other reporter genes known in the field may be used, whose presence in a cell (in vivo) can be easily determined using autoradiography techniques, spectrophotometry or bio- and chemi-luminescence. For example, depending on the host cell, reporter genes such as β-galactosidase. β-glucuronidase, luciferase, chloramphenicol acetyltransferase or green fluorescent protein may be used.

Furthermore, the expression vector may contain signal sequence, transporting proteins to the appropriate cellular compartment, e.g. periplasma, where folding is facilitated. Additionally a sequence encoding a label/tag, such as HisTag attached to the N-terminus or GST attached to the C-terminus, may be present, which facilitates subsequent purification of the protein produced using the principle of affinity, via affinity chromatography on a nickel column. Additional sequences that protect the protein against proteolytic degradation in the host cells, as well as sequences that increase its solubility may also be present.

Auxiliary element attached to the sequence of the target protein may block its activity, or be detrimental for another reason, such as for example due to toxicity. Such element must be removed, which may be accomplished by enzymatic or chemical cleavage. In particular, a six-histidine tag HisTag or other markers of this type attached to allow protein purification by affinity chromatography should be removed, because of its described effect on the liver toxicity of soluble TRAIL protein. Heterologous expression systems based on various well-known host cells may be used, including prokaryotic cells: bacterial, such as Escherichia coli or Bacillus subtilis, yeasts such as Saccharomyces cervisiae or Pichia pastoris, and eukaryotic cell lines (insect, mammalian, plant).

Preferably, due to the ease of culturing and genetic manipulation, and a large amount of obtained product, the E. coli expression system is used. Accordingly, the polynucleotide sequence containing the target sequence encoding the fusion protein of the invention will be optimized for expression in E. coli, i.e. it will contain in the coding sequence codons optimal for expression in E. coli, selected from the possible sequence variants known in the state of art. Furthermore, the expression vector will contain the above described elements suitable for E. coli attached to the coding sequence.

Accordingly, in a preferred embodiment of the invention a polynucleotide sequence comprising a sequence encoding a fusion protein of the invention, optimized for expression in E. coli is selected from the group of polynucleotide sequences consisting of:

SEQ. No. 50; SEQ. No. 51; SEQ. No. 52, SEQ. No. 53; SEQ. No. 54; SEQ. No. 55; SEQ. No. 56; SEQ. No. 57; SEQ. No. 58; SEQ. No. 59; SEQ. No. 60, and SEQ. No. 61; SEQ, No. 62; SEQ. No. 63; SEQ. No. 64; SEQ. No. 65; SEQ. No. 66, SEQ. No. 67; SEQ, No. 68; SEQ. No 69; SEQ. No. 70; SEQ. No. 71; SEQ. No. 72; SEQ. No. 73; SEQ. No. 74 and SEQ. No. 76.

which encode a fusion protein having an amino acid sequence corresponding to amino acid sequences selected from the group consisting of amino acid sequences, respectively:

SEQ. No. 1; SEQ. No. 2; SEQ. No. 3; SEQ. No. 4; SEQ. No. 5; SEQ. No. 6; SEQ. No. 7; SEQ. No. 8; SEQ. No. 9; SEQ. No. 10; SEQ. No. 11; SEQ. No. 12; SEQ. No. 13; SEQ. No. 14; SEQ. No. 15; SEQ. No. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25 and SEQ. No. 75.

In a preferred embodiment, the invention provides also an expression vector suitable for transformation of E. coli, comprising the polynucleotide sequence selected from the group of polynucleotide sequences SEQ. No. 50 to SEQ. No. 74 and SEQ. No. 76 indicated above, as well as E. coli cell transformed with such an expression vector.

Transformation, i.e. introduction of a DNA sequence into bacterial host cells, particularly E. coil, is usually performed on the competent cells, prepared to take up the DNA for example by treatment with calcium ions at low temperature (4′C.), and then subjecting to the heat-shock (at 37-42° C.) or by electroporation. Such techniques are well known and are usually determined by the manufacturer of the expression system or are described in the literature and manuals for laboratory work, such as Maniatis et al., Molecular Cloning. Cold Spring Harbor, N.Y., 1982).

The procedure of overexpression of fusion proteins of the invention in E. coli expression system will be further described below.

The invention also provides a pharmaceutical composition containing the fusion protein of the invention as defined above as an active ingredient and a suitable pharmaceutically acceptable carrier, diluent and conventional auxiliary components. The pharmaceutical composition will contain an effective amount of the fusion protein of the invention and pharmaceutically acceptable auxiliary components dissolved or dispersed in a carrier or diluent, and preferably will be in the form of a pharmaceutical composition formulated in a unit dosage form or formulation containing a plurality of doses. Pharmaceutical forms and methods of their formulation as well as other components, carriers and diluents are known to the skilled person and described in the literature. For example, they are described in the monograph Remington's Pharmaceutical Sciences, ed. 20, 2000, Mack Publishing Company, Easton, USA.

The terms “pharmaceutically acceptable carrier, diluent, and auxiliary ingredient” comprise any solvents, dispersion media, surfactants, antioxidants, stabilizers, preservatives (e.g. antibacterial agents, antifungal agents), isotonizing agents, known in the art. The pharmaceutical composition of the s invention may contain various types of carriers, diluents and excipients, depending on the chosen route of administration and desired dosage form, such as liquid, solid and aerosol forms for oral, parenteral, inhaled, topical, and whether that selected form must be sterile for administration route such as by injection. The preferred route of administration of the pharmaceutical composition according to the invention is parenteral, including injection routes such as intravenous, intramuscular, subcutaneous, intraperitoneal, intratumoral, or by single or continuous intravenous infusions.

In one embodiment, the pharmaceutical composition of the invention may be administered by injection directly to the tumor. In another embodiment, the pharmaceutical composition of the invention may be administered intravenously. In yet another embodiment, the pharmaceutical composition of the invention can be administered subcutaneously or intraperitoneally. A pharmaceutical composition for parenteral administration may be a solution or dispersion in a pharmaceutically acceptable aqueous or non-aqueous medium, buffered to an appropriate pH and isoosmotic with body fluids, if necessary, and may also contain antioxidants, buffers, bacteriostatic agents and soluble substances, which make the composition compatible with the tissues or blood of recipient. Other components, which may included in the composition, are for example water, alcohols such as ethanol, polyols such as glycerol, propylene glycol, liquid polyethylene glycol, lipids such as triglycerides, vegetable oils, liposomes. Proper fluidity and the particles size of the substance may be provided by coating substances, such as lecithin, and surfactants, such as hydroxypropyl celulose polysorbates, and the like.

Suitable isotonizing agents for liquid parenteral compositions are, for example, sugars such as glucose, and sodium chloride, and combinations thereof.

Alternatively, the pharmaceutical composition for administration by injection or infusion may be in a powder form, such as a lyophilized powder for reconstitution immediately prior to use in a suitable carrier such as, for example, sterile pyrogen-free water.

The pharmaceutical composition of the invention for parenteral administration may also have the form of nasal administration, including solutions, sprays or aerosols. Preferably, the form for intranasal administration will be an aqueous solution and will be isotonic or buffered o maintain the pH from about 5.5 to about 6.5, so as to maintain a character similar to nasal secretions. Moreover, it will contain preservatives or stabilizers, such as in the well-known intranasal preparations.

The composition may contain various antioxidants which delay oxidation of one or more components. Furthermore, in order to prevent the action of microorganisms, the composition may contain various antibacterial and anti fungal agents, including, for example, and not limited to, parabens, chlorobutanol, himerosal, sorbic acid, and similar known substances of this type. In general, the pharmaceutical composition of the invention can include, for example at least about 0.01 wt % of active ingredient. More particularly, the composition may contain the active ingredient in the amount from 1% to 75% by weight of the composition unit, or for example from 25% to 60% by weight, but not limited to the indicated values. The actual amount of the dose of the composition according to the present invention administered to patients, including man, will be determined by physical and physiological factors, such as body weight, severity of the condition, type of disease being treated, previous or concomitant therapeutic interventions, the patient and the route of administration. A suitable unit dose, the total dose and the concentration of active ingredient in the composition is to be determined by the treating physician.

The composition may for example be administered at a dose of about 1 microgram/kg of body weight to about 1000 mg/kg of body weight of the patient, for example in the range of 5 mg/kg of body weight to 100 mg/kg of body weight or in the range of 5 mg/kg of body weight to 500 mg/kg of body weight. The fusion protein and the compositions containing it exhibit anticancer or antitumor and can be used for the treatment of cancer diseases. The invention also provides the use of the fusion protein of the invention as defined above for treating cancer diseases in mammals, including humans. The invention also provides a method of treating neoplastic/cancer diseases in mammals, including humans, comprising administering to a subject in need of such treatment an anit-neoplasticc/anticancer effective amount of the fusion protein of the invention as defined above, optionally in the form of appropriate pharmaceutical composition.

The fusion protein of the invention can be used for the treatment of hematologic malignancies, such as leukaemia, granulomatosis, myeloma and other hematologic malignancies. The fusion protein can also be used for the treatment of solid tumors, such as breast cancer, lung cancer, including non-small cell lung cancer, colon cancer, pancreatic cancer, ovarian cancer, bladder cancer, prostate cancer, kidney cancer, brain cancer, and the like. Appropriate route of administration of the fusion protein in the treatment of cancer will be in particular parenteral route, which consists in administering the fusion protein of the invention in the form of injections or infusions, in the composition and form appropriate for this administration route. The invention will be described in more detail in the following general procedures and examples of specific fusion proteins.

General Procedure for Overexpression of the Fusion Protein

Preparation of a Plasmid

Amino acid sequence of the target fusion protein was used as a template to 3generate a DNA sequence encoding it, comprising codons optimized for expression in Escherichia coli. Such a procedure allows to increase the efficiency of a further step of target protein synthesis in Escherichia coli. Resulting nucleotide sequence was then automatically synthesized. Additionally, the cleavage sites of restriction enzymes NdeI (at the 5′-end of leading strand) and XhoI (at the 3′-end of leading strand) were added to the resulting gene encoding the target protein. These were used to clone the gene into the vector pET28a (Novagen). They may be also be used for cloning the gene encoding the protein to other vectors. Target protein expressed from this construct can be optionally equipped at the N-terminus with a polyhistidine tag (six histidines), preceded by a site recognized by thrombin, which subsequently served to its purification via affinity chromatography. Some target were expressed without any tag, in particular without histidine tag, and those were subsequently purified on SP Sepharose. The correctness of the resulting construct was confirmed firstly by restriction analysis of isolated plasmids using the enzymes NdeI and XhoI, followed by automatic sequencing of the entire reading frame of the target protein. The primers used for sequencing were complementary to the sequences of T7 promoter (5′-TAATACGACTCACTATAGG-3′) and 17 terminator (5-GCTAGTTATTGCTCAGCGG-3′) present in the vector. Resulting plasmid was used for overexpression of the target fusion protein in a commercial E. coli strain, which was transformed according to the manufacturers recommendations. Colonies obtained on the selection medium (LB agar, kanamycin 50 μg/ml, 1% glucose) were used for preparing an overnight culture in LB liquid medium supplemented with kanamycin (50 μg/ml) and 1% glucose. After about 15 h of growth in shaking incubator, the cultures were used to inoculate the appropriate culture.

Overexpression and Purification of Fusion Proteins—General Procedure A

LB medium with kanamycin (30 μg/ml) and 100 μM zinc sulfate was inoculated with overnight culture. The culture was incubated at 37° C. until the optical density (OD) at 600 nm reached 0.60-0.80. Then IPTG was added to the final concentration in the range of 0.25-1 mM. After incubation (3.5-20 h) with shaking at 25° C. the culture was centrifuged for 25 min at 6,000 g. Bacterial pellets were resuspended in a buffer containing 50 mM KH₂PO₄, 0.5 M NaCl, 10 mM imidazole, pH 7.4. The suspension was sonicated on ice for 8 minutes (40% amplitude, 15-second pulse, 10 s interval). The resulting extract was clarified by centrifugation for 40 minutes at 20000 g, 4° C. Ni-Sepharose (GE Healthcare) resin was pre-treated by equilibration with buffer, which was used for preparation of the bacterial cells extract. The resin was then incubated overnight at 4° C. with the supernatant obtained after centrifugation of the extract. Then it was loaded into chromatography column and washed with 15 to 50 volumes of buffer 50 mM KH₂PO₄, 0.5 M NaCl, 20 mM imidazole, pH 7.4. The obtained protein was eluted from the column using imidazole gradient in 50 mM KH₂PO₄ buffer with 0.5 M NaCl, pH 7.4. Obtained fractions were analyzed by SDS-PAGE. Appropriate fractions were combined and dialyzed overnight at 4° C. against 50 mM Tris buffer, pH 7.2, 150 mM NaCl, 500 mM L-arginine, 0.1 mM ZnSO₄, 0.01% Tween 20, and at the same time Histag, if present, was cleaved with thrombin (1:50). After the cleavage, thrombin was separated from the target fusion protein expressed with His tag by purification using Benzamidine Sepharose™ resin. Purification of target fusion proteins expressed without Histag was performed on SP Sepharose. The purity of the product was analyzed by SDS-PAGE electrophoresis (Maniatis et al, Molecular Cloning. Cold Spring Harbor, N.Y., 1982).

Overexpression and Purification of Fusion Proteins—General Procedure B

LB medium with kanamycin (30 μg/ml) and 100 μM zinc sulfate was inoculated with overnight culture. Cultures were incubated at 37° C. until optical density (OD) at 600 nm reached 0.60-0.80. Then IPTG was added to the final is concentration in the range 0.5-1 mM. After 20 h incubation with shaking at 25° C. the culture was centrifuged for 25 min at 6000 g. Bacterial cells after overexpression were disrupted in a French Press in a buffer containing 50 mM KH₂PO₄, 0.5 M NaCl, 10 mM imidazole, 5 mM beta-mercaptoethanol, 0.5 mM PMSF (phenylmethylsulphonyl fluoride), pH 7.8. Resulting extract was clarified by centrifugation for 50 minutes at 8000 g. The Ni-Sepharose resin was incubated overnight with the obtained supernatant. Then the resin with bound protein was packed into the chromatography column. To wash-out the fractions containing non-binding proteins, the column was washed with 15 to 50 volumes of buffer 50 mM KH2PO₄, 0.5 M NaCl, 10 mM imidazole, 5 mM beta-mercaptoethanol, 0.5 mM PMSF (phenylmethylsulphonyl fluoride), pH 7.8. Then, to wash-out the majority of proteins binding specifically with the bed, the column was washed with a buffer containing 50 mM KH2PO4, 0.5 M NaCl, 500 mM imidazole, 10% glycerol, 0.5 mM PMSF, pH 7.5. Obtained fractions were analyzed by SDS-PAGE (Maniatis et al, Molecular Cloning. Cold Spring Harbor, N.Y., 1982). The fractions containing the target protein were combined and, if the protein was expressed with histidine tag, cleaved with thrombin (1 U per 4 mg of protein, 8 h at 16° C.) to remove polyhistidine tag. Then the fractions were dialyzed against formulation buffer (500 mM L-arginine, 50 mM Tris, 2.5 mM ZnSO₄, pH 7.4).

Further in this description proteins originally expressed with histidine tag that was subsequently removed are designated as a) at the Ex. No. Proteins that were originally expressed without histidine tag are designated as b) at the Ex. No.

### EXAMPLE 1

### The Fusion Protein of SEQ. No. 1

The protein of SEQ. No. 1 is a fusion protein having the length of 203 amino acids and the mass of 23.3 kDa, in which at the N-terminus of the sequence TRAIL114-281 a 34-amino acid fragment of human fetoprotein (SEQ. No. 27) is attached as an effector peptide. Between the effector peptide and the sequence of TRAIL there is incorporated a sequence of cleavage site recognized by urokinase uPA (SEQ. No. 46) due to which the effector peptide will undergo cleavage in the tumor environment.

Structure of the fusion protein is shown schematically in FIG. 1 and its amino acid sequence and the DNA encoding sequence comprising codons optimized for expression in E. coli are, respectively, SEQ. No. 1 and SEQ. No. 50 as shown in the attached Sequence Listing.

The amino acid sequence SEQ. No. 1 of the structure described above was used as a template to generate its coding DNA sequence SEQ. No. 50. A plasmid containing the coding sequence of DNA was generated and overexpression of the fusion protein was carried out in accordance with the general procedures described above. Overexpression was performed according to the general procedure A, using E. coli BL21 (DE3) or Tuner(DE3)pLysS strains from Novagen. The protein was separated by electrophoresis in accordance with the general procedure described above.

### EXAMPLE 2

### The Fusion Protein of SEQ. No. 2

The protein of SEQ. No. 2 is a fusion protein having the length of 178 amino acids and the mass of 20.5 kDa, in which at the N-terminus of the sequence TRAIL114-281 a 8-amino acid fragment of human fetoprotein (SEQ. No. 28) is attached as an effector peptide. Between the effector peptide and the sequence of TRAIL there is incorporated a sequence of cleavage site recognized by urokinase uPA (SEQ. No. 46) due to which the effector peptide will undergo cleavage in the tumor environment.

Structure of the fusion protein is shown schematically in FIG. 1 and its amino acid sequence and the DNA encoding sequence comprising codons optimized for expression in E. coli are, respectively, SEQ. No. 2 and SEQ. No. 51 as shown in the attached Sequence Listing.

The amino acid sequence SEQ. No. 2 of the structure described above was used as a template to generate its coding DNA sequence SEQ. No. 51. A plasmid containing the coding sequence of DNA was generated and overexpression of the fusion protein was carried out in accordance with the general procedures described above. Overexpression was performed according to the general procedure B, using E. coli BL21 (DE3) strain from Novagen. The protein was separated by electrophoresis in accordance with the general procedure is described above.

### EXAMPLE 3

### The Fusion Protein of SEQ. No. 3

The protein of SEQ. No. 3 is a fusion protein having the length of 179 amino acids and the mass of 20.5 kDa, in which at the C-terminus of the sequence TRAIL121-281 a 8-amino acid fragment of human fetoprotein (SEQ. No. 28) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment.

Structure of the fusion protein is shown schematically in FIG. 1 and its amino acid sequence and the DNA encoding sequence comprising codons optimized for expression in E. coil are, respectively, SEQ. No. 3 and SEQ. No. 52 as shown in the attached Sequence Listing.

The amino acid sequence SEQ. No. 3 of the structure described above was used as a template to generate its coding DNA sequence SEQ. No. 52. A plasmid containing the coding sequence of DNA was generated and overexpression of the fusion protein was carried out in accordance with the general procedures described above. Overexpression was performed according to the general procedure A, using E. coli BL21 (DE3) strain from Novagen. The protein was separated by electrophoresis in accordance with the general procedure described above.

### EXAMPLE 4

### The Fusion Protein of SEQ. No. 4

The protein of SEQ. No. 4 is a fusion protein having the length of 204 amino acids and the mass of 23.2 kDa, in which at the C-terminus of the sequence TRAIL121-281 a 34-amino acid fragment of human fetoprotein (SEQ. No. 27) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment.

Structure of the fusion protein is shown schematically in FIG. 1 and its amino acid sequence and the DNA encoding sequence comprising codons optimized for expression in E. coil are, respectively, SEQ. No. 4 and SEQ. No. 53 as shown in the attached Sequence Listing.

The amino acid sequence SEQ. No. 4 of the structure described above was used as a template to generate its coding DNA sequence SEQ. No. 53. A plasmid containing the coding sequence of DNA was generated and overexpression of the fusion protein was carried out in accordance with the general procedures described above. Overexpression was performed according to the general procedure B, using E. coli BL21DE3pLysSRIL strain from Stratagene or Tuner (DE3) strain from Novagen. The protein was separated by electrophoresis in accordance with the general procedure described above.

### EXAMPLE 5

### The Fusion Protein of SEQ. No. 5

The protein of SEQ. No. 5 is a fusion protein having the length of 230 amino acids and the mass of 26 kDa, in which at the N-terminus of the sequence TRAIL95-281 a 34-amino acid fragment of human fetoprotein (SEQ. No. 27) is attached as an effector peptide. Between the effector peptide and the sequence of TRAIL there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment.

Structure of the fusion protein is shown schematically in FIG. 1 and its amino acid sequence and the DNA encoding sequence comprising codons optimized for expression in E. coil are, respectively, SEQ. No. 5 and SEQ. No. 54 as shown in the attached Sequence Listing.

The amino acid sequence SEQ. No. 5 of the structure described above was used as a template to generate its coding DNA sequence SEQ. No. 54. A plasmid containing the coding sequence of DNA was generated and overexpression of the fusion protein was carried out in accordance with the general procedures described above. Overexpression was performed according to the general procedure A, using E. coil Tuner (DE3) strain from Novagen. The protein was separated by electrophoresis in accordance with the general procedure described above.

### EXAMPLE 6

### The Fusion Protein of SEQ. No. 6

The protein of SEQ. No. 6 is a fusion protein having the length of 238 amino acids and the mass of 26.7 kDa, in which at the C-terminus of the sequence TRAIL95-281 a 34-amino acid fragment of human fetoprotein (SEQ. No. 27) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. Between the sequence of TRAIL and the sequence of cleavage site recognized by metalloprotease MMP the fusion protein contains additionally a flexible cysteine—alanine linker (SEQ. No. 47).

Structure of the fusion protein is shown schematically in FIG. 2 and its amino acid sequence and the DNA encoding sequence comprising codons optimized for expression in E. coli are, respectively, SEQ. No. 6 and SEQ. No. 55 as shown in the attached Sequence Listing.

The amino acid sequence SEQ. No. 6 of the structure described above was used as a template to generate its coding DNA sequence SEQ. No. 55. A plasmid containing the coding sequence of DNA was generated and overexpression of the fusion protein was carried out in accordance with the general procedures described above. Overexpression was performed according to the general procedure A, using E. coil Tuner (DE3) strain from Novagen. The protein was separated by electrophoresis in accordance with the general procedure described above.

### EXAMPLE 7

### The Fusion Protein of SEQ. No. 7

The protein of SEQ. No. 7 is a fusion protein having the length of 213 amino acids and the mass of 24.1 kDa, in which at the C-terminus of the sequence TRAIL95-281 a 8-amino acid fragment of human fetoprotein (SEQ. No. 28) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. Between the sequence of TRAIL and the sequence of cleavage site recognized by metalloprotease MMP the fusion protein contains additionally a flexible cysteine—alanine linker (SEQ. No. 47).

Structure of the fusion protein is shown schematically in FIG. 2 and its amino acid sequence and the DNA encoding sequence comprising codons optimized for expression in E. coil are, respectively, SEQ. No. 7 and SEQ. No. 56 as shown in the attached Sequence Listing.

The amino acid sequence SEQ. No. 7 of the structure described above was used as a template to generate its coding DNA sequence SEQ. No. 56. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

### EXAMPLE 8

### The Fusion Protein of SEQ. No. 8

The protein of SEQ. No. 8 is a fusion protein having the length of 191 amino acids and the mass of 23 kDa, in which at the N-terminus of the sequence TRAIL121-281 a 20-amino acid fragment of peptide derived from p21WAF protein (SEQ. No. 29) is attached as an effector peptide. Additionally, at the C-terminus of the effector protein there is attached a fragment of antennapedia protein domain (SEQ. No. 49) as a transporting sequence, which aids in penetration of the cell membrane and transportation of the fusion protein into the cell. Between the transporting sequence and the sequence of TRAIL there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment.

Structure of the fusion protein is shown schematically in FIG. 2 and its amino acid sequence and the DNA encoding sequence comprising codons optimized for expression in E. coli are, respectively, SEQ. No. 8 and SEQ. No. 57 as shown in the attached Sequence Listing.

The amino acid sequence SEQ. No. 8 of the structure described above was used as a template to generate its coding DNA sequence SEQ. No. 57. A plasmid containing the coding sequence of DNA in two versions, one allowing to express His tag and a site recognized by thrombin and the second without any tag was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

### EXAMPLE 8A

### The Fusion Protein of SEQ. No. 75

The protein of SEQ. No. 75 is a fusion protein having the length of 212 amino acids and the mass of 24,13 kDa, in which at the N-terminus of the sequence TRAIL120-281 a 20-amino acid fragment of peptide derived from p21WAF protein (SEQ. No. 29) is attached as an effector peptide. Additionally, at the C-terminus of the effector protein there is attached a fragment of antennapedia protein domain (SEQ. No. 49) as a transporting sequence, which aids in penetration of the cell membrane and transportation of the fusion protein into the cell. Between the transporting sequence and the sequence of TRAIL there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally between the metalloprotease cleavage site and the sequence of TRAIL the fusion protein contains additionally a flexible linker (SEQ. No. 77).

Structure of the fusion protein is shown schematically in FIG. 2 and its amino acid sequence and the DNA encoding sequence comprising codons optimized for expression in E. coli are, respectively, SEQ. No. 75 and SEQ. No. 76 as shown in the attached Sequence Listing.

The amino acid sequence SEQ. No. 75 of the structure described above was used as a template to generate its coding DNA sequence SEQ. No. 76. A plasmid containing the coding sequence of DNA in two versions, one allowing to express His tag and a site recognized by thrombin and the second without any tag was generated and overexpression of the fusion protein was carried out in accordance with the general procedures described above. Overexpression was performed according to the general procedure A, using E. coil Tuner (DE3) strain from Novagen. The protein was separated by electrophoresis in accordance with the general procedure described above.

### EXAMPLE 9

### The Fusion Protein of SEQ. No. 9

The protein of SEQ. No. 9 is a fusion protein having the length of 231 amino acids and the mass of 26.5 kDa, in which at the C-terminus of the sequence TRAIL95-281 a 20-amino acid fragment of peptide derived from p21WAF protein (SEQ. No. 29) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. Between the sequence of TRAIL and the sequence of cleavage sites the fusion protein contains additionally a flexible cysteine—alanine linker (SEQ. No. 47). Additionally, at the C-terminus of the effector protein there is attached a fragment of antennapedia protein domain (SEQ. No. 49) forming C-terminal fragment of entire construct as a transporting sequence which aids in penetration of the cell membrane and transportation of the fusion protein into the cell.

Structure of the fusion protein is shown schematically in FIG. 2 and its amino acid sequence and the DNA encoding sequence comprising codons optimized for expression in E. coil are, respectively, SEQ. No. 9 and SEQ. No. 58 as shown in the attached Sequence Listing.

The amino acid sequence SEQ. No. 9 of the structure described above was used as a template to generate its coding DNA sequence SEQ. No. 58. A plasmid containing the coding sequence of DNA was generated and overexpression of the fusion protein was carried out in accordance with the general procedures is described above. Overexpression was performed according to the general procedure A, using E. coli Rosetta (DE3) strain from Novagen. The protein was separated by electrophoresis in accordance with the general procedure described above.

### EXAMPLE 10

### The Fusion Protein of SEQ. No. 10

The protein of SEQ. No. 10 is a fusion protein having the length of 200 amino acids and the mass of 22.8 kDa, in which at the N-terminus of the sequence TRAIL120-281 a 16-amino acid fragment of peptide analogue of domain binding to FGF-2 receptor (SEQ. No. 26) is attached as an effector peptide. Between the effector peptide and the sequence of TRAIL there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Between the sequence of TRAIL and the sequence of cleavage sites the fusion protein contains additionally a flexible cysteine—alanine linker (SEQ. No. 47). Additionally, between the sequence of cleavage site and the sequence of flexible linker as well as between the sequence of flexible linker and TRAIL domain there is incorporated a linker consisting of two glycine residues aids in stabilization of trimeric structure.

Structure of the fusion protein is shown schematically in FIG. 2 and its amino acid sequence and the DNA encoding sequence comprising codons optimized for expression in E. coli are, respectively, SEQ. No. 10 and SEQ. No. 59 as shown in the attached Sequence Listing.

The amino acid sequence SEQ. No. 10 of the structure described above was used as a template to generate its coding DNA sequence SEQ. No. 59. A plasmid containing the coding sequence of DNA was generated and overexpression of the fusion protein was carried out in accordance with the general procedures described above. Overexpression was performed according to the general procedure A, using E. coli BL21 (DE3) strain from Novagen. The protein was separated by electrophoresis in accordance with the general procedure described above.

### EXAMPLE 11

### The Fusion Protein of SEQ. No. 11

The protein of SEQ. No. 11 is a fusion protein having the length of 233 amino acids and the mass of 26.5 kDa, in which at the C-terminus of the sequence TRAIL95-281 an 18-amino acid fragment of peptide DD2 derived from DOC-2/DAB2 (SEQ. No. 30) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. The sequence of the effector peptide has attached at its N-terminus the poly-arginine transporting domain consisting of 7 Arg residues. Transporting sequence aids in penetration of the cell membrane and transportation of the fusion protein into the cell. Between the sequence of TRAIL and the sequence of cleavage sites the fusion protein contains additionally a flexible cysteine—alanine—glycine linker CAACAAACGGG.

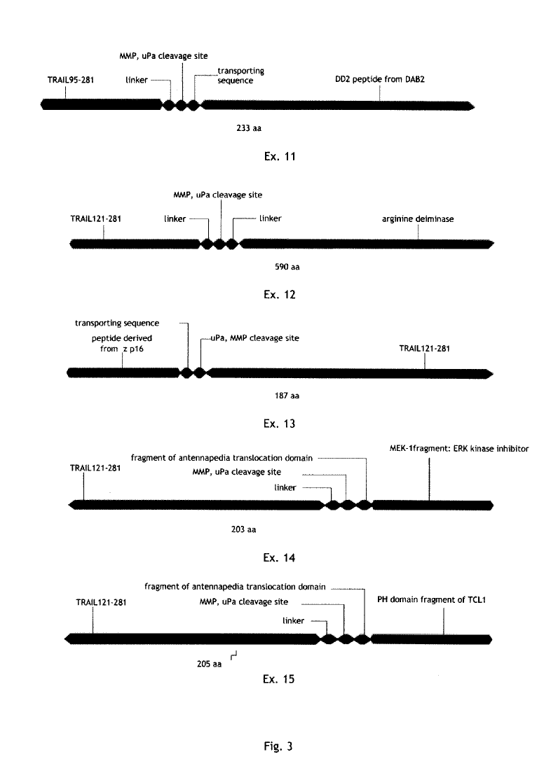

Structure of the fusion protein is shown schematically in FIG. 3 and its amino acid sequence and the DNA encoding sequence comprising codons optimized for expression in E. coil are, respectively, SEQ. No. 11 and SEQ. No. 60 as shown in the attached Sequence Listing.

The amino acid sequence SEQ. No. 11 of the structure described above was used as a template to generate its coding DNA sequence SEQ. No. 60. A plasmid containing the coding sequence of DNA was generated and overexpression of the fusion protein was carried out in accordance with the general procedures described above. Overexpression was performed according to the general procedure A, using E. coli BL21 (DE3) or Tuner(DE3)pLysS strains from Novagen. The protein was separated by electrophoresis in accordance with the general procedure described above.

### EXAMPLE 12

### The Fusion Protein of SEQ. No. 12

The protein of SEQ. No. 12 is a fusion protein having the length of 590 amino acids and the mass of 66.7 kDa, in which at the C-terminus of the sequence TRAIL121-281 an arginine deiminase from Mycoplasma arginini (SEQ. No. 31) is attached as an effector peptide. Between the effector peptide and the sequence of TRAIL there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. Between the sequence of TRAIL and the sequence of metalloprotease cleavage site the fusion protein contains additionally a flexible linker consisting of glycine and serine residues Gly Gly Ser Gly. Between the sequence of urokinase cleavage site and the sequence of effector protein the fusion protein contains additionally a flexible glycine serine linker Gly Gly Gly Ser Gly.

Structure of the fusion protein is shown schematically in FIG. 3 and its amino acid sequence and the DNA encoding sequence comprising codons optimized for expression in E. coli are, respectively, SEQ. No. 12 and SEQ. No. 61 as shown in the attached Sequence Listing.

The amino acid sequence SEQ. No. 12 of the structure described above was used as a template to generate its coding DNA sequence SEQ. No. 61. A plasmid containing the coding sequence of DNA was generated and overexpression of the fusion protein was carried out in accordance with the general procedures described above. Overexpression was performed according to the general procedure A, using E. coil BL21 (DE3) strain from Novagen. The protein was separated by electrophoresis in accordance with the general procedure described above.

### EXAMPLE 13

### The Fusion Protein of SEQ. No. 13

The protein of SEQ. No. 13 is a fusion protein having the length of 187 amino acids and the mass of 21.6 kDa, in which at the N-terminus of the sequence TRAIL121-281 a 10-amino acid peptide from p16 protein (SEQ. No. 32) is attached as an effector peptide. Between the effector peptide and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. The sequence of the effector peptide has attached at its C-terminus a transporting sequence (SEQ. No. 49) consisting of fragment of antennapedia protein domain fragment. Transporting sequence aids in penetration of the cell membrane and transportation of the fusion protein into the cell.

Structure of the fusion protein is shown schematically in FIG. 3 and its amino acid sequence and the DNA encoding sequence comprising codons optimized for expression in E. coil are, respectively, SEQ. No. 13 and SEQ. No. 62 as shown in the attached Sequence Listing.

The amino acid sequence SEQ. No. 13 of the structure described above was used as a template to generate its coding DNA sequence SEQ. No. 62. A plasmid containing the coding sequence of DNA was generated and overexpression of the fusion protein was carried out in accordance with the general procedures described above. Overexpression was performed according to the general procedure B, using E. coli BL21 (DE3) strain from Novagen or BL21DE3pLysSRIL strain from Stratagene. The protein was separated by electrophoresis in accordance with the general procedure described above.

### EXAMPLE 14

### The Fusion Protein of SEQ. No. 14

The protein of SEQ. No. 14 is a fusion protein having the length of 203 amino acids and the mass of 23.6 kDa, in which at the C-terminus of the sequence TRAIL121-281 a 13-amino acid fragment of MEK-1 protein—an inhibitor of ERK activation (SEQ. No. 34) is attached as an effector peptide. Between the C-terminus of TRAIL and the effector peptide domain there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. The sequence of the effector peptide has attached at its N-terminus a transporting sequence (SEQ. No. 48) consisting of antennapedia protein domain fragment. Transporting sequence aids in penetration of the cell membrane and transportation of the fusion protein into the cell. Between the sequence of TRAIL and the sequence of cleavage sites the fusion protein contains additionally a flexible glycine -cysteine linker GS.

Structure of the fusion protein is shown schematically in FIG. 3 and its amino acid sequence and the DNA encoding sequence comprising codons optimized for expression in E. coli are, respectively, SEQ. No. 14 and SEQ. No. 63 as shown in the attached Sequence Listing.

The amino acid sequence SEQ. No. 14 of the structure described above was used as a template to generate its coding DNA sequence SEQ. No. 63. A plasmid containing the coding sequence of DNA was generated and overexpression of the fusion protein was carried out in accordance with the general procedures described above. Overexpression was performed according to the general procedure B, using E. coil B.21 (DE3) strain from Novagen or BL21DE3pLysSRIL strain from Stratagene. The protein was separated by electrophoresis in accordance with the general procedure described above.

### EXAMPLE 15

### The Fusion Protein of SEQ. No. 15

The protein of SEQ. No. 15 is a fusion protein having the length of 205 amino acids and the mass of 24 kDa, in which at the C-terminus of the sequence TRAIL121-281 a 15-amino acid N-terminal fragment of PH domain of TCL1 protein—acting as Akt coactivator (SEQ. No. 35) is attached as an effector peptide. Between the TRAIL domain and the effector peptide there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. The sequence of the effector peptide has attached at its N-terminus a transporting sequence (SEQ. No. 48) consisting of fragment of antennapedia protein domain fragment. Transporting sequence aids in penetration of the cell membrane and transportation of the fusion protein into the cell. Between the sequence of TRAIL and the sequence of cleavage sites the fusion protein contains additionally a flexible glycine -cysteine linker GS.

Structure of the fusion protein is shown schematically in FIG. 3 and its amino acid sequence and the DNA encoding sequence comprising codons optimized for expression in E. coil are, respectively, SEQ. No. 15 and SEQ. No. 64 as shown in the attached Sequence Listing.

The amino acid sequence SEQ. No. 15 of the structure described above was used as a template to generate its coding DNA sequence SEQ. No. 64. A plasmid containing the coding sequence of DNA was generated and overexpression of the fusion protein was carried out in accordance with the general procedures described above. Overexpression was performed according to the general procedure B, using E. coil B.21 (DE3) or Tuner (DE3) strains from Novagen. The protein was separated by electrophoresis in accordance with the general procedure described above.

### EXAMPLE 16

### The Fusion Protein of SEQ. No. 16

The protein of SEQ. No. 16 is a fusion protein having the length of 183 amino acids and the mass of 21.2 kDa, in which at the N-terminus of the sequence TRAIL121-281 a hexapeptide acting as inhibitor of E2F (SEQ. No. 36) is attached as an effector peptide. Between the effector peptide and the TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, the sequence of the effector peptide has attached at its C-terminus a transporting sequence (SEQ. No. 49) consisting of fragment of antennapedia protein domain fragment. Transporting sequence aids in penetration of the cell membrane and transportation of the fusion protein into the cell.

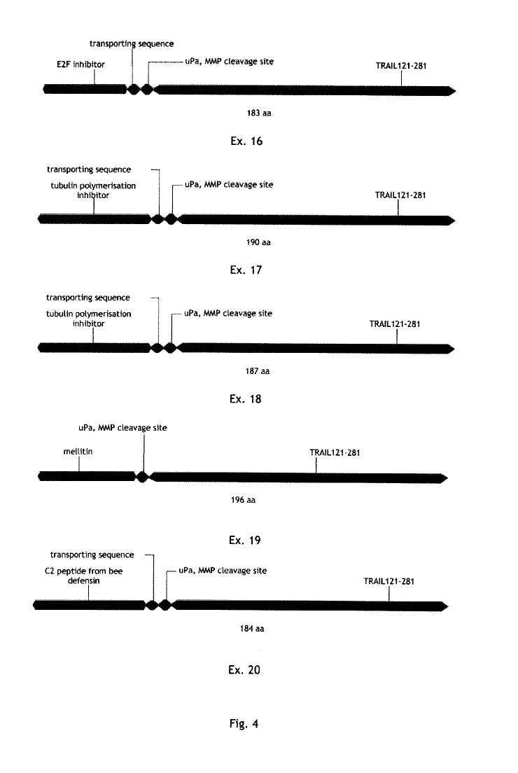

Structure of the fusion protein is shown schematically in FIG. 4 and its amino acid sequence and the DNA encoding sequence comprising codons optimized for expression in E. coli are, respectively, SEQ. No. 16 and SEQ. No. 65 as shown in the attached Sequence Listing.

The amino acid sequence SEQ. No. 16 of the structure described above was used as a template to generate its coding DNA sequence SEQ. No. 65. A plasmid containing the coding sequence of DNA was generated and overexpression of the fusion protein was carried out in accordance with the general procedures described above. Overexpression was performed according to the general procedure B, using E. coil B.21 (DE3) or Tuner (DE3) strains from Novagen. The protein was separated by electrophoresis in accordance with the general procedure described above.

### EXAMPLE 17

### The Fusion Protein of SEQ. No. 17

The protein of SEQ. No. 17 is a fusion protein having the length of 190 amino acids and the mass of 22.3 kDa, in which at the N-terminus of the sequence TRAIL121-281 a 13-amino acid fragment of tubulin (SEQ. No. 37) is attached as an effector peptide. Between the effector peptide and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, the sequence of the effector peptide has attached at its C-terminus a transporting sequence consisting of 6 arginine residues. Transporting sequence aids in penetration of the cell membrane and transportation of the fusion protein into the cell.

Structure of the fusion protein is shown schematically in FIG. 4 and its amino acid sequence and the DNA encoding sequence comprising codons optimized for expression in E. coil are, respectively, SEQ. No. 17 and SEQ. No. 66 as shown in the attached Sequence Listing.

The amino acid sequence SEQ. No. 17 of the structure described above was used as a template to generate its coding DNA sequence SEQ. No. 66. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 18

### The Fusion Protein of SEQ. No. 18

The protein of SEQ. No. 18 is a fusion protein having the length of 187 amino acids and the mass of 21.7 kDa, in which at the N-terminus of the sequence TRAIL121-281 a 10-amino acid fragment of tubulin (SEQ. No. 38) is attached as an effector peptide. Between the effector peptide and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, the sequence of the effector peptide has attached at its C-terminus a transporting sequence consisting of 6 arginine residues. Transporting sequence aids in penetration of the cell membrane and transportation of the fusion protein into the cell.

Structure of the fusion protein is shown schematically in FIG. 4 and its amino acid sequence and the DNA encoding sequence comprising codons optimized for expression in E. coli are, respectively, SEQ. No. 18 and SEQ. No. 67 as shown in the attached Sequence Listing.

The amino acid sequence SEQ. No. 18 of the structure described above was used as a template to generate its coding DNA sequence SEQ. No. 67. A plasmid containing the coding sequence of DNA was generated and overexpression of the fusion protein was carried out in accordance with the general procedures described above. Overexpression was performed according to the general procedure B, using E. coil B.21 (DE3) or Tuner (DE3) strains from Novagen. The protein was separated by electrophoresis in accordance with the general procedure described above.

### EXAMPLE 19

### The Fusion Protein of SEQ. No. 19

The protein of SEQ. No. 19 is a fusion protein having the length of 196 amino acids and the mass of 22,54 kDa, in which at the N-terminus of the sequence TRAIL121-281 a melittin (SEQ. No. 39) is attached as an effector peptide. Between the effector peptide and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment.

Structure of the fusion protein is shown schematically in FIG. 4 and its amino acid sequence and the DNA encoding sequence comprising codons optimized for expression in E. coil are, respectively, SEQ. No. 19 and SEQ. No. 68 as shown in the attached Sequence Listing.

The amino acid sequence SEQ. No. 19 of the structure described above was used as a template to generate its coding DNA sequence SEQ. No. 68. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 20

### The Fusion Protein of SEQ. No. 20

The protein of SEQ. No. 20 is a fusion protein having the length of 184 amino acids and the mass of 21.4 kDa, in which at the N-terminus of the sequence TRAIL121-281 a 6-amino acid peptide C2 derived from bee defensin (SEQ. No. 40) is attached as an effector peptide. Between the effector peptide and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, the sequence of the effector peptide has attached at its C-terminus a transporting sequence consisting of 6 arginine residues. Transporting sequence aids in penetration of the cell membrane and transportation of the fusion protein into the cell.

Structure of the fusion protein is shown schematically in FIG. 4 and its amino acid sequence and the DNA encoding sequence comprising codons optimized for expression in E. coli are, respectively, SEQ. No. 20 and SEQ. No. 69 as shown in the attached Sequence Listing.

The amino acid sequence SEQ. No. 20 of the structure described above was used as a template to generate its coding DNA sequence SEQ. No. 69. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 21

### The Fusion Protein of SEQ. No. 21

The protein of SEQ. No. 21 is a fusion protein having the length of 189 amino acids and the mass of 21.4 kDa, in which at the N-terminus of the sequence TRAIL121-281 there are attached two repeated sequences of 8-amino acid peptide binding to FGF-2 ligand (SEQ. No. 41) as an effector peptide. Between the effector peptides sequences there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, between the second effector peptide and the sequence of TRAIL domain there is incorporated a linker consisting of two glycine residues which aids in stabilization of trimeric structure.

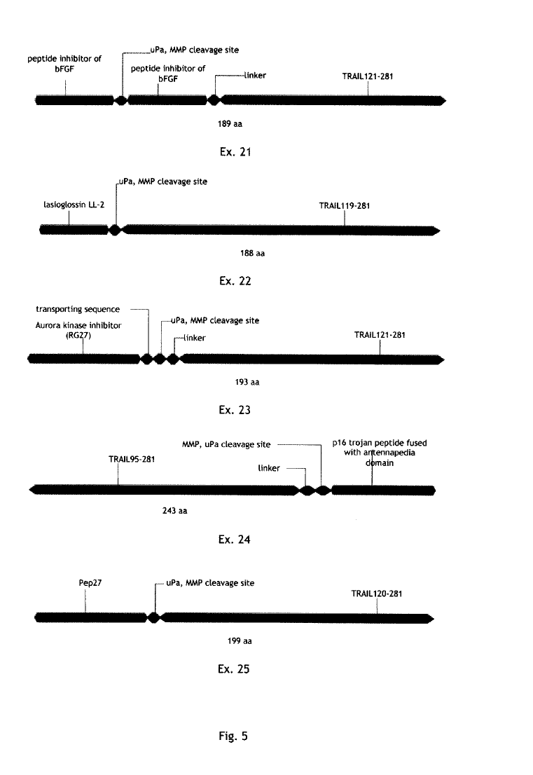

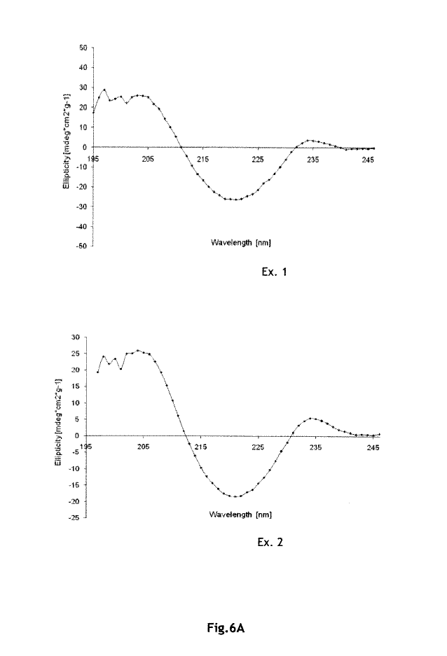

Structure of the fusion protein is shown schematically in FIG. 5 and its amino acid sequence and the DNA encoding sequence comprising codons optimized for expression in E. coli are, respectively, SEQ. No. 21 and SEQ. No. 70 as shown in the attached Sequence Listing.

The amino acid sequence SEQ. No. 21 of the structure described above was used as a template to generate its coding DNA sequence SEQ. No. 70. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 22

### The Fusion Protein of SEQ. No. 22

The protein of SEQ. No. 22 is a fusion protein having the length of 188 amino acids and the mass of 21.6 kDa, in which at the N-terminus of the sequence TRAIL119-281 a 15-amino acid peptide lasioglossin LL2 (SEQ. No. 42) is attached as an effector peptide. Between the effector peptide sequence and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment.

Structure of the fusion protein is shown schematically in FIG. 5 and its amino acid sequence and the DNA encoding sequence comprising codons optimized for expression in E. coli are, respectively, SEQ. No. 22 and SEQ. No. 71 as shown in the attached Sequence Listing.

The amino acid sequence SEQ. No. 22 of the structure described above was used as a template to generate its coding DNA sequence SEQ. No. 71. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 23

### The Fusion Protein of SEQ. No. 23

The protein of SEQ. No. 23 is a fusion protein having the length of 193 amino acids and the mass of 21.6 kDa, in which at the N-terminus of the sequence TRAIL121-281 a 13-amino acid peptide acting as an inhibitor of interactions RasGAP-Aurora B (SEQ. No. 43) is attached as an effector peptide. Between the effector peptide sequence and the TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment. Additionally, the sequence of the effector peptide has attached at its C-terminus a transporting sequence consisting of 8 arginine residues. Transporting sequence aids in penetration of the cell membrane and transportation of the fusion protein into the cell. Additionally, between the sequence of metalloprotease cleavage site and the sequence of TRAIL domain there is incorporated a cysteine is residue which aids in stabilization of trimeric structure.

Structure of the fusion protein is shown schematically in FIG. 5 and its amino acid sequence and the DNA encoding sequence comprising codons optimized for expression in E. coli are, respectively, SEQ. No. 23 and SEQ. No. 72 as shown in the attached Sequence Listing.

The amino acid sequence SEQ. No. 23 of the structure described above was used as a template to generate its coding DNA sequence SEQ. No. 72. A plasmid containing the coding sequence of DNA was generated and overexpression of the fusion protein was carried out in accordance with the general procedures described above. Overexpression was performed according to the general procedure B, using E. coli B.21 (DE3) or Tuner (DE3) strains from Novagen. The protein was separated by electrophoresis in accordance with the general procedure described above.

### EXAMPLE 24

### The Fusion Protein of SEQ. No. 24

The protein of SEQ. No. 24 is a fusion protein having the length of 243 amino acids and the mass of 27.8 kDa, in which at the C-terminus of the sequence TRAIL95-281 a 38-amino acid fragment of p16 peptide fused with a 17-amino-acid transporting domain of antennapedia (SEQ. No. 33) is attached as an effector peptide. Between the effector peptide sequence and the TRAIL domain there are incorporated sequentially next to each other sequences of cleavage sites recognized by metalloprotease MMP (SEQ. No. 45) and urokinase uPA (SEQ. No. 46) due to which the effector peptide will undergo cleavage in the tumor environment. Additionally, between sequence of TRAIL and the sequence of cleavage site recognized by metalloproteinase MMP there is incorporated a flexible cysteine-alanine linker (SEQ. No. 47).

Structure of the fusion protein is shown schematically in FIG. 5 and its amino acid sequence and the DNA encoding sequence comprising codons optimized for expression in E. coil are, respectively, SEQ. No. 24 and SEQ. No. 73 as shown in the attached Sequence Listing.

The amino acid sequence SEQ. No. 24 of the structure described above was used as a template to generate its coding DNA sequence SEQ. No. 73. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

### EXAMPLE 25

### The Fusion Protein of SEQ. No. 25

The protein of SEQ. No. 25 is a fusion protein having the length of 199 amino acids and the mass of 23.4 kDa, in which at the N-terminus of the sequence TRAIL120-281 the analogue of Pep27 peptide (SEQ. No. 44) is attached as the effector peptide. Between the effector peptide sequence and the N-terminus of TRAIL domain there are incorporated sequentially next to each other sequences of cleavage sites recognized by urokinase uPA (SEQ. No. 46) and metalloprotease MMP (SEQ. No. 45) due to which the effector peptide will undergo cleavage in the tumor environment.

Structure of the fusion protein is shown schematically in FIG. 5 and its amino acid sequence and the DNA encoding sequence comprising codons optimized for expression in E. coli are, respectively, SEQ. No. 25 and SEQ. No. 74 as shown in the attached Sequence Listing.

The amino acid sequence SEQ. No. 25 of the structure described above was used as a template to generate its coding DNA sequence SEQ. No. 74. A plasmid containing the coding sequence of DNA was generated and overexpression of the fusion protein was carried out in accordance with the general procedures described above. Overexpression was performed according to the general procedure B, using E. coli BL21 (DE3) or E. coil Tuner (DE3) strain from Novagen. The protein was separated by electrophoresis in accordance with the general procedure described above.

### EXAMPLE 26

### Examination of Anti-Tumor Activity of the Fusion Proteins

Examination of anti-tumor activity of the fusion proteins was carried out in vitro in a cytotoxicity assay on tumor cell lines and in vivo in mice. For comparison purposes, rhTRAIL114-281 protein and placebo were used.

1. Measurement of Circular dichroism

Quality of the preparations of fusion proteins in terms of their structures was determined by circular dichroism (CD) for Ex. 1^{a}, Ex. 2^{a}, and Ex. 8^{a}.

Circular dichroism is used for determination of secondary structures and conformation of proteins. CD method uses optical activity of the protein structures, manifested in rotating the plane of polarization of light and the appearance of elliptical polarization. CD spectrum of proteins in far ultraviolet (UV) provides precise data on the conformation of the main polypeptide chain.

Samples of the protein to be analysed, after formulation into a buffer consisting of 50 mM Tris-HCl pH 8.0, 100 mM NaCl, 10% glycerol, 0.1 mM ZnCl₂, 80 mM saccharose, 5 mM DTT, were dialysed in the dialysis bags (Sigma-Aldrich) with cut-off 12 kDa. Dialysis was performed against 100 fold excess (v/v) of buffer comparing to the protein preparations with stirring for several hours at 4° C. After dialysis was completed, each preparation was centrifuged (25 000 rpm., 10 min., 4° C.) and the appropriate supernatants were collected. Protein concentration in the samples thus obtained was determined by Bradford method.

Measurement of circular dichroism for proteins in the concentration range of 0.1-2.7 mg/ml was performed on Jasco J-710 spectropolarimeter, in a quartz cuvette with optical way 0.2 mm or 1 mm. The measurement was performed under the flow of nitrogen at 7 l/min, which allowed to perform of the measurement in the wavelength range from 195 to 250 nm. Parameters of the measurement: spectral resolution of −1 nm; half width of the light beam 1 nm; sensitivity 20 mdeg, the averaging time for one wavelength −8 s, scan speed 10 nm/min.

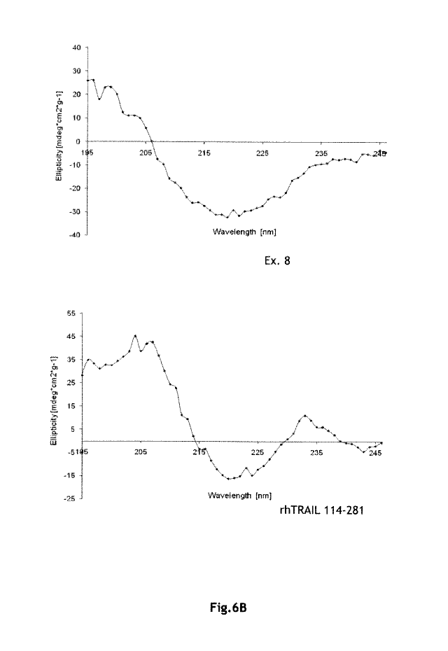

The results were presented as the average of three measurements. Circular dichroism spectra for rhTRAIL114-281 and proteins of Ex. 1^{a}, Ex. 2^{a} and Ex. 8^{a} are presented in FIG. 6.

Obtained spectra were analyzed numerically in the range of 193-250 nm using CDPro software. Points for which the voltage at the photomultiplier exceeded 700 V were omitted, due to too low signal to noise ratio in this wavelength range.

The data obtained served for calculations of particular secondary structures content in the analyzed proteins with use of CDPro software (Table 1).

TABLE 1
Content of secondary structures in the analyzed proteins

| | NRMSD | | | | |
|---|---|---|---|---|---|
| Protein | (Exp-Cal) | α-helix | β-sheet | Schift | Disorder |
| Ex. 1^{a} | 0.205 | 0.6% | 44.1% | 27.3% | 28.0% |
| Ex. 2^{a} | 0.092 | 0.1% | 40.8% | 24.5% | 34.6% |
| Ex. 8^{a} | 0.197 | 4.3% | 32.0% | 25.5% | 38.2% |
| rhTRAIL* | | 1.94% | 50.97% | 7.74% | 39.35% |
| rhTRAIL 114-281 | 0.389 | 4.9% | 33.7% | 23.1% | 38.3% |

*value obtained on the basis of crystalline structure 1D4V

The control molecule (rhTRAIL114-281) shows CD spectrum characteristic for the proteins with predominantly type β-sheet structures (sharply outlined ellipticity minimum at the wavelength of 220 nm). This confirms the calculation of secondary structure components, suggesting a marginal number of a-helix elements.

The obtained result is also consistent with data from the crystal structure of hTRAIL protein, and characteristic for fusion proteins of the invention Ex. 1^{a}, Ex. 2^{a} and Ex., wherein beta elements constitute 32-44% of their structure. In the case of all embodiments, dichroism spectra are characterized one minimum at wavelength 220 nm.

Since the small peptides attached to TRAIL constitute a small portion of the protein and do not need to create a defined secondary structure, analyzed proteins should not differ significantly from the starting protein.

2.Tests on Cell Lines in vitro

Cell Lines

TABLE 2
Adherent cell lines

| | | | number of |
|---|---|---|---|
| | | | cells per |
| | | | well |
| Cell line | Cancer type | Medium | (thousands) |
| Colo 205 | human colorectal | RPMI + 10% FBS + penicillin + | 5 |
| ATCC | cancer | streptomycin | |
| #CCL-222 | | | |
| HT-29 | human colorectal | McCoy's + 10% FBS + penicillin + | 5 |
| ATCC | cancer | streptomycin | |
| # CCL-2 | | | |
| DU-145 | human prostate | RPMI + 10% FBS + penicillin + | 3 |
| ATCC | cancer | streptomycin | |
| # HTB-81 | | | |
| PC-3 | human prostate | RPMI + 10% FBS + penicillin + | 4 |
| ATCC | cancer | streptomycin | |
| # CRL-1435 | | | |
| MCF-7 | human breast cancer | MEM + 10% FBS + penicillin + | 4.5 |
| ATCC | | streptomycin | |
| #HTB-22 | | | |
| MDA-MB-231 | human breast cancer | DMEM + 10% FBS + penicillin + | 4.5 |
| ATCC | | streptomycin | |
| # HTB-26 | | | |
| MDA-MB-435s | human breast cancer | DMEM + 10% FBS + penicillin + | 4 |
| ATCC# HTB-129 | | streptomycin | |
| UM-UC-3 | human bladder | MEM + 10% FBS + penicillin + | 3.5 |
| ATCC | cancer | streptomycin | |
| # CLR-1749 | | | |
| SW780 | human bladder | DMEM + 10% FBS + penicillin + | 3 |
| ATCC | cancer | streptomycin | |
| #CRL-2169 | | | |
| SW620 | human colorectal | DMEM + 10% FBS + penicillin + | 5 |
| ATCC | cancer | streptomycin | |
| #CCL-227 | | | |
| BxPC-3 | human pancreatic | RPMI + 10% FBS + penicillin + | 4.5 |
| ATCC | cancer | streptomycin | |
| #CRL-1687 | | | |
| SK-OV-3 | human ovarian | McCoy's + 10% FBS + penicillin + | 4 |
| ATCC | cancer | streptomycin | |
| # HTB-77 | | | |
| NIH: OVCAR-3 | human ovarian | RPMI + 20% FBS + 0.01 mg/ml | 7 |
| ATCC | cancer | insulina + penicillin + | |
| #HTB-161 | | streptomycin | |
| HepG2 | human liver | MEM + 10% FBS + penicillin + | 7 |
| ATCC | hepatoma | streptomycin | |
| # HB-8065 | | | |
| 293 | Human embrional | MEM + 10% FBS + penicillin + | 4 |
| ATCC | kidney cells | streptomycin | |
| # CLR-1573 | | | |
| ACHN | human kidney cancer | MEM + 10% FBS + penicillin + | 4 |
| ATCC | | streptomycin | |
| #CCL-222 | | | |
| CAKI 1 | human kidney cancer | McCoy's + 10% FBS + penicillin + | 3.5 |
| ATCC | | streptomycin | |
| #HTB-46 | | | |
| CAKI 2 | human kidney cancer | McCoy's + 10% FBS + penicillin + | 3.5 |
| ATCC | | streptomycin | |
| # HTB-47 | | | |
| NCI-H69AR | human small cell | RPMI + 10% FBS + penicillin + | 10 |
| ATCC | lung cancer | streptomycin | |
| #CRL-11351 | | | |
| HT144 | human melanoma | McCoy's + 10% FBS + penicillin + | 7 |
| ATCC | cells | streptomycin | |
| # HTB-63 | | | |
| NCI-H460 | human lung cancer | RPMI + 10% FBS + penicillin + | 2.5 |
| ATCC | | streptomycin | |
| #HTB-177 | | | |
| A549 | human lung cancer | RPMI + 10% FBS + penicillin + | 2.5 |
| ATCC | | streptomycin | |
| # CCL-185 | | | |
| MES-SA | human uterine | McCoy's + 10% FBS + penicillin + | 3.5 |
| ATCC | sarcoma | streptomycin | |
| # CRL-1976 | | | |
| MES-SA/Dx5 | multidrug-resistant | McCoy's + 10% FBS + penicillin + | 4 |
| ATCC | human uterine | streptomycin | |
| #CRL-1977 | sarcoma | | |
| MES-SA/Mx2 | human uterine | Waymouth's MB 752/1 + | 4 |
| ATCC | sarcoma | McCoy's (1:1) + | |
| #CRL-2274 | | 10% FBS + penicillin + | |
| | | streptomycin | |
| SK-MES-1 ATCC | human lung cancer | MEM + 10% FBS + penicillin + | 5 |
| # HTB-58 | | streptomycin | |
| HCT-116 ATCC | human colorectal | McCoy's + 10% FBS + penicillin + | 3 |
| # CCL-247 | cancer | streptomycin | |
| MCF10A ATCC | mammary epithelial | DMEM:F12 + 5% horse plasma + | 5 |
| # CRL-10317 | cells | 0.5 μg/ml hydrocortisone + 10 μg/ml | |
| | | insuline + 20 ng/ml | |
| | | growth factor EGF | |
| Panc-1 CLS | human pancreatic | DMEM + 10% FBS + penicillin + | 5 |
| 330228 | cancer | streptomycin | |
| Panc03.27 | human pancreatic | RPMI + 10% FBS + penicillin + | 5 |
| ATCC | cancer | streptomycin | |
| # CRL-2549 | | | |
| PLC/PRF/5 CLS | human liver | DMEM + 10% FBS + penicillin + | 5 |
| 330315 | hepatoma | streptomycin | |
| LNCaP | human prostate | RPMI + 10% FBS + penicillin + | 4.5 |
| ATCC | cancer | streptomycin | |
| # CRL-1740 | | | |
| SK-Hep-1 | human liver | RPMI + 10% FBS + penicillin + | 10 |
| CLS300334 | hepatoma | streptomycin | |
| A498 | human kidney cancer | MEM + 10% FBS + penicillin + | 3 |
| CLS 300113 | | streptomycin | |
| HT1080 ATCC | Human fibrosarcoma | MEM + 10% FBS + penicillin + | 3 |
| #CCL-121 | | streptomycin | |

TABLE 3
Nonadherent cells:

| | | | number of |
|---|---|---|---|
| | | | cells per |
| | | | well |
| Cell line | Cancer type | Medium | (thousands) |
| NCI-H69 | human small cell | RPMI + 10% FBS + penicillin + | 22 |
| ATCC # HTB-119 | lung cancer | streptomycin | |
| Jurkat A3 | human leukaemia | RPMI + 10% FBS + penicillin + | 10 |
| ATCC #CRL-2570 | | streptomycin | |
| HL60 | human leukaemia | RPMI + 20% FBS + penicillin + | 10 |
| ATCC # CCL-240 | | streptomycin | |
| CCRF-CEM | human leukaemia | RPMI + 20% FBS + penicillin + | 10 |
| ATCC # CCL-119 | | streptomycin | |

MTT Cytotoxicity Test

MTT assay is a colorimetric assay used to measure proliferation, viability and cytotoxicity of cells. It consists in decomposition of a yellow tetrazolium salt MTT (4,5-dimethyl-2-thiazolyl)-2,5-diphenyltetrazolium bromide) to the water-insoluble purple dye formazan by mitochondrial enzyme succinate-tetrazolium reductase 1. MTT reduction occurs only in living cells. Data analysis consists in determining IC₅₀ concentration of the protein (in ng/ml), at which the 50% reduction in the number of cells occurs in the population treated compared to control cells. Results were analyzed using GraphPad Prism 5.0 software. The test was performed according to the literature descriptions (Cells, J E, (1998). Cell Biology, a Laboratory Handbook, second edition, Academic Press, San Diego; Yang, Y., Koh, L W, Tsai, J H., (2004); Involvement of viral and chemical factors with oral cancer in Taiwan, Jpn J Clin Oncol, 34 (4), 176-183). Cell culture medium was diluted to a defined density (10⁴-10⁵ cells per 100 μl). Then 100 μl of appropriately diluted cell suspension was applied to a 96-well plate in triplicates. Thus prepared cells were incubated for 24 h at 37° C. in 5% or 10% CO₂, depending on the medium used, and then to the cells (in 100 μl of medium) further 100 μl of the medium containing various concentrations of tested proteins were added. In the case of combination hTRAIL114-281 and p21WAF effector protein, 100 μl of the medium containing mixture of hTRAIL114-281 and p21WAF effector protein in molar ratio 1:1 was added. After incubation of the cells with tested proteins over the period of next 72 hours, which is equivalent to 3-4 times of cell division, the medium with the test protein was added with 20 ml of MTT working solution [5 mg/ml], and incubation was continued for 3 h at 37° C. in 5% CO₂. Then the medium with MTT solution was removed, and formazan crystals were dissolved by adding 100 μl of DMSO. After stirring, the absorbance was measured at 570 nm (reference filter 690 nm).

EZ4U Cytotoxicity Test

EZ4U (Biomedica) test was used for testing cytotoxic activity of the proteins in nonadherent cell lines. The test is a modification of the MTT method, wherein formazan formed in the reduction of tetrazolium salt is water-soluble. Cell viability study was carried out after continuous 72-hour incubation of the cells with protein (seven concentrations of protein, each in triplicates). On this basis IC₅O values were determined (as an average of two independent experiments) using the GraphPad Prism 5 software. Control cells were incubated with the solvent only.

The results of in vitro cytotoxicity tests are summarized as IC₅₀ values (ng/ml), which corresponds to the protein concentration at which the cytotoxic effect of fusion proteins is observed at the level of 50% with respect to control cells treated only with solvent. Each experiment represents the average value of at least two independent experiments performed in triplicates. As a criterion of lack of activity of protein preparations the IC₅₀ limit of 2000 ng/ml was adopted. Fusion proteins with an IC₅₀ value above 2000 were considered inactive.

Cells selected for this test included tumor cell lines that are naturally resistant to TRAIL protein (the criterion of natural resistance to TRAIL: IC₅₀ for TRAIL protein >2000), as well as tumor cell lines sensitive to TRAIL protein and resistant to doxorubicin line MES-SA/DX5 as a cancer line resistant to conventional anticancer medicaments.

Undifferentiated HUVEC cell line was used as a healthy control cell line for assessment of the effect/toxicity of the fusion proteins in non-cancer cells.

The results obtained confirm the possibility of overcoming the resistance of the cell lines to TRAIL by administration of certain fusion proteins of the invention to cells naturally resistant to TRAIL. When fusion proteins of the invention were administered to the cells sensitive to TRAIL, in some cases a clear and strong potentiation of the potency of action was observed, which was manifested in reduced IC₅₀ values of the fusion protein compared with IC₅₀ for the TRAIL alone. Furthermore, cytotoxic activity of the fusion protein of the invention in the cells resistant to classical anti-cancer medicament doxorubicin was obtained, and in some cases it was stronger than activity of TRAIL alone.

The IC₅₀ values above 2000 obtained for the non-cancer cell lines show the absence of toxic effects associated with the use of proteins of the invention for healthy cells, which indicates potential low systemic toxicity of the protein.

The results obtained for combination of hTRAIL114-281 and p21WAF effector peptide consisting of mixture of hTRAIL114-281 and 20-amino acid p21WAF derived effector peptide (custom solid phase synthesis) in molar ratio 1:1, compared with results obtained for fusion protein of Ex. 8^{b} (comprising hTRAIL121-281 and 20-amino acid p21WAF derived effector peptide) and with 62 results obtained for single molecule of hTRAIL114-281 and single molecule of p21WAF derived effector peptide revealed the advantageous properties of the fusion protein over its single constituents and combination thereof.

The fusion protein of Ex. 8^{b} overcomes the resistance to TRAIL of A549 cell line. In the case of TRAIL sensitive cell lines the fusion protein of Ex. 8^{b} reveals higher cytotoxic activity than single molecules of hTRAIL114-281 and p21WAF derived peptide.

Determination of Cytotoxic Activity of Selected Protein Preparations Against Extended Panel of Tumor Cell Lines

Table 4 presents the results of the tests of cytotoxic activity in vitro for selected fusion proteins of the invention against a broad panel of tumor cells from different organs, corresponding to the broad range of most common cancers.

The experimental results are presented as a mean value±standard deviation (SD). All calculations and graphs were prepared using the GraphPad Prism 5.0 software.

Obtained IC₅₀ values confirm high cytotoxic activity of fusion proteins and thus their potential utility in the treatment of cancer.

TABLE 4
Analysis of cytotoxic activity of selected protein preparations against broad panel of tumor cell lines

| | A549 | | NCI-H460 | | HT 29 | | MCF10A | | HCT116 | | MES-SA | | MES-SA/Dx5 | | SK-MES-1 | |
|---|---|---|---|---|---|---|---|---|---|---|---|---|---|---|---|---|
| cell line | mean | SD | mean | SD | mean | SD | mean | SD | mean | SD | mean | SD | mean | SD | mean | SD |
| rhTRAIL | 10000 | | 5888.50 | 111.02 | 10000 | | 10000 | | 7558 | | | | 29.15 | 12.66 | 33.60 | |
| 95-281 | | | | | | | | | | | | | | | | |
| Ex. 2^{a} | 10000 | | 4246 | 26.16 | 10000 | | 10000 | | 60.87 | 15.26 | 5000 | | 18.96 | 6.025 | 18.88 | 5.063 |
| | OVCAR-3 | | UM-UC-3 | | | | | | | | | | | | | |
| cell line | mean | SD | mean | SD | | | | | | | | | | | | |
| rhTRAIL | 963 | 144.25 | 2242 | 963 | | | | | | | | | | | | |
| 95-281 | | | | | | | | | | | | | | | | |
| Ex. 2^{a} | 26.1 | | 35.06 | 26.1 | | | | | | | | | | | | |
| | A549 | | HT 29 | | MCF10A | | HCT116 | | MES-SA | | MES-SA/Dx5 | | SK-MES-1 | | MDA-MB-231 | |
| cell line | mean | SD | mean | SD | mean | SD | mean | SD | mean | SD | mean | SD | mean | SD | mean | SD |
| rhTRAIL | 10000 | | 10000 | | 10000 | | 7558 | | | | 29.15 | 12.66 | 33.60 | | 10000 | |
| 95-281 | | | | | | | | | | | | | | | | |
| Ex. 3^{a} | 5000 | | 2000 | | 5000 | | 41.00 | 2.737 | 36.18 | 17.42 | 34.31 | 9.631 | 7.755 | 3.147 | 20.86 | |
| | OVCAR-3 | | UM-UC-3 | | | | | | | | | | | | | |
| cell line | mean | SD | mean | SD | | | | | | | | | | | | |
| rhTRAIL | 963 | 144.25 | 2242 | 963 | | | | | | | | | | | | |
| 95-281 | | | | | | | | | | | | | | | | |
| Ex. 3^{a} | 27.51 | | 20.40 | 27.51 | | | | | | | | | | | | |
| | A549 | | MCF10A | | HCT116 | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 10000 | | 7558 | | | | 29.15 | 12.66 | 33.60 | | | | | |
| 95-281 | | | | | | | | | | | | | | | | |
| Ex. 5^{a} | 3000 | | 8949 | 1487 | 179.8 | 158.7 | 95.00 | 38.75 | 11.16 | 2.577 | 79.65 | 10.68 | | | | |
| | A549 | | HCT116 | | MCF10A | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 7558 | | 10000 | | | | 29.15 | 12.66 | 33.60 | | | | | |
| 95-281 | | | | | | | | | | | | | | | | |
| Ex. 15^{a} | 1000 | | 603.5 | 560.8 | 1000 | | 123.0 | 42.06 | 328.9 | 125.4 | 78.40 | 7.297 | | | | |
| | A549 | | HCT116 | | MCF10A | | MES-SA | | MES-SA/Dx5 | | SK-MES-1 | | HT29 | | | |
| cell line | mean | SD | mean | SD | mean | SD | mean | SD | mean | SD | mean | SD | mean | SD | | |
| rhTRAIL | 10000 | | 7558 | | 10000 | | | | 29.15 | 12.66 | 33.60 | | 10000 | 17.68 | | |
| 95-281 | | | | | | | | | | | | | | | | |
| Ex. 17^{a} | 10000 | | 128.4 | 18.10 | 10000 | | 5000 | | 44.26 | 7.241 | 81.19 | | 7818 | 1599 | | |
| | A549 | | HCT116 | | MCF10A | | MES-SA | | MES-SA/Dx5 | | SK-MES-1 | | COLO 205 | | DU 145 | |
| cell line | mean | SD | mean | SD | mean | SD | mean | SD | mean | SD | mean | SD | mean | SD | mean | SD |
| rhTRAIL | 10000 | | 7558 | | 10000 | | | | 29.15 | 12.66 | 33.60 | | 24.90 | 17.68 | 10000 | |
| 95-281 | | | | | | | | | | | | | | | | |
| Ex. 8^{a} | 91.050 | 5.45 | 51.720 | 35.470 | 218.00 | 131.50 | 8.841 | 0.1563 | 1.662 | 0.2623 | 3.929 | 0.4363 | 0.03 | 0.03 | 22.90 | 18.24 |
| | MCF 7 | | MDA-MB-231 | | PC 3 | | SW620 | | SW780 | | UM-UC-3 | | HT29 | | 293 | |
| cell line | mean | SD | mean | SD | mean | SD | mean | SD | mean | SD | mean | SD | mean | SD | mean | SD |
| rhTRAIL | 10000 | | 10000 | 0.00 | 10000 | 0.00 | 10000 | 0.00 | 120.00 | 42.43 | 2241 | | 10000 | | 10000 | |
| 95-281 | | | | | | | | | | | | | | | | |
| Ex. 8^{a} | 8500 | | 2.417 | 0.62 | 8500 | | 8500 | | 0.02 | 0.02 | 0.79 | 0.04 | 4404 | 571.34 | 8500 | |
| | ACHN | | CAKI 2 | | SK-OV-3 | | BxPC3 | | H69AR | | HepG2 | | HT 144 | | NCI-H460 | |
| cell line | mean | SD | mean | SD | mean | SD | mean | SD | mean | SD | mean | SD | mean | SD | mean | SD |
| rhTRAIL | 10000 | | 10000 | | 10000 | | 64.710 | 31.81 | 10000 | | 10000 | | 1733.5 | 218.5 | 5888.5 | 111.02 |
| 95-281 | | | | | | | | | | | | | | | | |
| Ex. 8^{a} | 6.02 | 0.16 | 36.44 | 2.32 | 8500 | | 0.653 | 0.08 | 8500 | | 8500 | | 0.27 | 0.02 | 3.06 | 0.76 |
| | LNCaP | | OV-CAR-3 | | NCI-H69 | | JURKAT A3 | | HL60 | | CCRF-CEM | | | | | |
| cell line | mean | SD | mean | SD | mean | SD | mean | SD | mean | SD | mean | SD | | | | |
| rhTRAIL | 2051.50 | 465.98 | 963 | 144.25 | 10000 | | 10000 | 0.00 | 10000 | | 10000 | | | | | |
| 95-281 | | | | | | | | | | | | | | | | |
| Ex. 8^{a} | 8500 | | 0.52 | 0.48 | 8500 | | 1.082 | 1.50 | 8500 | | 8500 | | | | | |
| | A549 | | MCF10A | | HCT116 | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 10000 | | 7558 | | | | 29.15 | 12.66 | 33.60 | | | | | |
| 95-281 | | | | | | | | | | | | | | | | |
| Ex. 16^{a} | 42.85 | 6.718 | 92.28 | 42.03 | 15.85 | 5.586 | 8.460 | 1.243 | 1.421 | 0.1336 | 3.045 | 0.8132 | | | | |
| | COLO 205 | | DU 145 | | MCF 7 | | MDA-MB-231 | | PC 3 | | SW620 | | SW780 | | UM-UC-3 | |
| cell line | mean | SD | mean | SD | mean | SD | mean | SD | mean | SD | mean | SD | mean | SD | mean | SD |
| rhTRAIL | 24.90 | 17.68 | 10000 | | 10000 | | 10000 | 0.00 | 10000 | 0.00 | 10000 | 0.00 | 120 | 42.43 | 2241 | |
| 95-281 | | | | | | | | | | | | | | | | |
| Ex. 19^{a} | 83.90 | 10.04 | 900 | 141 | 2250 | 353 | 1141 | 115 | 2500 | | 2299 | 283 | 108 | 0.71 | 179.45 | 27.51 |
| | HT29 | | 293 | | ACHN | | CAKI 2 | | SK-OV-3 | | BxPC3 | | H69AR | | HepG2 | |
| cell line | mean | SD | mean | SD | mean | SD | mean | SD | mean | SD | mean | SD | mean | SD | mean | SD |
| rhTRAIL | 10000 | | 10000 | | 10000 | | 10000 | | 10000 | | 64.710 | 31.81 | 10000 | | 10000 | |
| 95-281 | | | | | | | | | | | | | | | | |
| Ex. 19^{a} | 2500 | | 2500 | | 919 | 271.53 | 2180.5 | 451.84 | 2500 | | 279.45 | 30.76 | 2500 | | 2213.5 | 33.23 |
| | HT 144 | | NCI-H460 | | LNCaP | | OV-CAR-3 | | NCI-H69 | | JURKAT A3 | | HL60 | | CCRF-CEM | |
| cell line | mean | SD | mean | SD | mean | SD | mean | SD | mean | SD | mean | SD | mean | SD | mean | SD |
| rhTRAIL | 1733.5 | 218.5 | 5888.5 | 111.02 | 2051.50 | 465.98 | 963 | 144.25 | 10000 | 10000 | 10000 | 10000 | | | | |
| 95-281 | | | | | | | | | | | | | | | | |
| Ex. 19^{a} | 94.25 | 4.96 | 102.19 | 5.40 | 179 | 103.24 | 281.20 | 35.07 | 2500 | | 443.05 | 72.2 | 2500 | | 2500 | |
| | A549 | | HCT116 | | MCF10A | | MES-SA/Dx5 | | HT29 | | | | | | | |
| cell line | mean | SD | mean | SD | mean | SD | mean | SD | mean | SD | | | | | | |
| rhTRAIL | 10000 | | 7558 | | 10000 | | 29.15 | 12.66 | 10000 | 17.68 | | | | | | |
| 95-281 | | | | | | | | | | | | | | | | |
| Ex. 17^{a} | 2887 | 773.6 | 574.9 | 49.29 | 10000 | | 75.96 | 11.82 | 6808 | 2292 | | | | | | |
| | A549 | | NCI-H460 | | HT 29 | | SW620 | | PLC/PRF/5 | | HepG2 | | PANC-1 | | MCF10A | |
| cell line | mean | SD | mean | SD | mean | SD | mean | SD | mean | SD | mean | SD | mean | SD | mean | SD |
| rhTRAIL | 10000 | | 5888 | 111 | 10000 | | 10000 | | 10000 | | 10000 | | 10000 | 12.66 | 33.60 | |
| 95-281 | | | | | | | | | | | | | | | | |
| Ex. 23^{a} | 278.4 | | 7.11 | | 10000 | | 11.92 | | 6.821 | | 10.49 | | 22.9 | | 5.196 | |
| | A549 | | NCI-H460 | | HT 29 | | MCF10A | | HCT116 | | MES-SA | | MES-SA/Dx5 | | SK-MES-1 | |
| cell line | mean | SD | mean | SD | mean | SD | mean | SD | mean | SD | mean | SD | mean | SD | mean | SD |
| rhTRAIL | 10000 | | 5888 | 111 | 10000 | | 10000 | | 7558 | | | | 29.15 | 12.66 | 33.60 | |
| 95-281 | | | | | | | | | | | | | | | | |
| Ex. 2^{a} | 10000 | | 4246 | 26.16 | 10000 | | 10000 | | 60 | 15 | 5000 | | 18.96 | 6.025 | 18.88 | 5.063 |
| | OVCAR-3 | | UM-UC-3 | | | | | | | | | | | | | |
| cell line | mean | SD | mean | SD | | | | | | | | | | | | |
| rhTRAIL | 963 | 144.25 | 2242 | 963 | | | | | | | | | | | | |
| 95-281 | | | | | | | | | | | | | | | | |
| Ex. 2^{a} | 26.1 | | 35.06 | 26.1 | | | | | | | | | | | | |
| | A549 | | HT 29 | | MCF10A | | HCT116 | | MES-SA | | MES-SA/Dx5 | | SK-MES-1 | | MDA-MB-231 | |
| cell line | mean | SD | mean | SD | mean | SD | mean | SD | mean | SD | mean | SD | mean | SD | mean | SD |
| rhTRAIL | 10000 | | 10000 | | 10000 | | 7558 | | | | 29.15 | 12.66 | 33.60 | | 10000 | |
| 95-281 | | | | | | | | | | | | | | | | |
| Ex. 3^{a} | 5000 | | 2000 | | 5000 | | 41.00 | 2.737 | 36.18 | 17.42 | 34.31 | 9.631 | 7.755 | 3.147 | 20.86 | |
| | OVCAR-3 | | UM-UC-3 | | | | | | | | | | | | | |
| cell line | mean | SD | mean | SD | | | | | | | | | | | | |
| rhTRAIL | 963 | 144.25 | 2242 | 963 | | | | | | | | | | | | |
| 95-281 | | | | | | | | | | | | | | | | |
| Ex. 3^{a} | 27.51 | | 20.40 | 27.51 | | | | | | | | | | | | |
| | A549 | | MCF10A | | HCT116 | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 10000 | | 7558 | | | | 29.15 | 12.66 | 33.60 | | | | | |
| 95-281 | | | | | | | | | | | | | | | | |
| Ex. 5^{a} | 3000 | | 8949 | 1487 | 179.8 | 158.7 | 95.00 | 38.75 | 11.16 | 2.577 | 79.65 | 10.68 | | | | |
| | A549 | | HCT116 | | MCF10A | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 7558 | | 10000 | | | | 29.15 | 12.66 | 33.60 | | | | | |
| 95-281 | | | | | | | | | | | | | | | | |
| Ex. 15^{a} | 1000 | | 603.5 | 560.8 | 1000 | | 123.0 | 42.06 | 328.9 | 125.4 | 78.40 | 7.297 | | | | |
| | A549 | | HCT116 | | MCF10A | | MES-SA | | MES-SA/Dx5 | | SK-MES-1 | | HT29 | | | |
| cell line | mean | SD | mean | SD | mean | SD | mean | SD | mean | SD | mean | SD | mean | SD | | |
| rhTRAIL | 10000 | | 7558 | | 10000 | | | | 29.15 | 12.66 | 33.60 | | 10000 | 17.68 | | |
| 95-281 | | | | | | | | | | | | | | | | |
| Ex. 17^{a} | 10000 | | 128.4 | 18.10 | 10000 | | 5000 | | 44.26 | 7.241 | 81.19 | | 7818 | 1599 | | |
| | A549 | | HCT116 | | MCF10A | | MES-SA | | MES-SA/Dx5 | | SK-MES-1 | | COLO 205 | | DU 145 | |
| cell line | mean | SD | mean | SD | mean | SD | mean | SD | mean | SD | mean | SD | mean | SD | mean | SD |
| rhTRAIL | 10000 | | 7558 | | 10000 | | | | 29.15 | 12.66 | 33.60 | | 24.90 | 17.68 | 10000 | |
| 95-281 | | | | | | | | | | | | | | | | |
| Ex. 8^{a} | 91.05 | 5.445 | 51.720 | 35.47 | 218.00 | 131.50 | 8.841 | 0.1563 | 1.662 | 0.2623 | 3.929 | 0.4363 | 0.03 | 0.03 | 22.90 | 18.24 |
| | MCF 7 | | MDA-MB-231 | | PC 3 | | SW620 | | SW780 | | UM-UC-3 | | HT29 | | 293 | |
| cell line | mean | SD | mean | SD | mean | SD | mean | SD | mean | SD | mean | SD | mean | SD | mean | SD |
| rhTRAIL | 10000 | | 10000 | 0 | 10000 | 0.00 | 10000 | 0.00 | 120.00 | 42.43 | 2241 | | 10000 | | 10000 | |
| 95-281 | | | | | | | | | | | | | | | | |
| Ex. 8^{a} | 8500 | | 2.417 | 0.62 | 8500 | | 8500 | | 0.02 | 0.02 | 0.79 | 0.04 | 4404 | 571.34 | 8500 | |
| | ACHN | | CAKI 2 | | SK-OV-3 | | BxPC3 | | H69AR | | HepG2 | | HT 144 | | NCI-H460 | |
| cell line | mean | SD | mean | SD | mean | SD | mean | SD | mean | SD | mean | SD | mean | SD | mean | SD |
| rhTRAIL | 10000 | | 10000 | | 10000 | | 64.710 | 31.81 | 10000 | | 10000 | | 1733.5 | 218.5 | 5888.5 | 111.02 |
| 95-281 | | | | | | | | | | | | | | | | |
| Ex. 8^{a} | 6.02 | 0.16 | 36.44 | 2.32 | 8500 | | 0.653 | 0.08 | 8500 | | 8500 | | 0.27 | 0.02 | 3.06 | 0.76 |
| | LNCaP | | OV-CAR-3 | | NCI-H69 | | JURKAT A3 | | HL60 | | CCRF-CEM | | | | | |
| cell line | mean | SD | mean | SD | mean | SD | mean | SD | mean | SD | mean | SD | | | | |
| rhTRAIL | 2051.50 | 465.98 | 963 | 144.25 | 10000 | | 10000 | 0.00 | 10000 | | 10000 | | | | | |
| 95-281 | | | | | | | | | | | | | | | | |
| Ex. 8^{a} | 8500 | | 0.52 | 0.48 | 8500 | | 1.082 | 1.50 | 8500 | | 8500 | | | | | |
| | A549 | | MCF10A | | HCT116 | | MES-SA | | MES-SA/Dx5 | | SK-MES-1 | | | | | |
| cell line | mean | SD | mean | SD | mean | SD | mean | SD | mean | SD | mean | SD | | | | |
| rhTRAIL | 10000 | | 10000 | | 7558 | | | | 29.15 | 12.66 | 33.60 | | | | | |
| 95-281 | | | | | | | | | | | | | | | | |
| Ex. 16^{a} | 42.85 | 6.718 | 92.28 | 42.03 | 15.85 | 5.586 | 8.460 | 1.243 | 1.421 | 0.1336 | 3.045 | 0.8132 | | | | |
| | COLO 205 | | DU 145 | | MCF 7 | | MDA-MB-231 | | PC 3 | | SW620 | | SW780 | | UM-UC-3 | |
| cell line | mean | SD | mean | SD | mean | SD | mean | SD | mean | SD | mean | SD | mean | SD | mean | SD |
| rhTRAIL | 24.90 | 17.68 | 10000 | | 10000 | | 10000 | 0.00 | 10000 | 0.00 | 10000 | 0.00 | 120.00 | 42.43 | 2241 | |
| 95-281 | | | | | | | | | | | | | | | | |
| Ex. 19^{a} | 83.90 | 10.04 | 900 | 141.42 | 2250 | 353.55 | 1141.50 | 115.26 | 2500 | | 2299.5 | 283.55 | 108.10 | 0.71 | 179.45 | 27.51 |
| | HT29 | | 293 | | ACHN | | CAKI 2 | | SK-OV-3 | | BxPC3 | | H69AR | | HepG2 | |
| cell line | mean | SD | mean | SD | mean | SD | mean | SD | mean | SD | mean | SD | mean | SD | mean | SD |
| rhTRAIL | 10000 | | 10000 | | 10000 | | 10000 | | 10000 | | 64.710 | 31.81 | 10000 | | 10000 | |
| 95-281 | | | | | | | | | | | | | | | | |
| Ex. 19^{a} | 2500 | | 2500 | | 919 | 271.53 | 2180.5 | 451.84 | 2500 | | 279.45 | 30.76 | 2500 | | 2213.5 | 33.23 |
| | HT 144 | | NCI-H460 | | LNCaP | | OV-CAR-3 | | NCI-H69 | | JURKAT A3 | | HL60 | | CCRF-CEM | |
| cell line | mean | SD | mean | SD | mean | SD | mean | SD | mean | SD | mean | SD | mean | SD | mean | SD |
| rhTRAIL | 1733.5 | 218.5 | 5888.5 | 111.02 | 2051.50 | 465.98 | 963 | 144.25 | 10000 | | 10000 | | 10000 | | 10000 | |
| 95-281 | | | | | | | | | | | | | | | | |
| Ex. 19^{a} | 94.25 | 4.96 | 102.19 | 5.40 | 179 | 103.24 | 281.20 | 35.07 | 2500 | | 443.05 | 72.2 | 2500 | | 2500 | |
| | A549 | | HCT116 | | MCF10A | | MES-SA/Dx5 | | HT29 | | | | | | | |
| cell line | mean | SD | mean | SD | mean | SD | mean | SD | mean | SD | | | | | | |
| rhTRAIL | 10000 | | 7558 | | 10000 | | 29.15 | 12.66 | 10000 | 17.68 | | | | | | |
| 95-281 | | | | | | | | | | | | | | | | |
| Ex. 17^{a} | 2887 | 773.6 | 574.9 | 49.29 | 10000 | | 75.96 | 11.82 | 6808 | 2292 | | | | | | |
| | A549 | | NCI-H460 | | HT 29 | | SW620 | | PLC/PRF/5 | | HepG2 | | PANC-1 | | MCF10A | |
| cell line | mean | SD | mean | SD | mean | SD | mean | SD | mean | SD | mean | SD | mean | SD | mean | SD |
| rhTRAIL | 10000 | | 5888.50 | 111.02 | 10000 | | 10000 | | 10000 | | 10000 | | 10000 | 12.66 | 33.60 | |
| 95-281 | | | | | | | | | | | | | | | | |
| Ex. 23^{a} | 358.9 | | 7.11 | | 10000 | | 11.92 | | 6.821 | | 10.49 | | 22.9 | | 5.196 | |

| Continuous incubation of preparations with cells over 72 h (test MTT, ng/ml) | | | | | |
|---|---|---|---|---|---|
| MES-SA | MES-SA/Dx5 | HCT116 | SK-MES-1 | A549 | MCF10A |

| Protein | IC₅₀ | ±SD | IC₅₀ | ±SD | IC₅₀ | ±SD | IC₅₀ | ±SD | IC₅₀ | ±SD | IC₅₀ | ±SD |
|---|---|---|---|---|---|---|---|---|---|---|---|---|
| TRAIL 95-281 | | | | | >2000 | | | | >2000 | | | |
| Ex. 11^{a} | 104.4 | | 8.85 | | 84.29 | | 33.48 | | 257 | | 133.3 | |
| Ex. 19^{a} | 217.9 | 85.5 | 132 | 46.7 | 401.4 | 263.9 | 130.5 | 12.7 | >2000 | | >2000 | |
| Ex. 23^{a} | 0.61 | 0.3 | 0.13 | | 1.48 | | 0.42 | | 238.7 | 56.1 | 5.98 | 1.22 |
| Ex. 4^{a} | 205.3 | | 11.36 | 1.51 | 32.25 | | 10.11 | 2.83 | >2000 | | >2000 | |
| Ex. 7^{a} | >2000 | | 22.11 | | 63.5 | | 36.6 | | >2000 | | >2000 | |
| Ex. 14^{a} | 231.2 | 32.7 | 13.73 | 8.6 | 123.5 | 89.7 | 28.48 | 4.82 | 1767 | 822 | >2000 | |
| Ex. 8^{b} | 0.25 | | <0.01 | | 1.08 | 0.52 | <0.01 | | 13.79 | 2.46 | 6.33 | |

| Continuous incubation of preparations with cells over 72 h (test MTT, ng/ml) | | | | | |
|---|---|---|---|---|---|
| MES-SA/Mx25 | COLO205 | DU145 | MCF 7 | MDA-MB-231 | MDA-MB-435s |

| Protein | IC₅₀ | ±SD | IC₅₀ | ±SD | IC₅₀ | ±SD | IC₅₀ | ±SD | IC₅₀ | ±SD | IC₅₀ | ±SD |
|---|---|---|---|---|---|---|---|---|---|---|---|---|
| TRAIL 95-281 | 38.95 | 6.14 | 59.02 | 21.16 | >2000 | | >2000 | | >2000 | | >2000 | |
| Ex. 8^{b} | <0.0001 | | 0.03 | 0.03 | 22.9 | 18.24 | 1548 | 5.66 | 2.42 | 0.62 | <0.01 | |

| | | | | | |
|---|---|---|---|---|---|
| SW620 | SW780 | UM-UC-3 | HT29 | ACHN | CAKI 1 |

| | IC₅₀ | ±SD | IC₅₀ | ±SD | IC₅₀ | ±SD | IC₅₀ | ±SD | IC₅₀ | ±SD | IC₅₀ | ±SD |
|---|---|---|---|---|---|---|---|---|---|---|---|---|
| TRAIL 95-281 | >2000 | | 120 | 42.43 | >2000 | | >2000 | | >2000 | | 395.3 | |
| Ex. 8^{b} | 2645 | 18.38 | 0.02 | 0.02 | 0.79 | 0.04 | 1357 | 328.7 | 2.09 | 0.16 | 3.26 | 0.3 |

| | | | | | |
|---|---|---|---|---|---|
| CAKI 2 | SK-OV-3 | BxPC3 | HepG2 | HT144 | OV-CAR-3 |

| | IC₅₀ | ±SD | IC₅₀ | ±SD | IC₅₀ | ±SD | IC₅₀ | ±SD | IC₅₀ | ±SD | IC₅₀ | ±SD |
|---|---|---|---|---|---|---|---|---|---|---|---|---|
| TRAIL 95-281 | >2000 | | >2000 | | 60.61 | 22.78 | >2000 | | 34.1 | | 963 | 144.25 |
| Ex. 8^{b} | 3.47 | 0.69 | 453.2 | | 0.1 | 0.01 | 105.14 | 73.91 | 0.094 | | 0.52 | 0.48 |

| Continuous incubation of preparations with cells over 72 h (test MTT, ng/ml) | | | | | |
|---|---|---|---|---|---|
| JURKAT A3 | Panc-1 | Panc03.27 | SK Hep-1 | PLC/PRF/5 | A498 |

| Protein | IC₅₀ | ±SD | IC₅₀ | ±SD | IC₅₀ | ±SD | IC₅₀ | ±SD | IC₅₀ | ±SD | IC₅₀ | ±SD |
|---|---|---|---|---|---|---|---|---|---|---|---|---|
| rhTRAIL 95-281 | >2000 | | >2000 | | 315 | | >2000 | | >2000 | | 1611 | 102.53 |
| Ex. 8^{b} | 1.08 | 1.5 | 2.24 | 0.21 | 5.73 | 1.53 | 4.24 | 2.88 | 7.22 | 7.07 | 0.039 | |

| | | | | |
|---|---|---|---|---|
| A5490 | HCT116 | NCI-H460 | HT1080 | NCI-H460 |

| | IC₅₀ | ±SD | IC₅₀ | ±SD | IC₅₀ | ±SD | | IC₅₀ | ±SD | IC₅₀ | ±SD |
|---|---|---|---|---|---|---|---|---|---|---|---|
| rhTRAIL114-281 | >10000 | | 25.57 | 0.93 | 69.68 | 2.55 | rhTRAIL | >2000 | | 438.2 | 77.2 |
| | | | | | | | 95-281 | | | | |
| peptide derived | >1000000 | | >1000000 | | >1000000 | | Ex. 8^{b} | 26.94 | 37.14 | 0.31 | 0.06 |
| from p21WAF | | | | | | | | | | | |
| protein (SEQ. No. | | | | | | | | | | | |
| 29) | | | | | | | | | | | |
| Ex. 8^{b} | 13.79 | 2.46 | 1.08 | 0.52 | 0.31 | 0.06 | Ex. 7^{a} | | | 105 | |
| Mixture of | >10000 | | 15.12 | | 16.88 | | Ex. 14^{a} | | | 49 | 7.05 |
| rhTRAIL114-281: | | | | | | | | | | | |
| peptide derived | | | | | | | | | | | |
| from p21WAF | | | | | | | | | | | |
| protein (SEQ. No. | | | | | | | | | | | |
| 29) 1:1 | | | | | | | | | | | |
| | | | | | | | Ex. 11^{a} | | | 63 | |

2. Antitumor Effectiveness of Fusion Proteins in vivo on Xenografts

Antitumor activity of protein preparations was tested in a mouse model of human colon cancer HCT116, human colon cancer Colo205, human colon cancer model SW620, human liver cancer model HepG2, and human lung cancer models NCI-H460 and NCI-H460-Luc2.

Proteins tested for antitumor activity on xenografts originally expressed with histidine tag that was subsequently removed are designated as a) at the Ex. No. Proteins that were originally expressed without histidine tag are designated as b) at the Ex. No.

Cells

The HCT116 (in mice Crl:CD1-Foxn1^{nu} 1), Colo205, NCI-H460, NCI-H460-Luc2 cells were maintained in RPMI 1640 medium (Hyclone, Logan, Utah, USA) mixed in the ratio of 1:1 with Opti-MEM (Invitrogen, Cat.22600-134) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium), counted and diluted to the concentration of 25×10⁶ cells/ml.

The HCT116 (in mice Crl:SHO-Prkdc^{scid}Hr^{hr}) were alternatively maintained in McCoy's medium (Hyclone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium), counted and diluted to the concentration of 25×10⁶ cells/ml. SW620 cells were maintained in DMEM (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium), counted and diluted to the concentration of 25×10⁶ cells/ml.

The HepG2cells were maintained in MEM (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4°C., 8 min., suspended in HBSS buffer (Hanks medium), counted and diluted to the concentration of 25×10⁶ cells/ml.

Mice

Examination of antitumor activity of proteins of the invention was conducted on 7-9 week-old CD-nude (Crl:CD1-Foxn1^{nu} 1) or 4-6 week-old Crl:SHO-Prkdc^{scid}Hr^{hr} mice obtained from Charles River Germany. Mice were kept under specific pathogen-free conditions with free access to food and demineralised water (ad libitum). All experiments on animals were carried in accordance with the guidelines: “Interdisciplinary Principles and Guidelines for the Use of Animals in Research. Marketing and Education” issued by the New York Academy of Sciences' Ad Hoc Committee on Animal Research and were approved by the IV Local Ethics Committee on Animal Experimentation in Warsaw (No. 71/2009).

The Course and Evaluation of the Experiments

Human Colon Cancer Model

Mice CD-Nude (Crl:CD1-Foxn1^{nu} 1)

HCT116 Model

On day 0 mice Crl:CD1-Foxn1^{nu} 1 were grafted subcutaneously (sc) in the right side with 5×10⁶ of HCT116 cells suspended in 0.2 ml HBSS buffer by means of a syringe with a 0.5×25 mm needle (Bogmark). When tumors reached the size of ˜55-68 mm³ (day 8), mice were randomized to obtain the average size of tumors in the group of ˜63 mm³ and assigned to treatment groups. The treatment groups were administered with the preparations of fusion protein of the invention of Ex. 2^{a} (10 mg/kg) and rhTRAIL114-281 (10 mg/kg) as a comparison. The preparations were administered intravenously (i.v.) following the scheme 10 daily applications with a two-day break after the first 5 applications. When a therapeutic group reached the average tumor size of −1000 mm³, mice were sacrificed by disruption of the spinal cord. The control group received rhTRAIL114-281.

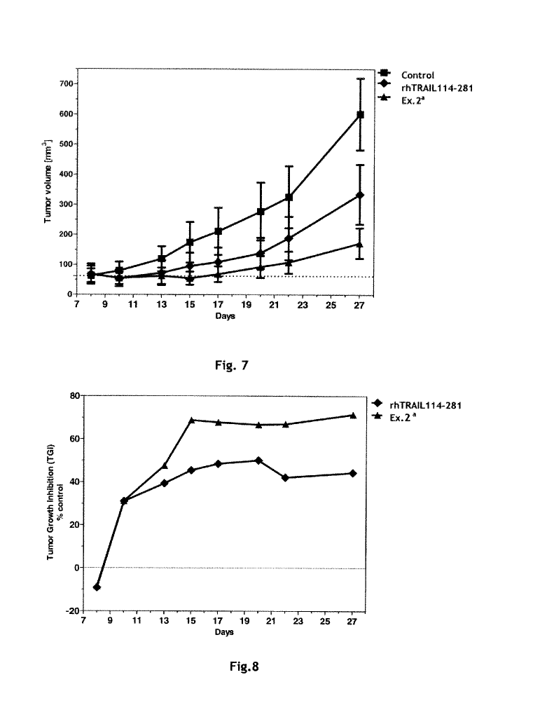

The experimental results obtained in mice Crl:CD1-Foxn1^{nu} burdened with HCT116 colon cancer treated with fusion proteins of the invention of Ex. 2^{a} and comparatively with rhTRAIL114-281 are shown in FIG. 7 as a diagram of changes of the tumor volume and in FIG. 8 which shows tumor growth inhibition (% TGI) as the percentage of control.

The experimental results obtained in mice Crl:CD1-Foxn1^{nu} burdened with HCT116 colon cancer treated with fusion protein of the invention of Ex. 2^{a} and comparatively with rhTRAIL114-281 are shown in FIG. 7 as a diagram of changes of the tumor volume and in FIG. 8 which shows tumor growth inhibition (% TGI) as the percentage of control.

The results of experiments presented in the graphs in FIGS. 7 and 8 show that administration of the fusion protein of the invention of Ex. 2^{a} caused tumor HCT116 growth inhibition, with TGI 71.2% relative to the control on 27^{th} day of the experiment. For rhTRAIL114-281 used as the comparative reference, a slight inhibitory effect on tumor cell growth was obtained relative to the control, with TGI at the level of 44%. Thus, fusion proteins of the invention exert much stronger effect compared to TRAIL alone.

On day 0 mice Crl:CD1-Foxn1^{nu} 1 were grafted subcutaneously (sc) in the right side with 5×10⁶ of HCT116 cells suspended in 0.2 ml HBSS buffer by means of a syringe with a 0.5×25 mm needle (Bogmark). When tumors reached the size of ˜50-110 mm³ (day 23), mice were randomized to obtain the average size of tumors in the group of ˜85 mm³ and assigned to treatment groups. The treatment groups were administered with the preparations of fusion protein of the invention of Ex. 8^{a} (10 mg/kg)and rhTRAIL114-281 (10 mg/kg) as a comparison. The preparations were administered intravenously (i.v.) daily for ten days. When a therapeutic group reached the average tumor size of ˜1000 mm³, mice were sacrificed by disruption of the spinal cord. The control group received rhTRAIL114-281.

The experimental results obtained in mice Crl:CD1-Foxn1^{nu} burdened with HCT116 colon cancer treated with fusion proteins of the invention of Ex. 8^{a} and comparatively with rhTRAIL114-281 are shown in FIG. 11 as a diagram of changes of the tumor volume and in FIG. 12 which shows tumor growth inhibition (% TGI) as the percentage of control.

The experimental results obtained in mice Crl:CD1-Foxn1^{nu} burdened with HCT116 colon cancer treated with fusion protein of the invention of Ex. 8^{a} and comparatively with rhTRAIL114-281 are shown in FIG. 11 as a diagram of changes of the tumor volume and in FIG. 12 which shows tumor growth inhibition (% TGI) as the percentage of control.

The results of experiments presented in the graphs in FIGS. 11 and 12 show that administration of the fusion protein of the invention of Ex. 8^{a} caused tumor HCT116 growth inhibition, with TGI 53.3 relative to the control on 31^{th} day of the experiment. For rhTRAIL114-281 used as the comparative reference, a slight inhibitory effect on tumor cell growth was obtained relative to the control, with TGI at the level of 21.8%. Thus, fusion proteins of the invention exert much stronger effect compared to TRAIL alone.

Mice Crl:SHO-Prkdc^{scid}Hr^{hr}

HCT116 Model

On day 0 mice Crl:SHO-Prkdc^{scid}Hr^{hr} were grafted subcutaneously (sc) in the right side with 5×10⁶ of HCT116 cells suspended in 0.1 ml 3:1 mixture of HBSS buffer:Matrigel by means of a syringe with a 0.5×25 mm needle (Bogmark). When tumors reached the size of 71-432 mm³ (day 13), mice were randomized to obtain the average size of tumors in the group of —180 mm³ and assigned to treatment groups. The treatment groups were administered with the preparations of fusion proteins of the invention of Ex. 8^{b} (50 mg/kg), and rhTRAIL114-281 (65 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0). The preparations were administered intravenously (i.v.) following the schema 10 daily applications with a two-day break after the first 5 applications.

When a therapeutic group reached the average tumor size of ˜1000 mm³, mice were sacrificed by disruption of the spinal cord. The control group received rhTRAIL114-281.

The experimental results obtained in mice Crl:SHO-Prkdc^{scid}Hr^{hr} burdened with HCT116 colon cancer treated with fusion protein of the invention of Ex.8, and comparatively with rhTRAIL114-281 are shown in FIG. 11a as a diagram of changes of the tumor volume, and in FIG. 12a which shows tumor growth inhibition (% TGI) as the percentage of control.

The results of experiments presented in the graphs in FIGS. 11a and 12a show that administration of the fusion protein of the invention Ex.8^{b} caused tumor HCT116 growth inhibition, with TGI 70% relative to the control on 24^{th} day of the experiment. For rhTRAIL114-281 used as the comparative reference, the slight inhibitory effect on tumor cell growth was obtained relative to the control, with TGI at the level of 38%. Thus, fusion protein of the invention exert much stronger effect compared to rhTRAIL114-281 alone.

SW620 Model

On day 0 mice Crl:SHO-Prkdc^{scid}Hr^{hr} were grafted subcutaneously (sc) in the right side with 5×10⁶ of SW620 cells suspended in 0.1 ml 3:1 mixture of HBSS buffer:Matrigel by means of a syringe with a 0.5×25 mm needle (Bogmark). When tumors reached the size of 280-340 mm³ (day 17), mice were randomized to obtain the average size of tumors in the group of ˜320 mm³ and assigned to treatment groups. The treatment groups were administered with the preparations of fusion proteins of the invention of Ex.8^{b} (40 mg/kg), and rhTRAIL114-281 (30 mg/kg) as a comparison against formulation buffer (5 mM NaH₂PO₄, 95 mM Na₂HPO₄, 200 mM NaCl, 5 mM glutathione, 0.1 mM ZnCl₂, 10% glycerol, 80 mM saccharose, pH 8.0). The preparations were administered intravenously (i.v.) six times every second day. When a therapeutic group reached the average tumor size of ˜1000 mm³, mice were sacrificed by disruption of the spinal cord. The control group received rhTRAIL114-281. The experimental results obtained in mice Crl:SHO-Prkdc^{scid}Hr^{hr} burdened with SW620 colon cancer treated with fusion protein of the invention of Ex. 8^{b}, and comparatively with rhTRAIL114-281 are shown in FIG. 13 as a diagram of changes of the tumor volume, and in FIG. 14 which shows tumor growth inhibition (% TGI) as the percentage of control.

The results of experiments presented in the graphs in FIGS. 13 and 14 show that administration of the fusion protein of the invention Ex. 8^{b} caused tumor SW620 growth inhibition, with TGI 44% relative to the control on 31^{st} day of the experiment. For rhTRAIL114-281 used as the comparative reference, the slight inhibitory effect on tumor cell growth was obtained relative to the control, with TGI at the level of −9%. Thus, fusion proteins of the invention exert much stronger effect compared to rhTRAIL114-281 alone.

Colo205 Model

On day 0 mice Crl:SHO-Prkdc^{scid}Hr^{hr} were grafted subcutaneously (sc) in the right side with 5×10⁶ of Colo205 cells suspended in 0.1 ml 3:1 mixture of HBSS buffer:Matrigel by means of a syringe with a 0.5×25 mm needle (Bogmark). When tumors reached the size of 108-128 mm³ (day 13), mice were randomized to obtain the average size of tumors in the group of ˜115 mm³ and assigned to treatment groups. The treatment groups were administered with the preparations of fusion proteins of the invention of Ex.8^{b} (30 mg/kg), and rhTRAIL114-281 (30 mg/kg) as a comparison against formulation buffer (5 mM NaH₂PO₄, 95 mM Na₂HPO₄, 200 mM NaCl, 5 mM glutathione, 0.1 mM ZnCl₂, 10% glycerol, 80 mM saccharose, pH 8.0). The preparations were administered intravenously (i.v.) six times every second day. When a therapeutic group reached the average tumor size of ˜1000 mm³, mice were sacrificed by disruption of the spinal cord. The control group received rhTRAIL114-281.

The experimental results obtained in mice Crl:SHO-Prkdc^{scid}Hr^{hr} burdened with Colo205 colon cancer treated with fusion protein of the invention of Ex. 8^{b}, and comparatively with rhTRAIL114-281 are shown in FIG. 15 as a diagram of changes of the tumor volume, and in FIG. 16 which shows tumor growth inhibition (% TGI) as the percentage of control.

The results of experiments presented in the graphs in FIGS. 15 and 16 show that administration of the fusion protein of the invention Ex. 8^{b} caused tumor Colo205 growth inhibition, with TGI 97.6% relative to the control on 33^{rd} day of the experiment. For rhTRAIL114-281 used as the comparative reference, the slight inhibitory effect on tumor cell growth was obtained relative to the control, with TGI at the level of 18.8%. Thus, fusion proteins of the invention exert much stronger effect compared to rhTRAIL114-281 alone.

Liver Cancer Model

Mice Crl:SHO-Prkdc^{scid}Hr^{hr}

HepG2 Model

On day 0 mice Crl:SHO-Prkdc^{scid}Hr^{hr} were grafted subcutaneously (sc) in the right side with 7×10⁶ of HepG2 cells suspended in 0.1 ml 3:1 mixture of HBSS buffer:Matrigel by means of a syringe with a 0.5×25 mm needle (Bogmark). When tumors reached the size of ˜313-374 mm³ (day 19), mice were randomized to obtain the average size of tumors in the group of ˜340 mm³ and assigned to treatment groups. The treatment groups were administered with the preparations of fusion protein of the invention of Ex. 8^{b} (30 mg/kg) and rhTRAIL114-281 (30 mg/kg) as a comparison against formulation buffer (5 mM NaH₂PO₄, 95 mM Na₂HPO₄, 200 mM NaCl, 5 mM glutatione, 0.1 mM ZnCl₂, 10% glycerol, 80 mM saccharose, pH 8.0) as a control. The preparations were administered intravenously (i.v.) six times every second day. When a therapeutic group reached the average tumor size of ˜1000 mm³, mice were sacrificed by disruption of the spinal cord. The control group received rhTRAIL114-281.

The experimental results obtained in mice Crl:SHO-Prkdc^{scid}Hr^{hr} burdened with HepG2 liver cancer treated with fusion protein of the invention of Ex. 8^{b} and comparatively with rhTRAIL114-281 are shown in FIG. 17 as a diagram of changes of the tumor volume, and in FIG. 18 which shows tumor growth inhibition (% TGI) as the percentage of control.

The results of experiments presented in the graphs in FIGS. 17 and 18 show that administration of the fusion proteins of the invention Ex. 8^{b} caused tumor HepG2 growth inhibition, with TGI 65.7% relative to the control on 33^{rd} day of the experiment. For rhTRAIL114-281 used as the comparative reference, the slight inhibitory effect on tumor cell growth was obtained relative to the control, with TGI at the level of 12.6%. Thus, fusion proteins of the invention exert much stronger effect compared to rhTRAIL114-281 alone.

Lung Cancer Model

Mice: Crl:CD1-Foxn1^{nu} 1

NCI-H460-Luc2 Model

On day 0 mice Crl:CD1-Foxn1nu 1 were grafted subcutaneously (sc) in the right side with 5×10⁶ of NCI-H460-Luc2 cells suspended in 0.1 ml HBSS buffer by means of a syringe with a0.5×25 mm needle (Bogmark). When tumors reached the size of ˜20-233 mm³ (day 16), mice were randomized to obtain the average size of tumors in the group of ˜110 mm³ and assigned to treatment groups. The treatment groups were administered with the preparations of fusion protein of the invention of Ex. 2^{a} (20 mg/kg) and rhTRAIL114-281 (10 mg/kg) as a comparison against formulation buffer f16 (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v.) six times every second day. When a therapeutic group reached the average tumor size of ˜1000 mm³, mice were sacrificed by disruption of the spinal cord. The control group received rhTRAIL114-281.

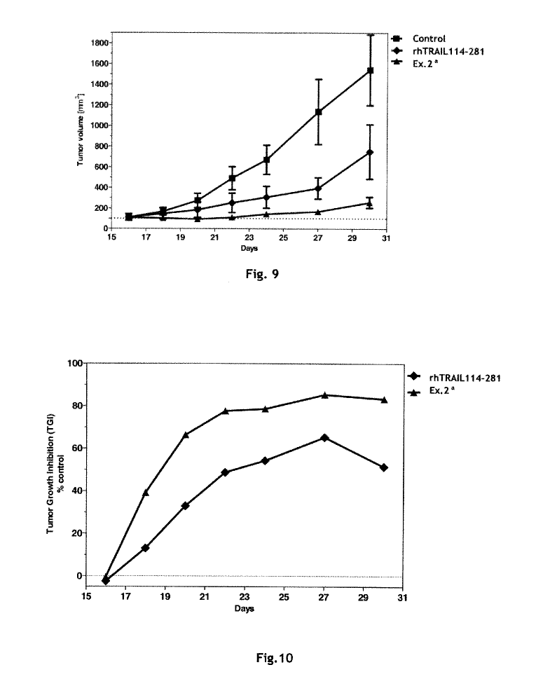

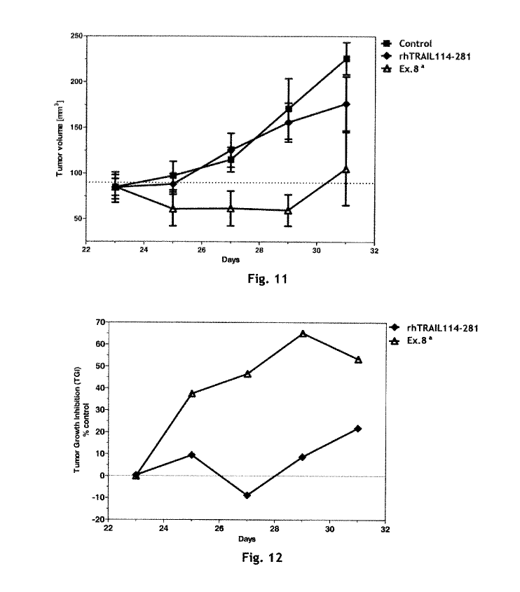

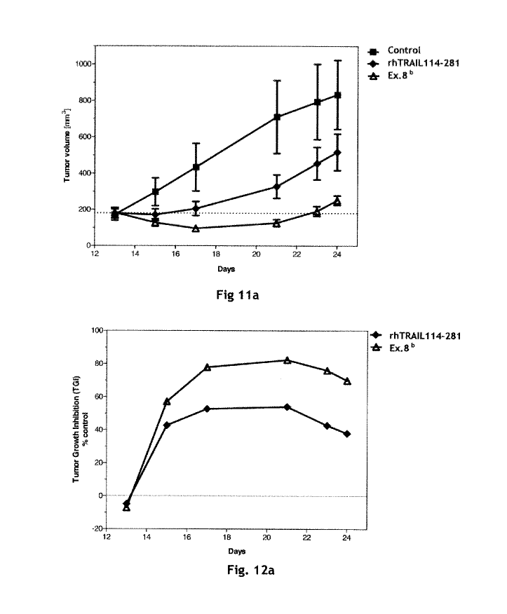

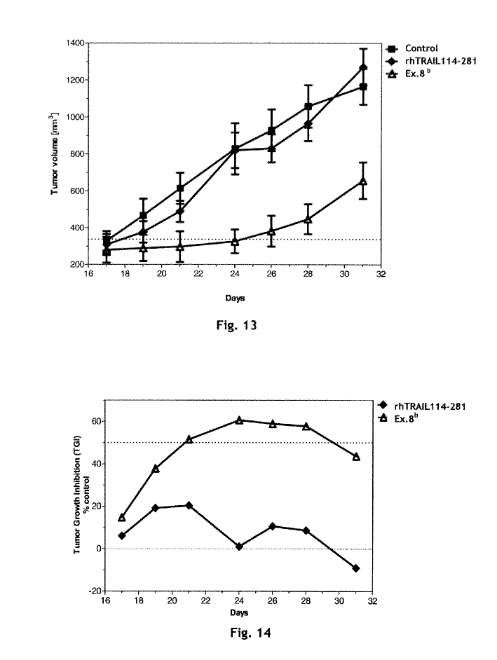

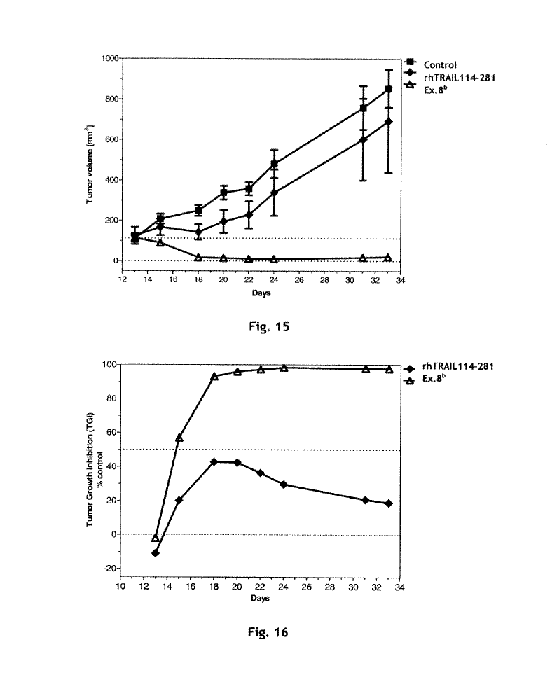

The experimental results obtained in mice Crl:SHO-Prkdc^{scid}Hr^{hr} burdened with NCI-H460-Luc2 lung cancer treated with fusion protein of the invention of Ex. 2^{a} and comparatively with rhTRAIL114-281 are shown in FIG. 9 as a diagram of changes of the tumor volume, and in FIG. 10 which shows tumor growth inhibition (% TGI) as the percentage of control.

The results of experiments presented in the graphs in FIGS. 9 and 10 show that administration of the fusion protein of the invention Ex. 2^{a} caused tumor NCI-H460-Luc2 growth inhibition, with TGI 81.3% relative to the control on 30^{th} day of the experiment. For rhTRAIL114-281 used as the comparative reference, a slight inhibitory effect on tumor cell growth was obtained relative to the control, with TGI at the level of 53.1%. Thus, fusion proteins of the invention exert much stronger effect compared to rhTRAIL114-281 alone.

Mice: Crl:SHO-PrkdcscidHrhr

NCI-H460 Model

On day 0 mice Crl:SHO-PrkdcscidHrhr were grafted subcutaneously (sc) in the right side with 5×10⁶ of NCI-H460 cells suspended in 0.1 ml HBSS buffer by means of a syringe with a 0.5×25 mm needle (Bogmark). When tumors reached the size of ˜150-178 mm³ (day 13), mice were randomized to obtain the average size of tumors in the group of ˜160 mm3 and assigned to treatment groups. The treatment groups were administered with the preparations of fusion protein of the invention of Ex. 8^{b} TRP5 (30 mg/kg) and rhTRAIL114-281 (30 mg/kg) as a comparison against formulation buffer (5 mM NaH₂PO₄, 95 mM Na₂HPO₄, 200 mM NaCl, 5 mM glutathione, 0.1 mM ZnCl₂, 10% glycerol, 80 mM saccharose, pH 8.0) as a control. The preparations were administered intravenously (i.v.) six times every second day. When a therapeutic group reached the average tumor size of ˜1000 mm³, mice were sacrificed by disruption of the spinal cord. The control group received rhTRAIL114-281.

The experimental results obtained in mice Crl:SHO-Prkdc^{scid}Hr^{hr} burdened with NCI-H460 lung cancer treated with fusion protein of the invention of Ex.8^{b} and comparatively with rhTRAIL114-281 are shown in FIG. 19 as a diagram of changes of the tumor volume, and in FIG. 20 which shows tumor growth inhibition (% TGI) as the percentage of control.

The results of experiments presented in the graphs in FIGS. 19 and 20 show that administration of the fusion protein of the invention Ex. 8^{b} caused tumor NCI-H460 growth inhibition, with TGI 61% relative to the control on 28^{th} day of the experiment. For rhTRAIL114-281 used as the comparative reference, a slight inhibitory effect on tumor cell growth was obtained relative to the control, with TGI at the level of 17.5%. Thus, fusion proteins of the invention exert much stronger effect compared to rhTRAIL114-281 alone.

The tested fusion proteins did not cause significant side effects manifested by a decrease in body weight of mice (i.e. less than 10% of the baseline body weight). This shows low systemic toxicity of the protein.

1. A fusion protein comprising:
domain (a) which comprises the functional fragment of a soluble hTRAIL protein sequence starting with an amino acid in a position not lower than hTRAIL95, or a homolog of said functional fragment having at least 70% sequence identity; and
at least one domain (b) which is the sequence of an effector peptide having anti-proliferative activity against tumour cells,
wherein the sequence of domain (b) is attached at the C-terminus and/or at the N-terminus of domain (a).

2. The fusion protein according to claim 1, wherein domain (a) comprises a fragment of soluble hTRAIL (SEQ. No. 78) protein sequence starting with an amino acid in the range from hTRAIL95 to hTRAIL121, inclusive, and ending with the amino acid 281.

3. The fusion protein according to claim 1, wherein domain (a) is selected from the group consisting of hTRAIL95-281, hTRAIL114-281, hTRAIL119-281, hTRAIL120-281, and hTRAIL121-281.

4. The fusion protein according to claim 1, wherein domain (b) is selected from the group consisting of:
16-amino acid peptide blocking FGF-2 receptor of SEQ. No. 26;
34 amino acid fragment of human fetoprotein of SEQ. No. 27;
8-amino acid fragment of human fetoprotein of SEQ. No. 28;
peptide derived from p21^{WAF} of SEQ. No. 29;
peptide DD2 from DOC-2/DAB2 protein of SEQ. No. 30;
arginine deiminase from Mycoplasma arginini of SEQ. No. 31;
fragment of p16 peptide of SEQ. No. 32;
fragment of p16 peptide fused with a 17-amino-acid transporting domain of antennapedia of SEQ. No. 33;
fragment of MEK-1 protein of SEQ. No. 34;
N terminal fragment of PH domain of TCL1 protein of SEQ. No. 35;
hexapeptide Phe-Trp-Leu-Arg-Phe-Thr of SEQ. No. 36;
13-amino acid tubulin fragment of SEQ. No. 37;
10-amino acid tubulin fragment of SEQ. No. 38;
melittin of SEQ. No. 39;
6-amino acid peptide C2 derived from bee defensin of SEQ. No. 40;
8-amino acid peptide binding to FGF-2 ligand of SEQ. No. 41;
15-amino acid lasioglossin LL2 peptide of SEQ. No. 42;
13-amino acid peptide binding to SH3 RasGAP domain of SEQ. No. 43; and
analogue of Pep27 peptide of SEQ. No. 44.

5. The fusion protein according to claim 1, which between domain (a) and domain (b) contains domain (c) comprising a protease cleavage site, selected from a sequence recognized by metalloprotease MMP, a sequence recognized by urokinase uPA, and combinations thereof.

6. The fusion protein according to claim 5, wherein the sequence recognized by metalloprotease MMP is SEQ. No. 45, and the sequence recognized by urokinase uPA is SEQ. No. 46.

7. The fusion protein according to claims 5, wherein domain (c) is a combination of sequences recognized by metalloprotease MMP and urokinase uPA located next to each other.

8. The fusion protein according to claim 1, wherein domain (b) is additionally linked with a transporting domain (d) selected from the group consisting of:
(d1) a fragment of antennapedia protein domain of SEQ. No. 48,
(d2) a fragment of antennapedia protein domain of SEQ. No. 49,
(d3) polyarginine sequence transporting through a cell membrane, consisting of 6, 7, 8, 9, 10 or 11 Arg residues, and
combinations thereof.

9. The fusion protein according to claim 8, wherein the sequence (d) is located at the C-terminus or at the N-terminus of the fusion protein.

10. The fusion protein according to claim 8, wherein the transporting sequence (d) is located between domains (b) and (c).

11. The fusion protein according to claim 8, wherein the sequence (d) is located at the C-terminus of the fusion protein.

12. The fusion protein according to claim 1, which additionally comprises a flexible steric linker between domains (a), (b), (c) and/or (d).

13. (canceled)

14. The fusion protein according to claim 1, having the amino acid sequence selected from the group consisting of SEQ. No. 1; SEQ. No. 2; SEQ. No. 3; SEQ. No. 4; SEQ. No. 5; SEQ. No. 6; SEQ. No. 7; SEQ. No. 8; SEQ. No. 9; SEQ. No. 10; SEQ. No. 11; SEQ. No. 12; SEQ. No. 13; SEQ. No. 14, SEQ. No. 15, SEQ. No. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25, and SEQ. No. 75.

15-25. (canceled)

26. A pharmaceutical composition, comprising as an active ingredient the fusion protein as defined in claim 1, in combination with a pharmaceutically acceptable carrier.

27. A pharmaceutical composition, comprising as an active ingredient the fusion protein as defined in claim 8, in combination with a pharmaceutically acceptable carrier.

28. A pharmaceutical composition, comprising as an active ingredient the fusion protein as defined in claim 14, in combination with a pharmaceutically acceptable carrier.

29. A method of treating cancer in a mammal, in a need thereof, which comprises administering to the mammal an anti-neoplastic-effective amount of the fusion protein as defined in claim 1.

30. A method of treating cancer in a mammal, in a need thereof, which comprises administering to the mammal an anti-neoplastic-effective amount of the fusion protein as defined in claim 14.
